(19) United States
(12) Patent Application Publication
(10) Pub. No.: US 2024/0209447 A1
(43) Pub. Date: Jun. 27, 2024

(54) COMPRESSIVE MOLECULAR PROBES FOR GENOMIC EDITING AND TRACKING

(71) Applicant: The Board of Trustees of the Leland Stanford Junior University, Stanford, CA (US)

(72) Inventors: Le Cong, Stanford, CA (US); Nicholas W. Hughes, Stanford, CA (US)

(21) Appl. No.: 18/287,619

(22) PCT Filed: Apr. 20, 2022
(86) PCT No.: PCT/US2022/025582

§ 371 Date: Oct. 19, 2023

Related U.S. Application Data
(60) Provisional application No. 63/177,187, filed on Apr. 20, 2021.

(51) Int. Cl. C12Q 1/6886; C12N 9/22; C12N 15/11; C12Q 1/44; C12Q 1/6813; G16B 20/50; G16B 25/30

(52) U.S. Cl. CPC C12Q 1/6886; C12N 9/22; C12N 15/111; C12Q 1/44; C12Q 1/6813; G16B 20/50; G16B 25/30; C12N 2310/20; C12Q 2600/158; G01N 2333/922

(57) ABSTRACT

The present disclosure provides systems, methods, nucleic acids, and kits for barcoding and tracking cells based on CRISPR editing technologies.

### CROSS-REFERENCE TO RELATED APPLICATIONS

This application claims the benefit of U.S. Provisional Application No. 63/177,187, filed Apr. 20, 2021, the contents of which is herein incorporated by reference in its entirety.

### STATEMENT REGARDING FEDERALLY SPONSORED RESEARCH OR DEVELOPMENT

This invention was made with government support under contract 1953415 awarded by the National Science Foundation and under contract HG011316 awarded by the National Institutes of Health. The government has certain rights in the invention.

### FIELD

The present invention relates to systems, methods, nucleic acids, and kits for barcoding and tracking cells.

### SEQUENCE LISTING

The text of the computer readable sequence listing filed herewith, titled “39428-601_SEQUENCE_LISTING_ST25”, created Apr. 20, 2022, having a file size of 204,272 bytes, is hereby incorporated by reference in its entirety.

### BACKGROUND

In a biological systems and in most human diseases, millions and often times billions of cells are involved a complex patho-physiological process, such as cancer or neurological disorders. For example, a standard breast cancer biopsy will contain over 200 million cells, and some of the rare cells within the biopsy may be the true diagnostic- or therapeutic-relevant cell types for the patient. Similarly, in a typical study of brain disorders, hundreds of millions of neurons are present in the part of the brain that may be causal to the pathology of disease such as Alzheimer's or Parkinson's diseases, all of them should be genetically perturbed or measured systematically to identify the key disease-causing cells or genetic changes.

Nonetheless, typical experiments or analysis in laboratory or industrial settings only sample a relatively small number of cells, from thousands to at most millions of cells. This is insufficient to probe the complex biology and pathology described above. The process of engineering, editing, or measuring cell needs to be affordable at massive scale. In recent years, gene-editing approaches such as CRISPR-Cas9 genome-wide screening have developed systematically with gene expression of many cells being measured using Next-Generation-Sequencing (NGS). While these methods are efficient and useful, they are extremely labor- and cost-intensive. Hence, a majority of scientific research in labs is done at the scale that severely limits the understanding of cellular and disease biology, let alone to systematically modify and edit the molecules within a cell to understand their function.

### SUMMARY

Provided herein are systems, components, and methods for barcoding and tracking cells. In some embodiments the system comprises: a Cas12a protein or a vector encoding thereof; a polynucleotide barcode flanked by two PAM sequences of inverse orientation, or a vector encoding thereof, wherein the polynucleotide barcode comprises a first target nucleic acid sequence and a second target nucleic acid sequence; and a pair of guide RNAs (gRNAs) configured to hybridize to the two PAM sequences. In some embodiments, the system comprises: a CRISPR associated (Cas) endonuclease or a vector encoding thereof; a polynucleotide barcode comprising less than 100 nucleotides flanked by two PAM sequences: and a pair of guide RNAs (gRNAs) configured to hybridize to the two PAM sequences. In some embodiments, the two PAM sequences are of inverse orientation. In some embodiments, the Cas endonuclease is a Class 2 Cas endonuclease. In some embodiments, the Cas endonuclease is a Type V Cas endonuclease. In some embodiments, the Cas endonuclease is selected from Cas9, Cas12a, and Cas14.

In some embodiments, the polynucleotide barcode is on the vector encoding the pair of RNAs. In some embodiments, the polynucleotide barcode is on the vector encoding the pair of gRNAs. In some embodiments, the vector encoding the Cas12a protein is the same vector as the vector encoding the pair of gRNAs. In some embodiments, the polynucleotide barcode is on same vector as the pair of gRNAs and the Cas12a protein.

In some embodiments, polynucleotide barcode further comprises a linker between the first target nucleic acid sequence and the second target nucleic acid sequence. In some embodiments, the linker comprises 1-20 nucleotides. In some embodiments, the linker comprises 10 nucleotides.

In some embodiments, the polynucleotide barcode comprises, comprises less than 200 nucleotides, less than 150 nucleotides or less than 100 nucleotides. In some embodiments, the polynucleotide barcode comprises 50-60 nucleotides. In select embodiments, the polynucleotide barcode comprises 54 nucleotides.

In some embodiments, the polynucleotide barcode sequence is configured to promote insertions and deletions over time. In some embodiments, the polynucleotide barcode comprises GC directly upstream of PAM sequence at the 3′ end of the polynucleotide barcode. In some embodiments, the polynucleotide barcode comprises a cytidine at position 39 and a guanosine at position 40. In some embodiments, the polynucleotide barcode comprises an adenosine at positions 45 and 46. In some embodiments, the polynucleotide barcode comprises an adenosine at position 31 and a guanosine at position 32. In some embodiments, the polynucleotide barcode does not comprise a thymidine at position 54, position 49, or both. In some embodiments, the polynucleotide barcode does not comprise a guanosine at position 50 and a cytidine at position 51. In some embodiments, the polynucleotide barcode comprises CACTTG (SEQ ID NO: 1054) at positions 32-37. In some embodiments, the polynucleotide barcode comprises CCTAGTAATAG (SEQ ID NO: 1055) at positions 39-49. In some embodiments, the polynucleotide barcode comprises CCGG (SEQ ID NO: 1056) directly downstream of PAM sequence at the 5′ end of the polynucleotide barcode. In some embodiments, the polynucleotide barcode comprises a sequence with at least 70% similarity (e.g., at least 80%, at least 90%, at least 95%, at least 95%) to sequences selected from the group consisting of SEQ ID NOs: 1-1053. In some embodiments, the polynucleotide barcode sequence is selected from the group consisting of SEQ ID NOs: 1-10. In some embodiments, the polynucleotide barcode comprises a sequence of any of SEQ ID Nos: 1-1053 wherein in one or more (e.g., one, two, three, four, five, six, seven, eight, nine, ten) nucleotides is substituted with a different natural or synthetic nucleotide.

In some embodiments, the pair of gRNAs are within a crRNA array. In some embodiments, one or each of the gRNAs comprise a guide sequence of less than 25 nucleotides.

In some embodiments, the system further comprises at least one gRNA configured to hybridize to a recipient nucleic acid. In some embodiments, the recipient nucleic acid is a nucleic acid endogenous to a target cell. In some embodiments, the recipient nucleic acid is a gene within a target cell. In some embodiments, the at least one gRNA configured to hybridize to a recipient nucleic acid is on the vector encoding the two gRNAs. In some embodiments, the at least one gRNA is within the crRNA array comprising the two gRNAs. In some embodiments, at least one or all of the gRNAs are non-naturally occurring gRNAs. In some embodiments, the system further comprises a recipient nucleic acid.

In some embodiments, the vector encoding Cas12a or the Cas endonuclease comprises an inducible promoter for Cas12a or Cas endonuclease expression.

In some embodiments, the systems further comprise a gene editing system. In some embodiments, the gene editing system comprises a CRISPR/Cas gene editing system. In some embodiments, the system further comprises one or more gene editing gRNAs. In some embodiments, the one or more gene editing gRNA are provided in a crRNA array with the pair of guide RNAs.

Also provided are cells or a population of cells comprising the present systems. In some embodiments, the populations of cells comprise a distinct version of the barcode representing a particular cell generation or cell lineage (e.g., each distinct versions comprises distinct insertions or deletions within the barcode sequence (FIG. 1B)). In some embodiments, the population of cells represents up to 1000 cell generations or cell lineages (e.g., about 100, about 200, about 300, about 400, about 500, about 600, about 700, about 800 or about 900) cell generations. In some embodiments, the population of cells represents about 700 cell generations.

The disclosure also provides a nucleic acid comprising the polynucleotide barcode as described herein. In some embodiments, the nucleic acid encodes a pair of gRNAs configured to hybridize to the two PAM sequences flanking the polynucleotide barcode. In some embodiments, the pair of gRNAs are within a crRNA array. In some embodiments, the crRNA array further comprises a termination signal for crRNA expression. In some embodiments, the crRNA array further comprises at least one gRNA configured to hybridize to a recipient nucleic acid. In some embodiments, the nucleic acid further comprises a sequence tag configured to remain static over time. In some embodiments, the nucleic acid further comprises one or more gene editing gRNAs.

Further provided are methods for introducing a polynucleotide barcode in a cell comprising introducing into the cell the present systems or the present nucleic acid and a CRISPR associated (Cas) endonuclease or a nucleic acid encoding a CRISPR associated (Cas) endonuclease. In some embodiments, the Cas endonuclease is a Class 2 Cas endonuclease. In some embodiments, the Cas endonuclease is a Type V Cas endonuclease. In some embodiments, the Cas endonuclease is selected from Cas9, Cas12a, and Cas14. In some embodiments, the polynucleotide barcode integrates into genomic DNA. In some embodiments, the polynucleotide barcode is passed to daughter cells. In some embodiments, the cell is eukaryotic cell. In some embodiments, the cell is in vitro, ex vivo, or in a subject (e.g., in vivo).

Additionally, the disclosure provides methods for cell tracking comprising introducing into the cell the present systems or the present nucleic acid and a CRISPR associated (Cas) endonuclease or a nucleic acid encoding a CRISPR associated (Cas) endonuclease; isolating cellular nucleic acids at one or more time points; sequencing the polynucleotide barcode at the one or more time points; and tracking changes to original sequence of barcode in the cell at each time point. In some embodiments, the cell is eukaryotic cell. In some embodiments, the cell is in vitro, ex vivo, or in a subject (e.g., in vivo).

In some embodiments, the polynucleotide barcode integrates into genomic DNA. In some embodiments, the polynucleotide barcode is passed to daughter cells. In some embodiments, the one or more time points are over multiple cell generations. In some embodiments, the methods further comprise establishing lineage connections or a sequence of changes in barcode sequence between cells from different generations.

In some embodiments, expression of Cas12a or the Cas endonuclease is controlled by an inducible promoter. In some embodiments, the methods further comprise adding varying concentrations of an inducing agent to the cells to vary the change rate of the original barcode sequence. In some embodiments, increasing concentrations of the inducing agent increases the change rate of the original barcode sequence.

In some embodiments, the methods further comprise determining single-cell transcriptomic profiles. In some embodiments, the methods further comprise characterizing heritability of gene expression patterns and/or determining gene products which have heritable expression patterns.

In some embodiments, the methods further comprise introducing one or more mutations, insertions, or deletions in one or more target genes of interest in the cell. In some embodiments, the methods further comprise monitoring the effect of the one or more mutations, insertions, or deletions on cell function, cell viability, or effectiveness of a pharmacological treatment.

Also disclosed is a computer implemented method for designing a polynucleotide barcode sequence configured to promote insertions and deletions over time comprising: designing a seed barcode sequence based on sequence elements which promote insertions and deletions, sequence elements which suppress insertions and deletions, or both; iteratively mutating the seed barcode sequence; and predicting sequence entropy as measure of insertions and deletions accumulated in the polynucleotide barcode sequence over time.

In some embodiments, the sequence elements which promote insertions and deletions are selected from the group consisting of: a GC dinucleotide at the 3′ end of the barcode; a CG dinucleotide starting at position 39; an AA dinucleotide starting at position 45; an AG dinucleotide starting at position 31; CACTTG (SEQ ID NO: 1054) at positions 32-37; CCTAGTAATAG (SEQ ID NO: 1055) at positions 39-49; CCGG (SEQ ID NO: 1056) at the 5′ end of the polynucleotide barcode; or a combination thereof.

In some embodiments, the sequence elements which suppress insertions and deletions are selected from the group consisting of: a thymidine at position 54; a thymidine at position 49; a GC dinucleotide starting at position 50; or a combination thereof.

Other aspects and embodiments of the disclosure will be apparent in light of the following detailed description and accompanying figures.

### BRIEF DESCRIPTION OF THE DRAWINGS

FIGS. 1A-1B are schematic representations of an exemplary evolvable barcode. FIG. 1A is an illustration of an exemplary design of a barcode system, in which a single crRNA array with two guides (G1/G2) could be processed to edit two target sites within a barcode. Continuous editing generates evolvable barcodes. FIG. 1B shows editing outcomes (SEQ ID NOs: 1101 and 1102) at the target sites (T1 (SEQ ID NO: 1099)/T2 (SEQ ID NO: 1100)) within a barcode are used to place cells within a lineage tree, which together with single-cell RNA-Seq can recover simultaneous transcriptomic profiles.

FIGS. 2A-2I show the comparison of Cas9 and Cas12a for gene editing-based cell barcoding. FIG. 2A is a schematic of an exemplary design of synthetic barcode experiments to compare Cas12a/Cas9 using lentiviral vectors and doxycycline-inducible cell lines. FIG. 2B is a schematic of exemplary vector designs for Cas12a editing (top) and Cas9 editing (middle) of a common two-target barcode (bottom). Three published barcodes were picked from a published Cas9 study. FIG. 2C are graphs of entropy of editing outcomes after doxycycline-induced Cas12a/Cas9 expression. FIG. 2D is a stacked bar chart comparing the editing outcome distribution of Cas12a/Cas9 barcodes. Bar areas correspond to the sequencing reads frequency of each unique indel outcome, with total counts listed on the right. FIG. 2E is a schematic of an exemplary design of endogenous editing experiments to compare Cas12a/Cas9 using transient transfection. FIG. 2F is a graph of gene-editing efficiencies across endogenous targets showing comparable levels of indel formation between Cas12a/Cas9. FIG. 2G is endogenous target sequences indicating the proximal PAM sequences (Cas12a in blue, Cas9 in purple) for DNMT1 (SEQ ID NO: 1103), CCR5 (SEQ ID NO: 1104) and AAVS1 (SEQ ID NO: 1105). FIG. 2H is a graph of entropy of Cas12a and Cas9-based editing outcomes at endogenous targets. FIG. 2I is a stacked bar chart comparing editing outcome distribution as in FIG. 2D. Unless otherwise noted, all statistical comparison in this and following figures were performed via a t-test with 1% false-discovery rate (FDR) using a two-stage step-up method of Benjamini, Krieger and Yekutieli, * (p<0.05); ** (p<0.01); *** (p<0.001).

FIGS. 3A and 3H show the design and high-throughput screening of DAISY barcodes. FIG. 3A is a schematic of an exemplary dual acting inverted site array (DAISY) barcode design with two crRNA-target pairs. The guide sequences were selected to have phased efficiency (DeepCpf1) and low off-target scores (FlashFry), see Examples for details. FIG. 3B is a schematic of the process for DAISY barcode sequences are synthesized and packaged into lentivirus for cell delivery. Pooled screening with next-generation sequencing is used to evaluate the entropy of each barcode sequence across multiple timepoints. FIG. 3C is a graph of the distribution of barcode entropies across all DAISY barcodes at each timepoint. FIG. 3D is a scatterplot of barcode entropy measured at Day 14 from two biological replicates, showing consistent results from separate lentiviral transductions. FIG. 3E is a graph of the correlation between measured barcode entropy and predicted editing efficiency scores (average of two target sites) using DeepCpf1. FIG. 3F is a graph of indel length distribution across all barcodes where the minimum inter-site deletion length is indicated. FIG. 3G is the Pearson Correlation Coefficients (PCC) between indel outcome types at each timepoint and the final barcode entropy across all DAISY barcodes. FIG. 3H is a graph of the comparison of DAISY barcode entropy to screen references and meta-analysis of published Cas9 barcoding data.

FIGS. 4A-4G show machine learning optimization of DAISY barcodes using CLOVER pipeline to predict and generate optimized DAISY barcodes with high-capacity and tunability. FIG. 4A is a schematic of the overall design of CLOVER pipeline to optimize DAISY barcode sequences via iterative pooled screening and machine learning modeling. FIG. 4B is a graph showing the linear ridge regression model accurately predicts entropy of DAISY barcodes. FIG. 4C is a graph of the distributions of barcode entropy from DAISY barcodes in 1st screen (initial pool) and from 2nd screen (CLOVER-optimized) in A375 cells. FIG. 4D is a graph of the comparison of barcode entropy demonstrating consistent performance of CLOVER-optimized DAISY barcodes in melanoma and lung adenocarcinoma cell lines. Top barcodes used in later experiments are highlighted. FIG. 4E is a schematic of an exemplary experiment design to measure doxycycline-dependent tunability of top DAISY barcodes in A375 cells. Low and High-dox were 40 and 1000 ng/mL. FIG. 4F is a graph of the change in the barcode entropy over time using low and high-dox. FIG. 4G is the rate kinetics of barcode entropy (based on the Exponential plateau model) across doxycycline dosages and biological replicates.

FIGS. 5A-5M show single-cell demonstration with optimized DAISY barcodes recovers lineage history and transcriptomic information. FIG. 5A is a schematic of an exemplary design of single-cell experiment using lentiviral delivery of an optimized DAISY barcode (scDAISY-seq). FIG. 5B is a graph of the distribution of editing outcomes within the DAISY barcode (BC) region. Barcode entropy from single-cell data shown on right. FIG. 5C shows unique barcode sequences recovered from scRNA-seq with yellow marks deletions and dark blue marks insertions. FIG. 5D is a lineage tree reconstructed from single-cell barcode sequences of largest Clone 1 (C1), read counts shown in log scale. Pie charts on the right showing the cell distribution of identified unique lineages. FIG. 5E is a homoplasy check showing no overlap between DAISY barcode sequences recovered from the largest two clones C1 and C2. FIG. 5F is a plot of barcoding capacity (rounded to thousands, log scale) versus barcode size (log scale) for CRISPR barcodes with demonstrated single-cell RNA-seq readouts. FIG. 5G is a reconstructed lineage tree from C1 using DAISY barcodes. Observed edits are illustrated below leaves of the tree. Purple and green bars indicate edits within two target sites. Heatmaps indicate cell numbers after quality filtering. FIG. 5H is an illustration of transcriptional memory showing that an expressed gene (amber) can exhibit non-heritable/heritable expression patterns depending on if its expression level persists within certain lineages. FIG. 5I, left, is a quantitative definition of a memory index using single-cell transcriptomic data with randomized (x-axis) vs. barcode-defined (y-axis) lineage assignments. FIG. 5I, right, is data from scDAISY-seq analyzed to calculate memory index for each gene. CV is the coefficient of variation of gene expression (see Methods). FIG. 5J is the distribution of memory index values across all genes. FIG. 5K shows the top significantly enriched gene sets from found high memory genes. FIG. 5L shows the top 5 proteins enriched proximally to the high memory genes (90 percentile) based on ENCODE data. FIG. 5M is ChIP-Seq peak profiles of high memory genes (90 percentile) in blue versus control genes (expression-matched, see Methods) in grey.

FIGS. 6A and 6B show a comparison between Cas9 and Cas12a barcode systems and features. FIG. 6A are schematics of exemplary Cas9 barcode systems contain multiplexed sgRNAs from separate U6 promoters, which can be contained in a single vector with target sites (top) or two vectors (middle). In contrast, a single vector with one U6 promoter is needed for Cas12a barcoding (bottom). FIG. 6B is a table of key parameters that characterize the complexity of crRNA cloning and potential complications and considerations, like off-target effects, when performing multiplexed genome editing with either Cas9 or Cas12a.

FIGS. 7A and 7B show the establishment of doxycycline-inducible Cas12a/Cas9 cell lines. FIG. 7A shows a vector schematic showing mKate2 fusion to either Cas12a or Cas9 under a TRE and a separate EF1-alpha-driven ORF expressing rtTA3-p2a-puroR (top) and micrographs showing doxycycline-dependent expression of mKate2 (bottom). FIG. 6B are graphs of the relationship between FACS-based measurement of mKate2 intensity (top), which is linked to Cas12a through a t2a peptide, and doxycycline dose, and the log-transform of the data (bottom).

FIG. 8 is a graph of doxycycline-dependent genome editing with a crRNA designed against VEGFA versus a non-targeting control over time.

FIG. 9 is a heatmap indicating the frequency (number of reads) of indel events with different lengths across endogenous loci that contain proximal Cas12a and Cas9 PAMs.

FIGS. 10A-10C are graphs of the quantification of edits at the second target site in the presence or absence of an inter-site deletion (PAM collapsing) event across three barcodes tested.

FIG. 11 is a schematic showing the design principles of the DAISY barcode in which indels are targeted centrally within the dual-target region with phased efficiencies.

FIGS. 12A-12C show computational assembly of the DAISY barcode screen library. FIG. 12A is a schematic of DAISY library design workflow. The final DAISY cassette is shown in which two crRNAs and two target sites are assembled, generating the final cassettes which are uniquely identifiable by the design-BC for screening. FIG. 12B is a graph showing the 5000 randomly generated sequences were filtered based on a custom off-target score that takes into account the frequency of off-target sites and the Hamming distance between the off-target site and the guide sequence. FIG. 12C is a construction of DAISYs using DeepCpf1 efficiency scoring and pairwise assembly into phased designs with filtering out of polyT sequences that may inhibit PolIII transcription.

FIG. 13 is an exemplary schematic showing feature space construction within the CLOVER pipeline, including both nucleotide-based features (top—SEQ ID NO: 1106) and a distance-based encoding of MMEJ features.

FIG. 14 is an exemplary vector design for single-cell barcoding with DAISY barcodes where the DAISY barcode includes an additional guide for potential genomic editing along with a static randomized tag that is captured with the 10× capture sequence.

FIGS. 15A-15C show cell cycle-related gene expression states contribute to clustering. FIG. 15A is a UMAP embedding of gene expression states of barcoded cells. FIG. 15B is scoring of each cell by expression level of genes associated with S-phase shows enrichment of high S-scores in cluster 2. FIG. 15C is scoring by expression level of genes associated with G2M-phase shows enrichment of high G2M-scores in cluster 3.

FIGS. 16A and 16B show EZH2 expression levels correlate with total heterogeneity of gene expression within SKCM lesions from the TCGA. FIG. 16A is a graph of the relationship between the total EZH2 expression level within tumors and the heterogeneity of gene expression across all genes (transcriptional heterogeneity) quantified as the Shannon diversity index. Tumors are binned into four groups based upon the maximum observed EZH2 expression level. Cells expressing less than a quarter, half, three-quarters, and unity of the expression maxi-mum are in the first, second, third, and fourth bins, respectively. FIG. 16B is a distribution of spearman rank correlation values of the expression level of a gene and the transcriptional heterogeneity within a tumor across all tumors. EZH2 is plotted with GAPDH as a reference.

FIGS. 17A-17D are plots showing sequence motif analysis for DAISY barcodes as determined by CLOVER having the top ten (FIG. 17A), the top fifty (FIG. 17B) and the top one hundred (FIG. 17C) barcodes as ranked by sequence contributions related to high entropy and a comparison of the top ten and bottom ten barcode sequences (FIG. 17D).

FIGS. 18A-18C show the ability to perform multiplexed editing of a gene of interest along with DAISY barcode editing (DAISY×gene editing, referred to as DAISY×G). FIG. 18A is a graph of competitive outgrowth in which a DAISY×G vector was used to introduce drug-resistance conferring mutations to BRAFi (Dabrafenib) treatment in melanoma cells. NTC refers to a control not targeting the genome; cr1 and cr2 refer to designs targeting one of a single locus within NF2: and dbl refers to designs targeting both loci within the same cell. FIG. 18B is a graph confirming increased frequency of indel mutation formation within the NF2 loci after drug treatment with a BRAF inhibitor (Dabrafenib). FIG. 18C shows the use of DAISY barcode sequencing to provide sub-clonal resolution of drug resistant colonies. The representation of sub-clones (marked by unique DAISY alleles) is well-correlated across biological replicates deriving from the same parental cell population, suggesting that there is a pre-existing state within NF2-null cell populations that is outcompeting other clonal populations.

FIG. 19 shows the use of DAISY barcoding for determining transcriptional memory effects and its relationship to potential drug discoveries. Inhibition of EZH2, an epigenetic regulator of transcriptional memory as proposed by DAISY barcoding, is synergistic with BRAF inhibition using small molecules.

### DETAILED DESCRIPTION OF THE INVENTION

Recent advancement of CRISPR Cas9-based genetic barcoding presents an exciting opportunity to understand the developmental trajectories of individual cells. To track a large number of lineages within tunable time frames, disclosed herein is a barcoding system, named Dual Acting Inverted Site arraY (DAISY) barcode, which is exemplified using Cas12a and is compact, tunable and high-capacity. By combining high-throughput screening and machine-learning optimization, an iterative experimental-computational pipeline for predicting and optimizing DAISY barcode sequences was developed. This pipeline generated a collection of high-capacity Cas12a barcode sequences. The top-performing barcodes predicted by the model achieved up to 5-bits improvement in tracking capacity. These optimized DAISY barcodes performed reliably across cell types and demonstrated tunability of barcoding rate which allows lineage tracking at different time scales. A compact 60-bp DAISY barcode was coupled with single-cell RNA-seq in melanoma cells, and lineages and gene expression profiles were recovered from thousands of cells. A single initial DAISY barcode could recover up to ˜700 lineages from one parent cell. Further analysis of the barcoded single-cell data revealed heritable single-cell gene expression, or transcriptional memory, and a set of high-memory genes supported by epigenetic modulation of their transcription. The optimized DAISY barcodes, while having a fraction of barcode size in comparison to Cas9 barcodes, achieved a comparable level of lineage-tracking capacity. Overall, the disclosed barcode system and components thereof provide an efficient tool for investigating cell states and dynamics in complex biological systems.

### 1. Definitions

To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2. 6.3, 6.4, 6.5, 6.6, 6.7. 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, genetics and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denbardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt′s solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a “nucleic acid” or a “nucleic acid sequence” refers to a polymer or oligomer of pyrimidine and/or purine bases, preferably cytosine, thymine, and uracil, and adenine and guanine, respectively. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506, incorporated herein by reference), locked nucleic acid (LNA; see Wahlestedt et al., Proc. Natl. Acad. Sci. U.S.A., 97: 5633-5638 (2000), incorporated herein by reference), cyclohexenyl nucleic acids (see Wang, J. Am. Chem. Soc., 122: 8595-8602 (2000), incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

A “vector” or “expression vector” is a nucleic acid carrier, such as plasmid, phage, virus, or cosmid, to which another DNA segment, e.g., an “insert,” may be attached or incorporated so as to bring about the delivery, expression, and/or replication of the attached segment in a cell.

### 2. System

The present invention relates to systems comprising proteins and nucleic acids or vectors encoding the proteins and the nucleic acids for barcoding and tracking cells. In some embodiments, the systems comprise a polynucleotide barcode flanked by two PAM (protospacer adjacent motif) sequences, or a vector encoding thereof, wherein the polynucleotide barcode comprises a first target nucleic acid sequence and a second target nucleic acid sequence; and a pair of guide RNAs (gRNAs) configured to hybridize to the two PAM sequences. The systems may further comprise a CRISPR associated (Cas) endonuclease or a nucleic acid encoding a CRISPR associated (Cas) endonuclease (e.g., Cas12a). In some embodiments, the systems comprise more than one polynucleotide barcode (e.g., two, three, four, five or more) or barcode sequence split into multiple parts by linker nucleotides. In some embodiments, the system comprises multiple sets of a polynucleotide barcode and a corresponding pair of gRNAs.

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type I, type II, or type III), and classified based on the Cas protein type and the use of a proto-spacer-adjacent motif (PAM) for selection of proto-spacers in invading DNA. Engineering CRISPR/Cas systems for use in eukaryotic cells typically involves reconstitution of the CRISPR/Cas complex. Typically, the RNA sequences necessary for CRISPR/Cas systems are referred to collectively as “guide RNA” (gRNA) or single guide RNA (sgRNA). Thus, the terms “guide RNA,” “single guide RNA,” and “synthetic guide RNA,” are used interchangeably herein and may refer to a nucleic acid sequence comprising a tracrRNA and a pre-crRNA array containing a guide sequence. The terms “guide sequence,” “guide,” and “spacer,” are used interchangeably herein and refer to the nucleotide sequence within a guide RNA that specifies the target site.

The disclosure provides systems, vectors, and nucleic acids comprising a polynucleotide barcode flanked by two PAM sequences, wherein the polynucleotide barcode comprises a first target nucleic acid sequence and a second target nucleic acid sequence. In some embodiments, the two PAM sequences are in inverse orientation relative to each other.

The terms “target DNA sequence,” “target nucleic acid,” “target sequence,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a CRISPR/Cas complex, provided sufficient conditions for binding exist. The target sequence and guide sequence need not exhibit complete complementarity, provided that there is sufficient complementarity to cause hybridization and promote formation of a CRISPR complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand.”

The target sites of the polynucleotide barcode may be flanked by a protospacer adjacent motif (PAM). For example, one target site may immediately proceed the 5′ of the first target site and one may immediately follow the 3′ end of the second target site. A PAM can be 1, 2, 3, 4, 5, 6, 7, 8, 9, 10 or more nucleotides in length. In certain embodiments, a PAM is between 2-6 nucleotides in length. In some embodiments, the PAM sequences are of inverse orientation.

Non-limiting examples of the PAM sequences include: CC, CA, AG, GT, TA, AC, CA, GC, CG, GG, CT, TG, GA, AGG, TGG, T-rich PAMs (such as TTT, TTG, TTC, TTTT (SEQ ID NO: 1057), etc.), NGG, NGA, NAG, NGGNG and NNAGAAW (W=A or T, SEQ ID NO: 1058), NNNNGATT (SEQ ID NO: 1059), NAAR (R=A or G), NNGRR (R=A or G), NNAGAA (SEQ ID NO: 1060) and NAAAAC (SEQ ID NO: 1061), where “N” is any nucleotide.

PAM sequences are often specific to the particular Cas endonuclease being used in the CRISPR/Cas complex. Cas protein PAM sequences are well-known in the art. For example, Cas12a recognizes a T-rich PAM, while Cas9 recognizes a G-rich PAM. In some embodiments, herein the PAM sequence is TTT, such that the PAM of inverse orientation is AAA.

In some embodiments the barcode further comprises a linker between the first target nucleic acid sequence and the second target nucleic acid sequence. The linker may comprise 1-20 nucleotides. In some embodiments, the linker comprises 10 nucleotide.

The polynucleotide barcode may comprise less than 200 nucleotides. In some embodiments, the polynucleotide barcode comprises less than 200 nucleotides (e.g., less than 150 nucleotides, less than 150 nucleotides). In some embodiments, the polynucleotide barcode comprises 50-60 nucleotides (e.g., 50, 51, 52, 53, 54, 55, 56, 57, 58, 59, or 60 nucleotide). In select embodiments, the polynucleotide barcode comprises 54 nucleotides.

The polynucleotide barcode may comprise any sequence which is configured to promote insertions and deletions over time. The ability of a sequence to change over time is sometimes referred to as sequence entropy. Thus, the polynucleotide barcode sequences herein as designed to have or promote high sequence entropy.

Various sequence elements or motifs may promote sequence entropy. In some embodiments, the polynucleotide barcode comprises GC directly upstream of PAM sequence at the 3′ end of the polynucleotide barcode. Thus, the final two 3′ positions of the polynucleotide barcode are GC. In some embodiments, the polynucleotide barcode comprises a cytidine at position 39 (e.g., the 39^{th} nucleotide from the 5′ end of the polynucleotide barcode sequence). In some embodiments, the polynucleotide barcode further comprises a guanosine at position 40. In some embodiments, the polynucleotide barcode comprises an adenosine at positions 45 and 46. In some embodiments, the polynucleotide barcode comprises an adenosine at position 31 and a guanosine at position 32.

In some embodiments, the polynucleotide barcode comprises CACTTG (SEQ ID NO: 1054) at positions 32-37. In some embodiments, the polynucleotide barcode comprises CCTAGTAATAG (SEQ ID NO: 1055) at positions 39-49. In some embodiments, the polynucleotide barcode comprises CCGG (SEQ ID NO: 1056) directly downstream of PAM sequence at the 5′ end of the polynucleotide barcode.

Various sequence elements of motifs may repress sequence entropy. In some embodiments, the polynucleotide barcode does not comprise a thymidine at position 54, position 49, or both. In some embodiments, the polynucleotide barcode does not comprise a guanosine at position 50 and a cytidine at position 51.

The polynucleotide barcode sequence may comprise a sequence of at least 70% similarity (e.g., at least 75%, at least 80%, at least 85%, at least 90%, at least 95%, at least 97%, at least 99%) to sequences selected from SEQ ID NOs: 1-1053. In some embodiments the polynucleotide barcode sequence comprises a sequence selected from the group consisting of SEQ ID NOs: 1-1053. In some embodiments, the polynucleotide barcode sequence is selected from the group consisting of SEQ ID NO: 1-10.

Also disclosed herein is an iterative experimental-computational method for predicting and optimizing barcode sequences based on high-throughput screening and machine-learning optimization. In some embodiments, the polynucleotide barcode is devised using the disclosed method.

The computer implemented method for designing a polynucleotide barcode sequence configured to promote insertions and deletions over time comprises: designing seed barcode sequence based on sequence elements which promote insertions and deletions, sequence elements which suppress insertions and deletions, or both; iteratively mutating the seed barcode sequence; and predicting sequence entropy as measure of insertions and deletions accumulated in a barcode sequence over time.

In some embodiments, the seed barcode may comprise less than 200 nucleotides. In some embodiments, the seed barcode comprises less than 200 nucleotides (e.g., less than 150 nucleotides, less than 150 nucleotides). In some embodiments, the seed barcode comprises 50-60 nucleotides (e.g., 50, 51. 52, 53, 54, 55, 56, 57, 58, 59, or 60 nucleotide). In select embodiments, the seed barcode comprises 54 nucleotides. During the iterative mutations, the nucleotide at each position may be changed to any of the four nucleotides individually or in combination with nucleotides at other positions to predict the sequence entropy.

Features for the barcode may be based on: one-hot-encoding of nucleotides and dinucleotides: GC or AT content information; and a Jaro-Winkler-based distance feature that encoded the process of microhomology-mediated end joining (MMEJ).

The sequence elements which promote insertions and deletions may be selected from the group consisting of: a GC dinucleotide at the 3′ end of the barcode: a CG dinucleotide starting at position 39; an AA dinucleotide starting at position 45; an AG dinucleotide starting at position 31; CACTTG (SEQ ID NO: 1054) at positions 32-37; CCTAGTAATAG (SEQ ID NO: 1055) at positions 39-49: CCGG (SEQ ID NO: 1056) at the 5′ end of the polynucleotide barcode; or a combination thereof.

Also provided herein is a system comprising a processor configured to carry out the computer implemented methods described herein.

The sequence elements which suppress insertions and deletions may be selected from the group consisting of: a thymidine at position 54; a thymidine at position 49; a GC dinucleotide starting at position 50; or a combination thereof.

The system and the nucleic acid disclosed herein may comprise a pair of guide RNAs (gRNAs) configured to hybridize to the two PAM sequences flanking the polynucleotide barcode. The gRNA may be a crRNA or a crRNA/tracrRNA (e.g., single guide RNA, sgRNA) fusion. The terms “gRNA” and “guide RNA” refer to any nucleic acid comprising a sequence that determines the binding specificity of the CRISPR-Cas complex.

The pairs of gRNAs or portion thereof that hybridizes to a target sites (e.g., the guide sequence) may be between any length. In some embodiments, the gRNAs comprise a guide sequence of less than 25 nucleotides (e.g., 5, 6, 7, 8, 9, 10, 11, 12, 13, 14, 15, 16, 17, 18, 19, 20, 21, 22, 23, 24, or 25 nucleotides.

The guide sequence of the gRNA does not need to be completely complementary to the target site. In some embodiments, the guide sequence of the gRNA is at least 50%, 55%, 60%, 65%, 70%, 75%, 80%, 85%, 90%, 95%, 96%, 97%, 98%, 99%, or at least 100% complementary to the target site. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target site (e.g., the last 5, 6, 7, 8, 9, or 10 nucleotides of the 3′ end of the target site). “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence.

To facilitate gRNA design, many computational tools have been developed (See Prykhozhij et al. (PLOS ONE, 10(3): (2015)); Zhu et al. (PLOS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer.

In addition to the guide sequence, in some embodiments, a gRNA may also comprise a scaffold sequence (e.g., tracrRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence. In some embodiments a scaffold sequence is expressed as a separate transcript. In such embodiments, the gRNA sequence further comprises an additional sequence that is complementary to a portion of the scaffold sequence and functions to bind (hybridize) the scaffold sequence.

In some embodiments, the pair of gRNAs is within a crRNA array. A crRNA array comprises multiple guide RNAs (sgRNA) derived from the fusion of CRISPR RNA (crRNA) and trans-activating crRNA (tracrRNA) expressed a single transcript, which after processing by a Cas nuclease are cleaved into separate gRNAs.

One or both of the pair of gRNAs may be a non-naturally occurring gRNA.

The Cas protein may be any Cas endonuclease. Cas protein families are described in further detail in, e.g., Haft et al., PLOS Comput. Biol., 1(6): e60 (2005), incorporated herein by reference. In some embodiments, the Cas endonuclease may be from a Class 1 (e.g., Type I, Type III, Type VI) or a Class 2 (e.g., Type II, Type V, or Type VI) CRISPR-Cas system. In some embodiments the Cas endonuclease is Cas12a, previously known as Cpf1. In some embodiments, the Cas protein is selected from Cas9, Cas12a, and Cas14.

The disclosure further provides vectors comprising encoding a Cas endonuclease (e.g., Cas12a), the polynucleotide barcode, or the pair of gRNAs. The vector comprising the Cas endonuclease may be the same or different as the vector comprising the pair of gRNAs which may be the same or different to the vector comprising the polynucleotide barcode. In some embodiments, a single vector comprises the Cas protein, the pair of gRNAs and the polynucleotide barcode. The present disclosure further provides engineered, non-naturally occurring vectors and vector systems, which can encode one or more components of the present system.

The vector(s) comprising the nucleic acid sequences encoding the Cas endonuclease, the polynucleotide barcode, and the pair of gRNAs can be introduced into a cell that is capable of expressing the polypeptide encoded thereby, including any suitable prokaryotic or eukaryotic cell.

Conventional viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present system and/or components to the cells, tissues and/or a subject. Viral vectors include, for example, retroviral, lentiviral, adenoviral, adeno-associated and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

Drug selection strategies may be adopted for positively selecting for cells comprising the nucleic acid sequences encoding the present system or components thereof.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

To construct cells that express the present system, expression vectors for stable or transient expression of the present system may be constructed via conventional methods and introduced into cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

In some embodiments, the expression of Cas12a or the Cas endonuclease is controlled by an inducible promoter. In some embodiments, the control of the expression of the Cas endonuclease allows tunability of the amount or frequency of the insertions and deletions in the polynucleotide barcode over time. For example, high concentrations of the inducing agent may promote rapid accumulation of insertions and deletions in the polynucleotide barcode sequence, whereas lower concentrations of the inducing agent may result in slow accumulation of insertions and deletions. Thus, the concentration of the inducing agent allows the accumulation in the insertions and deletions to be tunable based on speed of cell turnover, cellular growth rate, or cellular process being monitored.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability: 5′- and 3′-untranslated regions for mRNA stability and translation efficiency from highly-expressed genes like a-globin or β-globin; SV40 polyoma origins of replication and ColE1 for proper episomal replication: internal ribosome binding sites (IRESes), versatile multiple cloning sites; T7 and SP6 RNA promoters for in vitro transcription of sense and antisense RNA: a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA.

In one embodiment, the present disclosure comprises integration of the polynucleotide barcode into a recipient nucleic acid (e.g., an endogenous nucleic acid in a cell, e.g., a gene). The DNA may be packaged into an extrachromosomal, or episomal vector (e.g., AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system or components thereof may be delivered by any suitable means. In certain embodiments, the system is delivered in vivo. In other embodiments, the system is delivered to isolated/cultured cells in vitro or ex vivo to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into cells by a suitable method. Methods of delivering vectors to cells are well known in the art and may include DNA or RNA electroporation, transfection reagents such as liposomes or nanoparticles to delivery DNA or RNA: delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan 1:459(1-2):70-83), incorporated herein by reference.

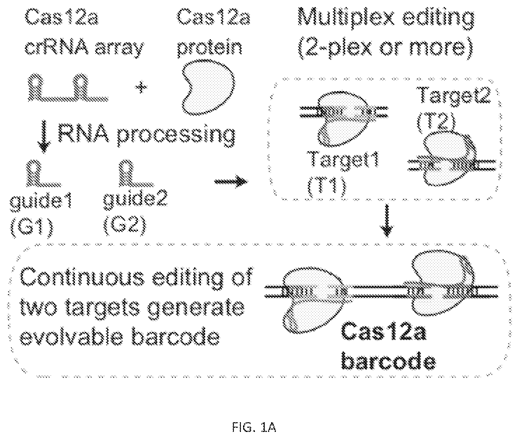

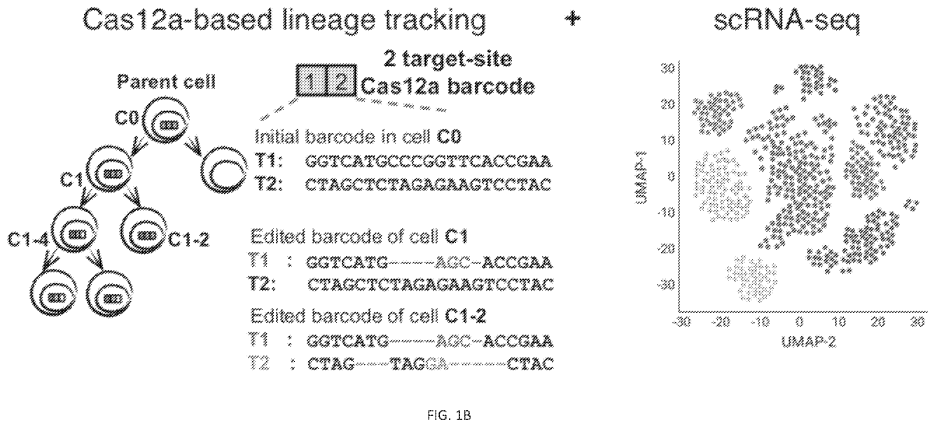

As such, the disclosure provides an isolated cell comprising the system, the vector(s) or nucleic acid(s) disclosed herein. The disclosure also provides populations of cells comprising the present systems. In some embodiments, the populations of cells comprise a distinct version of the barcode representing a particular cell generation or cell lineage (e.g., each distinct versions comprises distinct insertions or deletions within the barcode sequence (FIG. 1B)). In some embodiments, the population of cells represents up to 1000 cell generations or cell lineages (e.g., about 100, about 200, about 300, about 400, about 500, about 600, about 700, about 800 or about 900) cell generations. In some embodiments, the population of cells represents about 700 cell generations.

Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993): Lucklow, Curr. Opin. Biotechnol., 4: 564-572 (1993): and Lucklow et al., J. Virol., 67: 4566-4579 (1993), incorporated herein by reference. Desirably, the cell is a mammalian cell, and in some embodiments, the cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or Hak hamster cell lines.

In some embodiments, the cell is a cancerous cell or derived from a tumor. The cancer cell selected from the group consisting of: a lung cancer cell, a renal cancer cell, a brain cancer cell, a breast cancer cell, a pancreatic cancer cell, a colorectal cancer cell, an adrenal cancer cell, an esophageal cancer cell, a lymphoma cancer cell, a leukemia cancer cell, an acute leukemia cancer cell, a bladder cancer cell, a bone cancer cell, a bowel cancer cell, a cervical cancer cell, a chronic lymphocytic leukemia cell, a Hodgkin's lymphoma cell, a liver cancer cell, a skin cancer cell, an oropharyngeal cancer cell, a myeloma cell, a prostate cancer cell, a soft tissue sarcoma cell, a gastric cancer cell, a testicular cancer cell, a uterine cancer cell, and or a Kaposi sarcoma cell.

In some embodiments, a vector is contacted with a cell in vitro or ex vivo and the treated cell, containing the vector is transplanted into a subject.

Methods for selecting suitable mammalian cells and methods for transformation, culture, amplification, screening, and purification of cells are known in the art.

The system may further comprise at least one gRNA configured to hybridize to a recipient nucleic acid. In some embodiments, the at least one gRNA is within the crRNA array comprising the pair of gRNAs. In some embodiments, at least one or all of the gRNAs are non-naturally occurring gRNAs.

Thus, the system may be used to introduce the barcode into select locations in a recipient nucleic acid based on the specificity of the at least one gRNA. In some embodiments, the system further comprises a recipient nucleic acid.

In some embodiments, the recipient nucleic acid is a nucleic acid endogenous to a target cell. In some embodiments, the target sequence is a genomic DNA sequence. The term “genomic,” as used herein, refers to a nucleic acid sequence (e.g., a gene or locus) that is located on a chromosome in a cell. In some embodiments, the recipient nucleic acid is a gene or gene product within a target cell. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target genomic DNA sequence encodes a protein or polypeptide.

The system may further comprise components in addition to those listed, including, but not limited to: sequence tags, protein markers or marker proteins, spacers, capture sequences, and the like.

In some embodiments, the system further comprises a sequence tag configured to remain static over time. The static sequence tag may be used to uniquely identify each initial DAISY barcode sequence as it will remain unchanged over time.

In some embodiments, the system further comprises a gene editing system. The gene editing system may be a site directed gene editing system, such as a site-specific recombination-based system, zinc finger nuclease (ZFN)- or transcription activator-like effector nucleases (TALEN)-mediated gene editing system, or a CRISPR/Cas gene editing system.

In some embodiments, the gene editing system is provided on the same vectors as any or all of the gRNAs, the Cas protein, and the barcode. In some embodiments, the gene editing system is a CRISPR/Cas based system. In some embodiments, the gene editing system comprises gene editing gRNAs and, optionally, an insert or template nucleic acid.

### 3. Methods of Tracking a Cell

The disclosure also provides a methods for barcoding and/or tracking a cell. For example, the smaller barcode compared to existing barcoding strategies allows coupling of the methods described herein with existing cell-based therapies, for example to facilitate pharmacokinetic measurements or monitor cancer responses to therapeutics.

In some embodiments, the methods comprise introducing into a cell the disclosed system.

In some embodiments, the methods comprise introducing into a cell the disclosed polynucleotide barcode and a CRISPR associated protein (Cas) endonuclease or a nucleic acid encoding a CRISPR associated protein (Cas) endonuclease.

In some embodiments, the methods comprise introducing into a cell this disclosed system or the disclosed polynucleotide barcode and a CRISPR associated protein (Cas) endonuclease or a nucleic acid encoding a CRISPR associated protein (Cas) endonuclease, isolating nucleic acids from the cell at one or more time points; sequencing the polynucleotide barcode at the one or more time points: and tracking changes to original sequence of barcode in the cell at each time point.

Descriptions of the system or polynucleotide barcode in connection to the Cas proteins, the gRNAs, the polynucleotide barcode, and polynucleotides encoding thereof, set forth above in connection with the inventive system are also applicable to the methods for barcoding and/or tracking a cell. The systems, composition or vectors may be introduced in any manner known in the art including, but not limited to, chemical transfection, electroporation, microinjection, biolistic delivery via gene guns, magnetic-assisted transfection, viral or non-viral based methods, depending on the cell type as described above. In some embodiments, the one or more time points are over multiple cell generations.

In some embodiments, the methods comprise establishing lineage connections or a sequence of changes in barcode sequence between cells from different generations.

In some embodiments, the expression of Cas12a or the Cas endonuclease is controlled by an inducible promoter. In some embodiments, the control of the expression of Cas12a or the Cas endonuclease allows tunability of the amount or frequency of the insertions and deletions in the polynucleotide barcode over time. For example, high concentrations of the inducing agent may promote rapid accumulation of insertions and deletions in the polynucleotide barcode sequence, whereas lower concentrations of the inducing agent may result in slow accumulation of insertions and deletions. Thus, the concentrations of the inducing agent allow the accumulation in the insertions and deletions to be tunable based on speed of cell turnover, cellular growth rate, or cellular process being monitored.

In some embodiments, the methods further comprise adding varying concentrations of an inducing agent to the cells to vary the change rate of the original barcode sequence.

In some embodiments, the cell is a stem cell, a tumor cell, a neuron, or an adipocyte. In some embodiments, the inducing agent concentration is low when added to a tumor cell, a neuron, or an adipocyte.

In some embodiments, the cell is stem cell, cancerous cell, or intestinal epithelia. In some embodiments, the inducing agent concentration is high when added to a cancerous cell or intestinal epithelial cell.

In some embodiments, the methods further comprise at least one or all of determining single-cell transcriptomic profiles, characterizing heritability of gene expression patterns, and determining gene products which have heritable expression patterns.

Transcriptomics examines the expression level of a plurality of genes, preferably in of individual cells in a given population, by measuring the messenger RNA (mRNA) concentration. Standard techniques for single or multi-cell transcriptomics include isolation of a cell or cells from a population, lysate formation, amplification through reverse transcription and quantification of expression levels using, for example quantitative PCR or RNA-seq. Characterizing heritability of gene expression patterns and determining gene products which have heritable expression patterns can be completed by comparing gene expression profiles within barcode-defined lineage groups with an average from randomized non-lineage groups, thus allows comparisons between those genes which deviate from the group average.

In some embodiments, the barcode may be maintained as an autonomously replicating sequence or extrachromosomal element or may be integrated into host DNA. In some embodiments, the barcode integrates into genomic DNA. In some embodiments, the barcode is passed to daughter cells over multiple generations.

In some embodiments, the methods further comprise introducing mutations, insertions, or deletions within the genomic DNA (e.g., in a target gene) of a cell or population of cells. The mutations, insertions, or deletions may be the same in each cell in the population or the editing may occur randomly in each cell giving rise to a variety of modified cells. The mutations, insertions, or deletions may be introduced before, after, or at the same time as the barcode. In some embodiments, the mutations, insertions, or deletions are known disease associated genomic alterations. In some embodiments, the mutations, insertions, or deletions mediate susceptibility or resistance to treatment with a pharmacological agent.

Thus, the disclosure also provides a methods for barcoding and/or tracking mutations, insertions, or deletions, and the effects thereof, within a cell. For example, the smaller barcode and barcoding process allows coupling of the methods described herein with gene editing strategies to monitor enrichment of genomic alterations in diseased cells or drug-resistant cells or the effects of genomic alterations on drug treatment strategies for various diseases and disorders.

The mutations, insertions, or deletions may be introduced into genomic DNA using any methods known in the art. In some embodiments, the gene editing may be mediated by site-specific recombination, zinc finger nuclease (ZFN), transcription activator-like effector nucleases (TALEN), CRISPR/Cas, or other endonuclease based systems. In select embodiments, the gene editing is mediated by a CRISPR/Cas utilizing gene editing gRNAs and the Cas endonuclease introduced into the cell for the barcoding process.

The cell may be a prokaryotic or eukaryotic cell. In preferred embodiments, the cell is a eukaryotic cell. In some embodiments the cell is in vitro. In some embodiments, the cell is ex vivo.

In some embodiments, the cell is in an organism or host, such that introducing the disclosed systems, compositions, vectors into the cell comprises administration to a subject. The method may comprise providing or administering to the subject, in vivo, or by transplantation of ex vivo treated cells, systems, components, or vectors of the present system or components thereof.

In some embodiments, a plurality of cells is employed, each containing a different bar code. This can be achieved, for example, by transfecting individually isolated cells (e.g., contained in a cell array, microwell plate, microfluidic channel, or the like).

A “subject” may be human or non-human and may include, for example, animal strains or species used as “model systems” for research purposes, such a mouse model as described herein. Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing”, “administering,” “introducing,” are used interchangeably herein and refer to the placement of the systems of the disclosure into a subject by a method or route which results in at least partial localization of the system to a desired site. The systems can be administered by any appropriate route which results in delivery to a desired location in the subject.

The disclosure further provides kits containing one or more reagents or other components useful, necessary, or sufficient for practicing any of the methods described herein. For example, kits may include CRISPR reagents (Cas protein, guide RNA, vectors, compositions, etc.), transfection or administration reagents, negative and positive control samples (e.g., cells, template DNA), cells, containers housing one or more components (e.g., microcentrifuge tubes, boxes), detectable labels, detection and analysis instruments, software, instructions, and the like.

The following examples further illustrate the invention but should not be construed as in any way limiting its scope.

### EXAMPLES

### Materials and Methods

Inducible cell line generation To generate doxycycline-inducible Cas9 and Cas12a expression vectors, the following reactions were performed. First, backbone vectors containing the Tet-On 3G system (Takara Bio) were digested with AgeI (NEB), EcoRI (NEB) and BamHI (NEB), MluI (NEB), respectively. AsCas12a (iCas12a), enCas12a-HF (ienCas12a-HF), and Cas9 (iCas9) were amplified from template plasmids using primers generating homologous arms that were compatible with NEBuilderHiFi DNA Assembly (NEB) into the digested backbones containing the Tet-On 3G components (Takara) (Table 1). The Cas protein sequences were verified through primer walking of the CDS. Lentivirus was produced by co-transfecting the assembled lentiviral vectors with VSV-G envelope and Delta-Vpr packaging plasmids into HEK-293T cells, cultured at 37° C., using PEI transfection reagent (Sigma-Aldrich). Supernatant was harvested 48 hr and 72 hr after transfection. A375 cells (gift from Dr. Paul Khavari's lab) were transduced at high MOI with 8 μg/mL Polybrene using a spin-infection at 1200*g for 45 minutes. After 24 hours, cells were selected with 10 μg/mL blasticidin to establish stably expressing cell lines for inducible barcoding.

TABLE 1

| Seq | Notes |
|---|---|
| CATACGATGTTCCAGATTACGCTtgaattcgtgggaattggctcc (SEQ ID | AsCas12a/enCas12a-HF-StopEFS- |
| NO: 1062) | EcoRI-F |
| AGACAAAGGCTTGGCcatgg (SEQ ID NO: 1063) | AsCas12a/enCas12a-HF_Cas12a- |
| | StopEFS_BsiWI_R |
| CCGGCCAGGCAAAAAAGAAAAAGtgaattcgtgggaattggctcc (SEQ | Cas9_StopEFS_EcoRI_F |
| ID NO: 1064) | |
| ACTTTTGTCTTATACTTGGATCACCGGTgccaccatggc (SEQ ID | AsCas12a_F |
| NO: 1065) | |
| ccggagccaattcccacgaattcAGCGTAATCTGGAACATCGTATGGGT | AsCas12a_R |
| (SEQ ID NO: 1066) | |
| CTTTTGTCTTATACTTGGATCACCGGTgccaccatggccccaaagaag | enCas12a_F |
| Aagcggaaggtcggcagcacacagttcgagggctttac (SEQ ID NO: 1067) | |
| ccggagccaattcccacgaattcAGCGTAATCTGGAACATCGTATG (SEQ | enCas12a_R |
| ID NO: 1068) | |
| ACTTTTGTCTTATACTTGGATCACCGGTGCCACCATGG (SEQ | Cas9_F |
| ID NO: 1069) | |
| cggagccaattcccacgaattcCTTTTTCTTTTTTGCCTGGCCGG (SEQ ID | Cas9_R |
| NO: 1070) | |

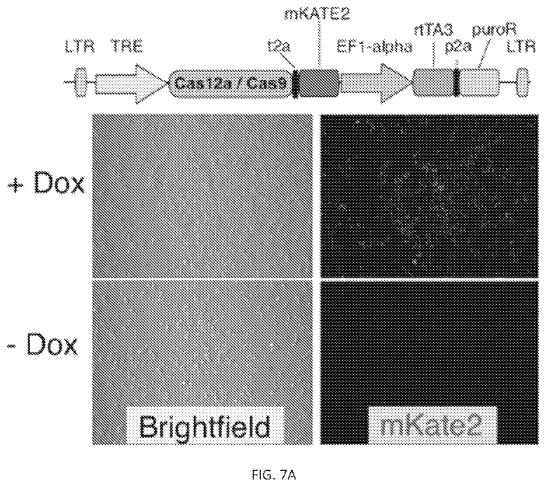

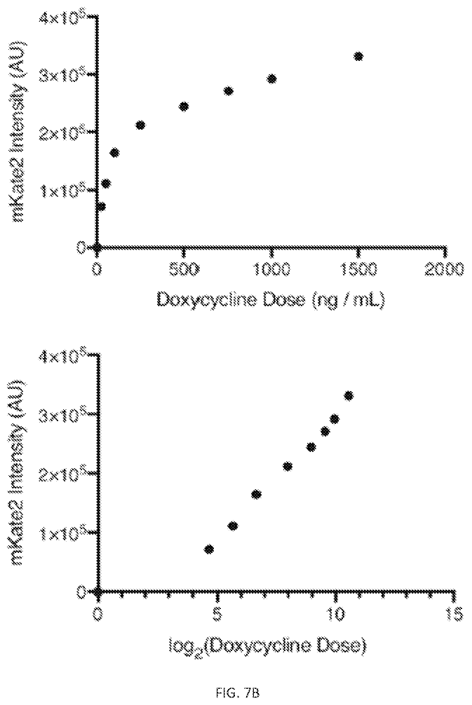

In addition, a vector was separately cloned in which AsCas12a was linked to mKate2 with a t2a element, through NEBuilderHiFi DNA Assembly (NEB). Downstream of AsCas12a and mKate2, a separate CDS containing rtTA3 linked to a puromycin resistance cassette with a p2a element was cloned. Lentivirus was produced by co-transfecting the assembled lentiviral vector with VSV-G envelope and Delta-Vpr packaging plasmids into HEK-293T cells, cultured at 37° C., using PEI transfection reagent (Sigma-Aldrich). Supernatant was harvested 48 hr and 72 hr after transfection. To generate a highly inducible A375 cell line, Cas12a expression was induced using DMEM (GIBCO) supplemented with penicillin/streptomycin, 10% Inactivated Fetal Calf Serum, and 400 ng/ml doxycycline (Sigma-Aldrich) and sorted the top 10% of mKate2-positive cells. As a “2A-mKate” fluorescent reporter protein was fused with the Cas enzymes, their inducible expression was monitored and validated via imaging (FIG. 7). These cell lines supported doxycycline-dependent gene-editing at endogenous human genomic locus with minimal leakage (FIG. 8).

Barcode design and cloning A published Cas9 barcode sequence (Bowling, S. et al. Cell 181, 1410-1422.e27 (2020)) was modified in two ways. First, to make the sequence more compact, two target barcode designs were assembled in an arrayed format (three in total). Second, a Cas12a PAM was appended in front of each target sequence, thereby allowing direct Cas9 and Cas12a editing comparisons within a synthetic barcode locus (Table 2). To clone the Cas9 gRNAs, a gBlock gene fragment (IDT) containing gRNA1, scaffold sequence, mU6 and gRNA2 was ordered. The fragment was cloned into a Esp3I digested lentiviral vector containing a hU6 promoter from which crRNAs can be expressed using NEBuilder HiFi DNA Assembly (NEB). The Cas12a crRNA array and target array were cloned through OE-PCR using Phusion Flash High-Fidelity PCR Master Mix (ThermoFisher Scientific) followed by NEBuilder HiFi DNA Assembly (NEB) according to the manufacturer's instructions. Constructs were verified by Sanger sequencing using a U6 sequencing primer and a WPRE sequencing primer. Lentivirus was produced by co-transfecting the library with VSV-G envelope and Delta-Vpr packaging plasmids into HEK-293T cells, cultured at 37° C., using PEI transfection reagent (Sigma-Aldrich). Supernatant was harvested 48 hr and 72 hr after transfection.

Barcode lentiviral delivery A375 cells stably expressing iCas12a, ienCas12a-HF, and iCas9 were transduced at high MOI with the barcodes as described above. 72 hours after transduction, 1e5 cells were collected for gDNA extraction using Quick Extract (Lucigen). Doxycycline was added to the media of the remaining cells at the following doses: 0 ng/mL, 10 ng/mL, and 1000 ng/mL. Cells were collected 2 days after doxycycline induction for gDNA extraction. Barcodes were amplified with primers containing Illumina adapters using NEB Ultra II Q5 Master Mix (NEB) according to the manufacturer's instructions (Table 2). Paired-end reads (150 bp) were generated on an Illumina MiSeq with a 75k read depth per sample.

TABLE 2

| Seq | Notes |
|---|---|
| TTTGGACTGCACGACAGTCGACGATGGTTTCGACACGACTCGCGCATACGATGG | Barcode 1 |
| (SEQ ID NO: 1071) | |
| TTTGGACTACAGTCGCTACGACGATGGTTTCGCGAGCGCTATGAGCGACTATGG | Barcode 2 |
| (SEQ ID NO: 1072) | |
| TTTGGATACGATACGCGCACGCTATGGTTTCGAGAGCGCGCTCGTCGACTATGG | Barcode 3 |
| (SEQ ID NO: 1073) | |
| TCGTCGGCAGCGTCAGATGTGTATAAGAGACAG tatgtgtgggagggctaaac (SEQ ID | NGS primer_for |
| NO: 1074) | |
| GTCTCGTGGGCTCGGAGATGTGTATAAGAGACAG tgtaatccagaggttgattgtcg (SEQ ID | NGS primer_rev |
| NO: 1075) | |

Barcode bioinformatic analysis Targeted barcode deep sequencing data was analyzed using a custom pipeline. Briefly, reads were split into reads 1 and 2 and then merged using flash v1.2.11 with default parameters. Next, barcode sequences were demultiplexed by computing the minimum Levenshtein distance min(leva,b(|a|, |b|) where a is the read sequence and b is the reference sequence. Reads were assigned to the barcode with the minimum Levenshtein distance. Next, reads were aligned to their reference as described above using NEEDLEALL. Finally, the information content at each time point contained within the target sites across doxycycline conditions was computed as the Shannon entropy:

$$H ( X ) = - {\sum }_{i = 1}^{n} P ( {x}_{i} ) log P ( {x}_{i} )$$

where xᵢ is a unique editing outcome generated by either Cas12a or Cas9.

Direct comparison of Cas9 and Cas12a endogenous genome editing Three genomic loci were identified that contained both a Cas12a and Cas9 PAM sequence within the AAVS1, CCR5, and DNMT1 genes. Cloning of gRNAs (Table 3) was performed with BbsI or Esp3I (New England Biolabs) through a Golden Gate assembly approach into either a Cas12a expressing backbone or a Cas9 expressing backbone, respectively. Constructs were sequence verified by Sanger sequencing using a U6 sequencing primer: 5′-GACTATCATATGCTTACCGT-3′(SEQ ID NO: 1076). HEK293T cells (7e4) were transfected using Lipofectamine 3000 (ThermoFisher Scientific) in 48-well plates. Genomic DNA was extracted from transfected cells 72 hours later using QuickExtract (Lucigen). The targeted loci were then amplified using Phusion Flash High-Fidelity PCR Master Mix (ThermoFisher Scientific) according to the manufacturer's instructions with primers containing Illumina sequencing adapters (Table 3). Paired-end reads (150 bp) were generated on an Illumina MiSeq platform.

TABLE 3

| Seq | Target | Nuclease | Notes |
|---|---|---|---|
| Tgcttacgatggagccagag (SEQ ID NO: 1077) | AAVS1 | Cas9 | sgRNA sequence |
| Cttacgatggagccagagaggat (SEQ ID NO: 1078) | AAVS1 | Cas12a | sgRNA sequence |
| TAGAGCTACTGCAATTATTC (SEQ ID NO: 1079) | CCR5 | Cas9 | sgRNA sequence |
| GCCTGAATAATTGCAGTAGCTCT (SEQ ID NO: 1080) | CCR5 | Cas12a | sgRNA sequence |
| TTTCCCTTCAGCTAAAATAA (SEQ ID NO: 1081) | DNMT1 | Cas9 | sgRNA sequence |
| TTTCCCTTCAGCTAAAATAAAGG (SEQ ID NO: 1082) | DNMT1 | Cas12a | sgRNA sequence |
| CCATCTCATCCCTGCGTGTCTCC gtgttcagtctccgtgaacg | | | DNMT1-3_NGS_for |
| (SEQ ID NO: 1083) | | | |
| CCTCTCTATGGGCAGTCGGTGATg aagtcactctggggaacacg | | | DNMT1-3_NGS_rev |
| (SEQ ID NO: 1084) | | | |
| CCATCTCATCCCTGCGTGTCTCC ggtgacacacccccatttcc | | | AAVS1_NGS_for |
| (SEQ ID NO: 1085) | | | |
| CCTCTCTATGGGCAGTCGGTGATg actgagaaccgggcaggtca | | | AAVS1_NGS_rev |
| (SEQ ID NO: 1086) | | | |
| CCATCTCATCCCTGCGTGTCTCC tctcttctgggctccctaca | | | CCR5-9_NGS_for |
| (SEQ ID NO: 1087) | | | |
| CCTCTCTATGGGCAGTCGGTGATg agcagtgcgtcatcccaaga | | | CCR5-9_NGS_rev |
| (SEQ ID NO: 1088) | | | |

Targeted deep sequencing data was analyzed using a custom pipeline. Briefly, reads were split into reads 1 and 2 and then merged using flash v1.2.11 with default parameters. Next, merged reads were aligned to their respective reference genomic locus using NEEDLEALL with the following parameters: needleall-asequence<reference>-bsequence<query>-gapopen 10-gapextend 0.5-aformat3 sam The resulting sam files were then filtered to remove reads with flanking insertions or deletions. The filtered editing outcomes were then used to quantify the Shannon entropy of edits at each locus as described above.

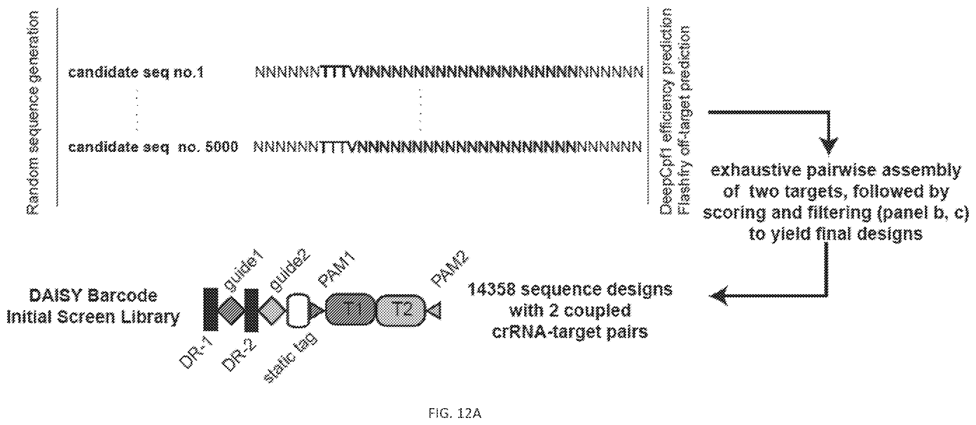

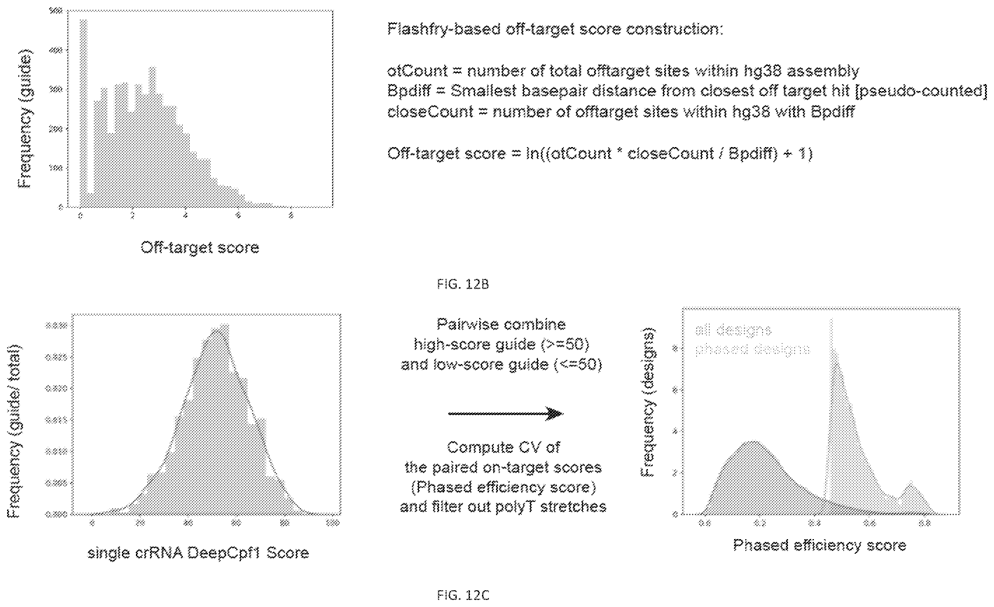

crRNA and target paired library (DAISY barcode library) A library of 5000 random Cas12a targets was generate and filtered for low off-target activity, GC content, and polyT stretches using FlashFry. Target regions were then scored for their predicted indel efficiency for each target region using DeepCpf1. A custom script was then used to partition a target region into an efficient editing category (DeepCpf1 score≥50) and an inefficient editing category (DeepCpf1 score<50) and to create all pairwise combinations of efficient and inefficiently targeted sequences. Next, all pairwise combinations were scored for their average efficiency score and standard deviation of efficiency scores to create a composite editing score for the combined pair. The coefficient of variation of the composite editing score was computed and used to rank all pairwise combinations. The top 55^{th} percentile target regions were chosen (14358 unique sequences) to assemble into a final pooled oligonucleotides (Twist Biosciences). As negative controls, 12 barcode sequences in which the spacer and target sequences were mismatched were included. Each DAISY barcode sequence within the 14358 pool was uniquely identified with a 10 bp sequence as static tag (FIG. 12).

For the second screen using an optimized DAISY barcode library, the CLOVER model was utilized to identify 2000 barcode sequences predicted to have increased barcode diversity. Briefly, seed barcode sequences were iteratively mutated to explore the diverse sequence space. 10 iterations were performed to identify the final set of 2000 barcode sequences. The 60 nucleotide barcode sequences were then synthesized along with their paired crRNAs and a unique 10 bp NGS tag sequence as above.

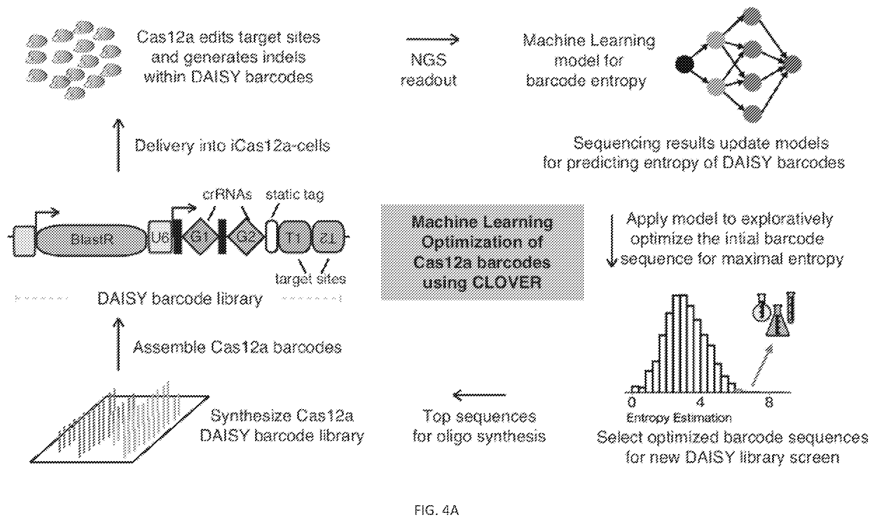

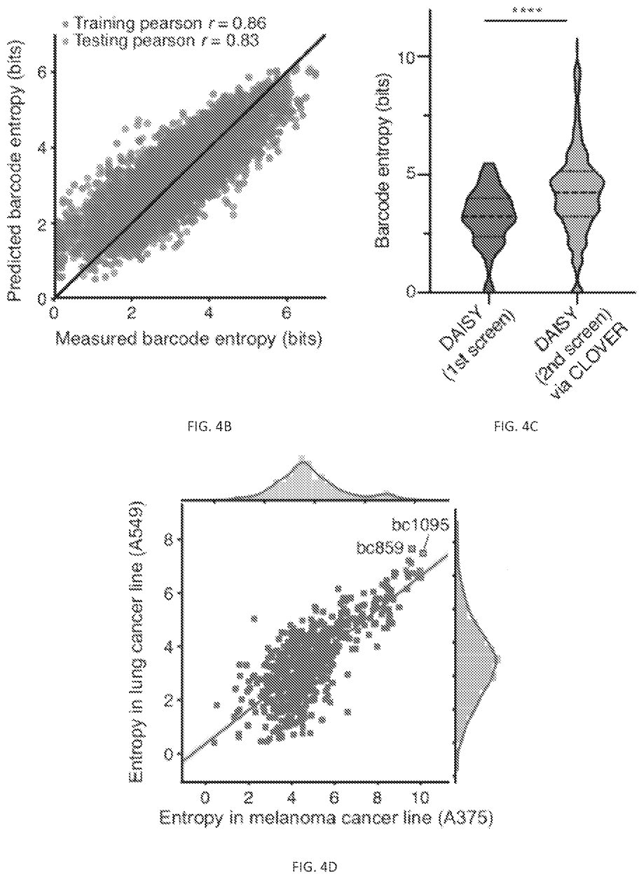

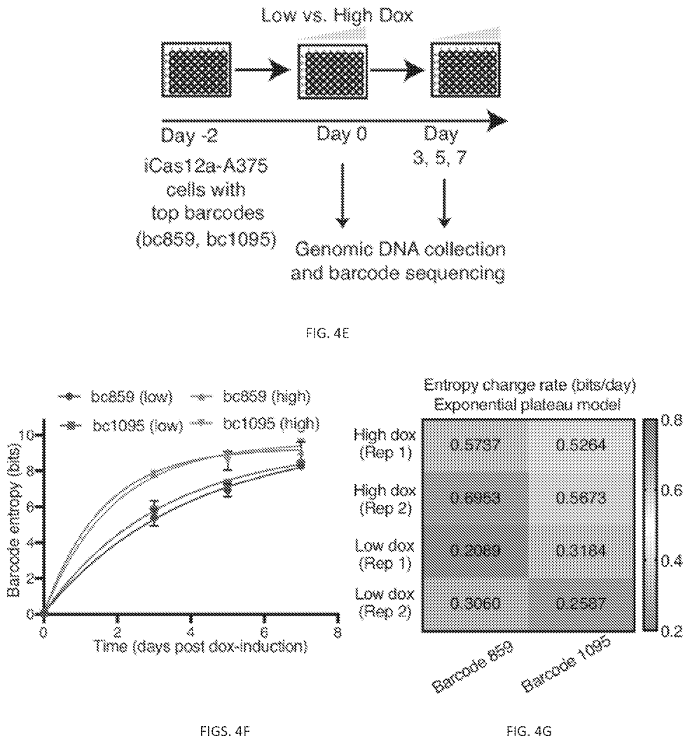

Library Cloning A lentiviral vector was constructed using HiFi DNA Assembly Master Mix (NEB) to remove the existing Cas9 scaffold sequence and to incorporate two Esp3I (NEB) restriction sites downstream of an AsCas12a direct repeat sequence (FIG. 4A). The resulting vector was digested with Esp3I (NEB) at 37° C. for 1 h and gel purified with Monarch DNA Gel Extraction Kit (NEB). Oligonucleotides from Twist Bioscience, with dual crRNAs paired with their targets, were resuspended to 10 ng/uL. The oligonucleotide pool was PCR amplified using KAPA Biosystems HiFi HotStart ReadyMix (2×) and gel extracted from a 2% agarose gel (Table 4). The amplified library was then assembled into the library backbone using HiFi DNA Assembly Master Mix (NEB) with a molar ratio of 20:1, respectively. The assembly reaction was then precipitated and resuspended in TE (Macherey-Nagel) according to a previously published protocol (Joung, J. et al. Nat.Protoc. 12, 828-863 (2017)). The entire reaction was used to transform Endura Electrocompetent Cells (Lucigen) following the manufacturer's protocol. The transformed cells were cultured at 25° C. for 48 hours to minimize recombination between direct repeats. Colonies were then harvested directly and plasmid DNA was extracted with a Plasmid Plus Maxi Kit (Qiagen).

Sequencing Library generation Genomic DNA was extracted with DNeasy Blood & Tissue Kit (Qiagen) following the manufacturer's protocol. A first round PCR reaction was performed using 300 ng of genomic DNA, 0.2 μl 100 mM primer and 25 μl NEBNext Ultra II Q5 Master Mix (NEB) per reaction. A total of 12 reactions were performed per sample to ensure adequate representation of the lentiviral pool. PCR reactions were performed according to the following conditions: 98° C. 30 s, followed by 25 cycles of 98° C. 10 s, 60° C. 20 s, 72° C. 20 s, followed by 72° C. 2 min. The PCR product was pooled and cleaned with 0.8×CleanNGS DNA SPRI Beads (Bulldog Bio) and resuspended in 20 μl of elution buffer. The resulting purified PCR products were then quantified using a Take3 Plate Reader (BioTek) and 10-20 ng were loaded into a second round of amplification using NEBNext Q5 Master Mix (NEB) to incorporate flow-cell adaptor sequences and sample indexes to enable demultiplexing of pooled samples (Table 4). The PCR reaction was performed with the following conditions: 98° C. 30 s, followed by 15 cycles of 98° C. 5 s, 72° C. 20 s. The resulting libraries were then cleaned with 0.8×CleanNGS DNA SPRI Beads (Bulldog Bio), equimolarly pooled, and then gel-extracted. The resulting pooled library was sequenced on an Illumina HiSeq 4000 sequencer using paired-end 150 cycle reads.

TABLE 4

| Seq | Notes |
|---|---|
| CGTAATTTCTACTCTTGTAGAT (SEQ ID NO: 1089) | forward primer to amplify |
| | oligo pool |
| ATCCAGTTTGGTTAATTTAATTA (SEQ ID NO: 1090) | forward primer to amplify |
| | oligo pool |
| ATCTTGTGGAAAGGACGAAACACCGTAATTTCTACTCTTGTAGAT | pN28 construction |
| GGAGACGGTTGTAAATGAGCAC (SEQ ID NO: 1091) | |
| TACGCCTTAATTAAAAA AGAGACGTACAAAAAAGAGCAAGAAG | pN28 construction |
| (SEQ ID NO: 1092) | |
| GTCTCTTTTTTAATTAA GGCGTAACTAGATCTTGAGACAAATG | pN28 construction |
| (SEQ ID NO: 1093) | |
| TTATCCATCTTTGCACCCGG (SEQ ID NO: 1094) | pN28 construction |
| CCATCTCATCCCTGCGTGTCTCC CGTAATTTCTACTCTTGTAGAT | forward NGS primer |
| (SEQ ID NO: 1095) | |
| CCTCTCTATGGGCAGTCGGTGATg ATCCAGTTTGGTTAATTTAATTA | reverse NGS primer |
| (SEQ ID NO: 1096) | |

Sequencing processing pipeline A total of 8 samples were sequenced on an Illumina HiSeq 4000. These paired-end reads were first demultiplexed by their 10 bp amplicon barcode sequence, then parallel aligned to their amplicon barcode-assigned reference sequence using the EMBO needleall software with the following scoring matrix: match=5, mismatch=−4, gap-open=−20, gap-extension=−0.5, where mismatch penalties for Ns, Vs and Bs were set to 0. The generated indel profiles for the sample collected at Day 0 (day of doxycycline-induction) were used to filter out indels observed in samples collected at later time points. To enable comparison of barcodes with variable read depth, barcodes were down-sampled such that all barcodes were uniformly represented with 500 reads. The resulting indel profiles were used to define the mutational outcomes of Cas12a nuclease activity using the Shannon entropy (barcode diversity) as previously described.

For benchmarking versus publicly available barcode datasets that used Cas9, the following datasets were downloaded: GEO: GSE146972 and GEO:GSE81713 that contained fastq sequencing data. These datasets were aligned to their barcode references. Resulting alignments were parsed to associate indels with each target site. More specifically, the beginning of an indel determined its target assignment within the barcode. The information content within each target was computed as described above to determine the information content. Each target site was pairwise assembled to generate estimates of how two-target barcodes would perform. Similarly, with the top DAISY barcodes, the cigar string was parsed to assign indels to each target. The total barcode information content was computed as the sum of each target's information content.

Feature space design The problem of finding the highest-entropy barcodes was embedded in a Bayesian optimization framework. As input to the Bayesian model, representative features are necessary to capture the characters of CRISPR barcode that contribute to difference in editing outcomes thus in entropy. A 4906-dimensional feature space (that includes both nucleotide-based and microhomology-based information was designed. Single-nucleotide features and Dinucleotide features were included in the nucleotide-based features concerned with the 60bp target region (common base pairs in PAMs not included). Since spacers matched the targets, the varying lengths of the two guides from spacers were added as another feature (21, 23 and 25 nt). For the two subsequences flanking the deletion part, considering that the base pairs closest to the cutting site take on a comparatively heavier responsibility for MMEJ-based deletion, the Jaro-Winkler distance was used, which gives more weight to the common prefix of two sequences, of this two subsequences to represent their level of homology. The proportion of GC-base pairs in these two subsequences was calculated to be in the feature space. There was a total possibility of 53*52/2 deletions (base pairs in PAMs not considered). For each deletion, the two 15-bp (or less, depending on the length of the deletion as well as if there are enough base pairs flanking it)-subsequence-pairs flanking both the left and right sides of the deletion were considered. This led to a total of 960*2*2 (3840) microhomology-based features.

Ridge regression on principal components The full barcodes dataset was divided into a 70%-training set and a 30%-testing set. Principal Components Analysis was implemented on the training set to find directions of the feature space with high variances. To predict the entropy, a Ridge regression model was trained based on the 500 principal components with highest explained variances. A 5-fold cross validation was used to pick the penalty.

Machine learning optimization of barcode sequences using upper confidence bound (UCB) method In each round t, the agent picks an arm Xₜ from a given decision set Ωₜ⊂R^{d}. Subsequently she receives a reward—Yₜ=f(Xₜ)^{T}·θ_{*}+ηₜ—modeled as a linear transform of f(Xₜ) where f:R^{d}→Rⁿ is a transform function, θ_{*}∈Rⁿ is an unknown parameter and θₜ∈Rⁿ is a random noise satisfying zero mean E[ηₜ|X_{1:t}, η_{1:t-1}]=0 and conditional R-sub-Gaussian

$$E [ {e}^{r η t} \vert {X}_{1 : t} , {η}_{1 : t - 1} ] \leq exp ( \frac{{r}^{2} - {R}^{2}}{2} ) ,$$

∀r∈R. The agent seeks x*=argmax_{x∈Ω}_{ₜ}f(x)^{T}θ_{*} to maximize the instantaneous reward.

The upper confidence bound (UCB) method solves this problem by following the optimism in the face of uncertainty (OFU) principle. Since θ_{*} is unknown, one has to estimate it, however best estimation based on current information might stress too much on exploitation and lead to local optimal. The OFU principle addresses the trade-off between exploitation and exploration by constructing a confidence ellipsoid for θ_{*}. This confidence ellipsoid can be deduced using concentration inequality. Denote f(X_{1:t})=(f(X₁)^{T}, . . . , f(Xₜ)^{T} and Y_{1:t}=(Y₁, . . . , Tₜ)^{T}. Let \hat{θ}ₜ & to be the estimator of θ_{*} returned by a ridge regression with penalty λ on

$$f ( {X}_{1 : t} ) , {Y}_{1 : t} , i . e . {\hat{θ}}_{l} = arg {max}_{θ} ‖ f ( {X}_{1 : t} ) \cdot θ - {Y}_{1 : t} ‖ \frac{2}{2} + λ ‖ θ ‖ \frac{2}{2} = {( {f ( {X}_{1 : t} )}^{T} f ( {X}_{1 : t} ) + λ I )}^{- 1} {f ( {X}_{1 : t} )}^{T} {Y}_{1 : t}$$

Assume that S, L satisfy ∥θ_{*}∥≤S and for all t≥1, ∥Xₜ∥₂≤L. Then for any δ>0, with probability at least 1−δ, for all t≥0, θ_{*} lies in

$${C}_{t} = \{ θ ∈ {R}^{n} : {‖ {\hat{θ}}_{t} - θ ‖}_{{V}_{T}} \leq R \sqrt{n ln ( \frac{1 + {tL}^{2} / λ}{δ} ) + {λ}^{\frac{1}{2}} S} Where {V}_{t} = λ I + {f ( {X}_{1 : t} )}^{T} f ( {X}_{1 : t} ) and {‖ {\hat{θ}}_{t} - θ ‖}_{{V}_{t}} = {( {\hat{θ}}_{t} - θ )}^{T} {V}_{t} ( {\hat{θ}}_{t} - θ ) .$$

The UCB methods picks the arm

$${x}^{*} = arg {max}_{x ∈ {Ω}_{t} , θ ∈ {C}_{t}} {f ( x )}^{T} \cdot θ$$

In this setting, Ωₜ is the set contains all DAISY designs, f is the transform function that combinate the feature design as well as the PCA step to process the sequence into the input of online learning and Y is the entropy. At each round, the top m designs that maximize the objective function above were picked.

scDAISY-seq vector construction The top performing barcodes were synthesized to include the following components: (1) the crRNA targeting the evolvable molecular barcode (82 nt) (2) the evolvable molecular barcode targets (60 nt) (3) a static tag (10 nt) and (4) a 10×Genomics capture sequence (22 nt). These components were cloned downstream of the hU6 promoter through Esp3I digestion (NEB) followed by Gibson assembly using HiFi DNA Assembly Master Mix (NEB). The 10×Genomics capture sequence (22 nt) allows for binding of the expressed molecular barcode directly to 10×Genomics gel beads contained within the Chromium Next GEM Single Cell 3′ Reagent Kit v3.1.

Lentiviral delivery of barcodes to mammalian cells for single cell barcoding Barcode vectors were lentivirally packaged according to the aforementioned protocol. Resulting lentivirus was then used to transduce at least 5e4 A375 cells harboring doxycycline-inducible AsCas12a. Cells were then bottlenecked through limited dilution to contain ˜5 clones/well in a 96-well plate. Upon seeding, Cas12a was induced with 400 ng/ml dox in conditioned DMEM. Cells were then expanded for approximately two weeks for barcode editing. After two weeks cells were harvested for single-cell RNA-sequencing using the Chromium Next GEM Single Cell 3′ Reagent Kit v3.1 under the manufacturer's protocol unless otherwise noted.

scDAISY-seq barcode recovery sequencing and analysis The cDNA library from step 4.2 of within the manufacturer's protocol (10×genomics), was used as a template for PCR. A forward primer, binding to the Nextera (Illumina) read 1 sequence, was used with a reverse primer that binds specifically to the expressed barcode sequence at the terminal direct repeat (Table 5). The PCR was performed with NEB Ultra II Q5 Polymerase following the manufacturer's protocol. Barcode libraries were sequenced with paired end 150 cycle configuration on a MiSeq instrument (Illumina). Resulting fastq data were processed using CellRanger Count v4 (10×Genomics) using a custom feature barcode reference. The resulting .bam file was filtered to include only reads mapping to the custom feature barcode reference. Next, reads were collapsed into groups defined by their 10×Cell Barcode and UMI sequences. The collapsed reads were then parsed to extract only the evolvable barcode sequence and aligned to the reference evolvable barcode sequence using the Smith-Waterman algorithm with the following parameters: gapopen=13, gapextend=0.5. The alignment .bam file was then parsed to group the Cas12a-edited barcode alignment to a 10×Cell Barcode and UMI sequence—corresponding, in theory, to a unique transcript within a single cell. Cells were then clonally grouped into lineage groups based upon their static barcode sequence. The phylogeny of cells within each lineage group was reconstructed using the Neighbor Joining algorithm5.

TABLE 5

| Seq | Notes |
|---|---|
| TCGTCGGCAGCGTCAGATGTGTATAA (SEQ ID NO: | Forward primer off of |
| 1097) | cDNA template |
| GTCTCGTGGGCTCGGAGATGTGTATAAGAGACA | Reverse primer |
| gTTTCTCCTCTCGGAGATTTGC (SEQ ID NO: 1098) | (specific to DAISY cassette) |

Transcriptional memory analysis Single-cell transcriptome profiles were generated from libraries sequenced with paired end 150 cycle configuration on a HiSeq 4000 (Illumina). Resulting fastq data were processed using CellRanger Count v4 (10×Genomics) using a custom feature barcode reference. Gene expression data were processed using scanpy. Briefly, barcoded cells were selected from the raw gene expression matrix. Cells were filtered based upon their UMI abundance (100 UMI cutoff) and expression frequency (expressed in at least three cells). The data were further filtered to exclude cells in which mitochondrial genes represented greater than or equal to 20% of the total UMIs within the cell. Finally, the UMI counts were normalized on a per cell basis with the target sum set to le4 (transcripts/1e4 molecules). Finally, the normalized counts were pseudo-counted by 1 and logarithmically transformed. The phylogenetic structure of each lineage group was used to assemble a dictionary in which all leaves within the tree were uniquely grouped by their most recent common ancestor (MRCA) into a sublineage (s). For each gene (g), a memory index (m) was calculated as follows:

Let μₛ be the mean expression of g within s

Let σₛ be the standard deviation of the expression of g within s, where

$$C {V}_{s} = \frac{{σ}_{s}}{{μ}_{s}}$$

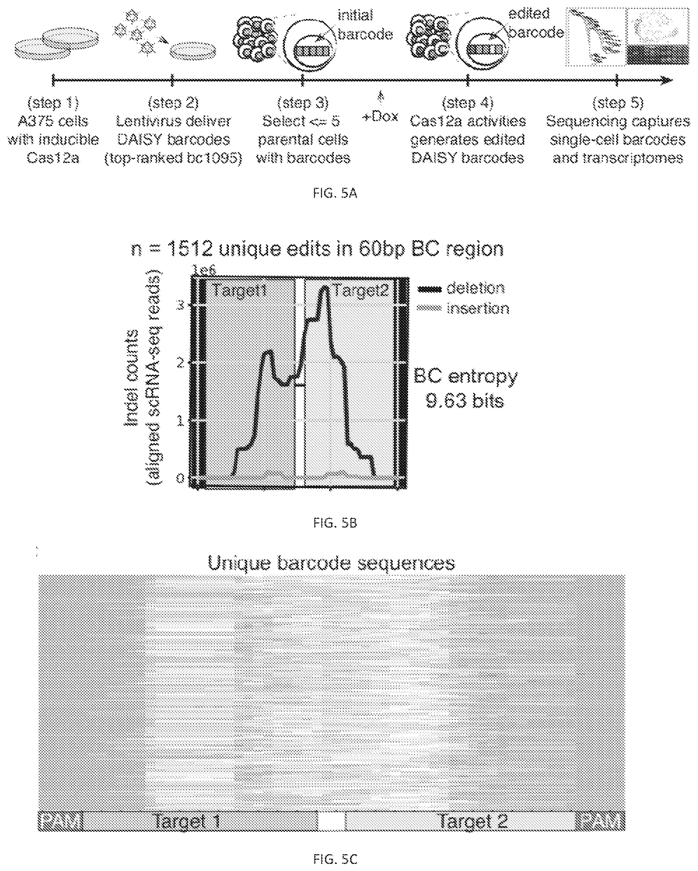

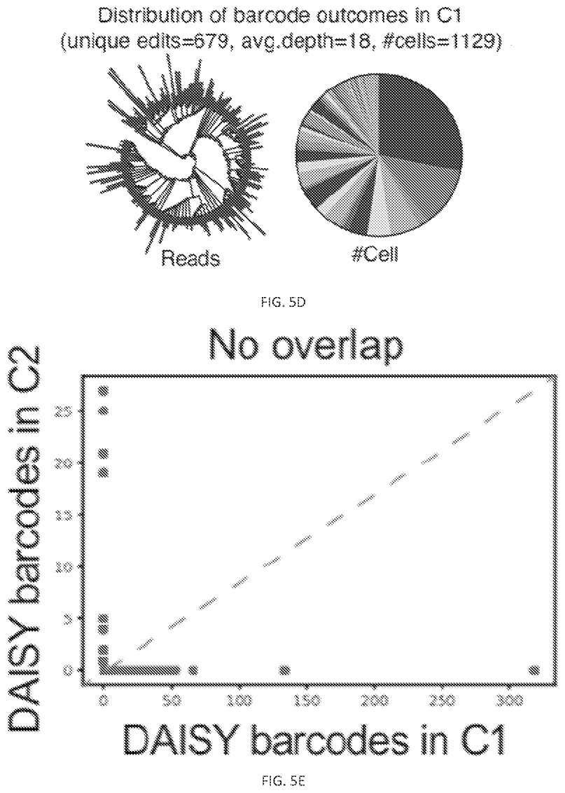

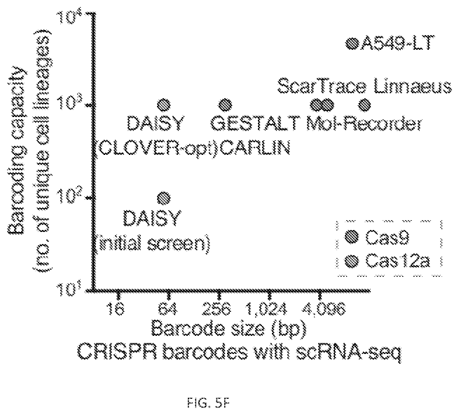

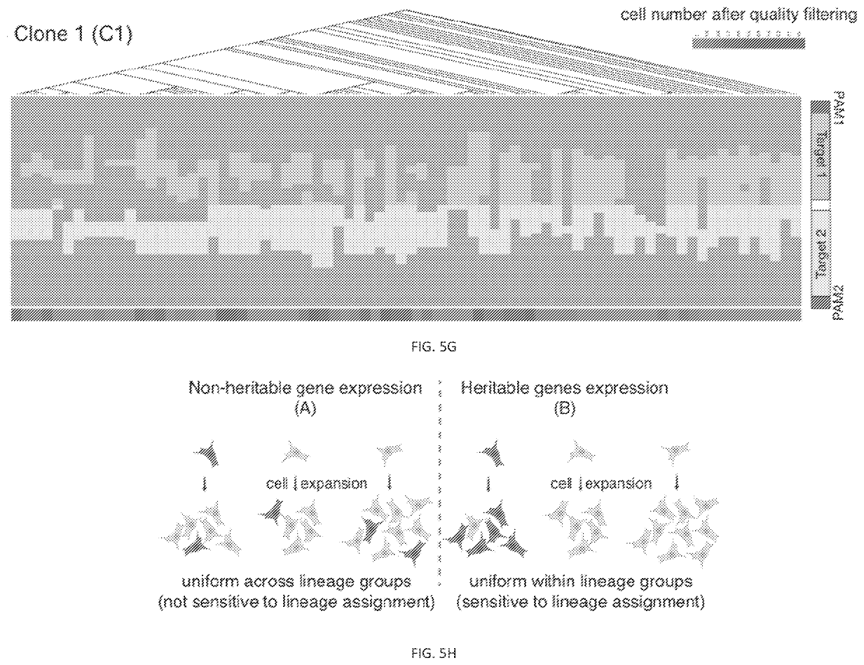

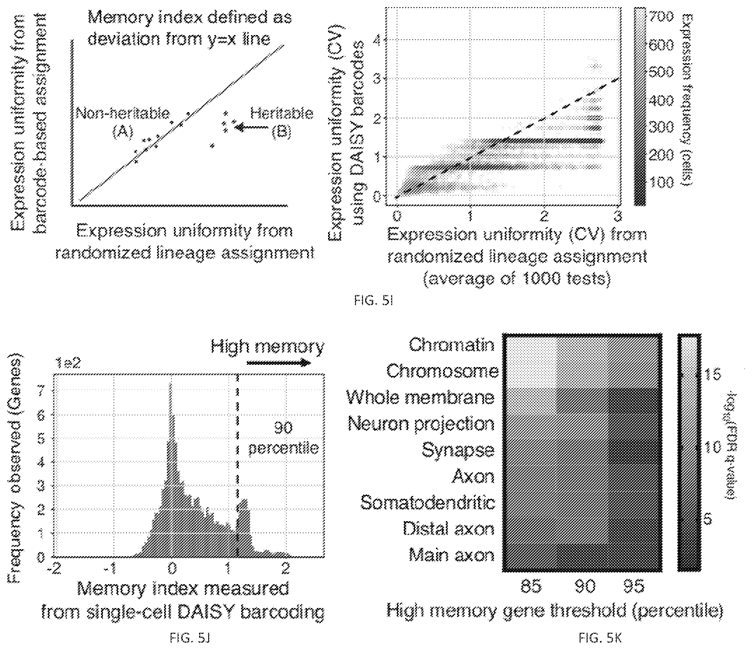

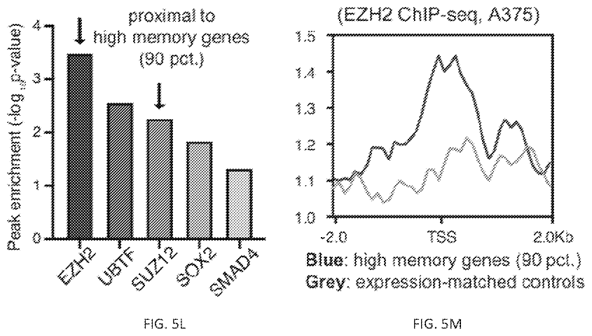

Across all sublineages (S), min (CVₛ) was determined. To generate the null distribution, 1000 random phylogenetic trees were simulated with the same sublineage sizes as in the C1 tree (FIG. 5E). The minimum CV within a random sublineage min(CV_{random}) was computed as described above. The mean min(CV_{random}) was determined across all simulations. The final memory index was defined as:

$$m = mean ( min ( {CV}_{random} ) ) - min ( {CV}_{s} )$$

In addition, the Gini coefficient and average expression level within a sublineage were also computed across all sublineages (S) and genes that were expressed in A375 cells. Memory genes across three percentile thresholds (85th, 90th, and 95th) were selected for functional follow-up with gene set enrichment analysis. Briefly, a publicly available GSEA software portal was utilized in which each memory gene set for significantly enriched Gene Ontology (GO) biological components was queried. Second, the enrichR package was utilized to identify proteins with enriched ChIP-Seq peaks from publicly available ENCODE data. To follow-up publicly available ChIP-seq data from A375 cells (GSE133834) was downloaded. Genes with a memory index greater than or equal to the 90th percentile (n=1001 genes) were selected for visualization of EZH2 binding profiles using deeptools2. To generate the TPM-matched gene set, bulk RNA-expression levels were determined for each memory gene in A375 cells using the CCLE12. Genes whose values were within +/−20% of memory gene expression were included for visualization. Briefly, BED files containing the transcription start sites (TSSs) were generated for all memory and control gene sets and used to compute a matrix in which genomic regions are scored for enrichment in EZH2 binding. The intensity of EZH2 binding was visualized using the plotheatmap function within deeptools2.

TCGA analysis RNA-Seq profiles of skin cutaneous melanoma (SKCM) lesions were downloaded from The Cancer Genome Atlas (TCGA). The Shannon entropy was then calculated using the expression levels of each gene across all tumors to generate a transcriptional heterogeneity metric for each tumor. The EZH2 expression level within each tumor was determined and the set of values was binned into four groups for visualization of the relationship between the expression level of EZH2 in a tumor and its transcriptional heterogeneity. To benchmark the relationship between EZH2 expression level and transcriptional heterogeneity, the Spearman rank correlation coefficient (SCC) of the linear relationship between the expression level of each gene within a tumor and the transcriptional heterogeneity of the tumor was calculated. Genes were ranked by the SCC and plotted.

### Example 1

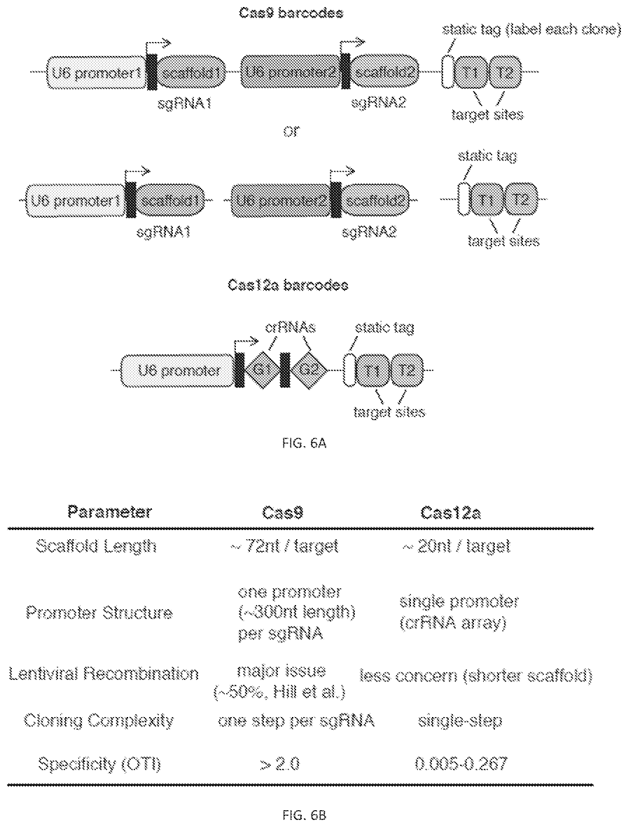

Cas12a, a class II type V CRISPR-Cas enzyme, bas dual RNase and DNase activities. Cas12a can bind to the ˜20 palindromic nucleotides scaffold sequences (often termed direct repeat, DR) and process a crRNA array to generate multiple crRNAs. The processed crRNAs bear distinct guide sequences that allow Cas12a to edit multiple target sites matching the guide (FIG. 1A). This multi-target property of Cas12a allows a substantially more compact barcode design compared to Cas9 (FIG. 6). Moreover, prior studies demonstrated that Cas12a had higher cleavage specificity than Cas9, which could reduce toxicity from off-target cutting in barcoding experiments (FIG. 6). As Cas12a editing happens over time, the initial barcode sequence evolves and branches into multiple lineages while the cell population expands (FIG. 1B, left). When coupled with single-cell RNA-Seq (scRNA-Seq), the evolvable barcodes enable reconstructing a population lineage tree from readouts of edited barcodes (FIG. 1B).

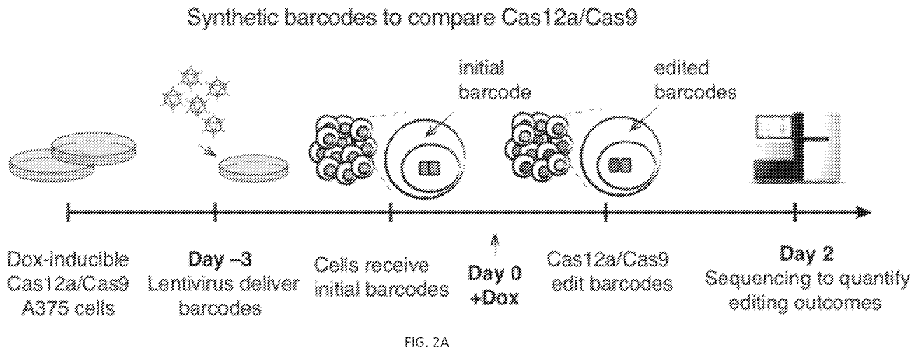

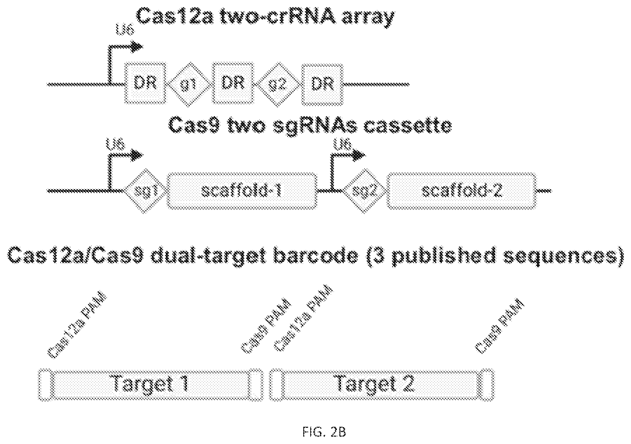

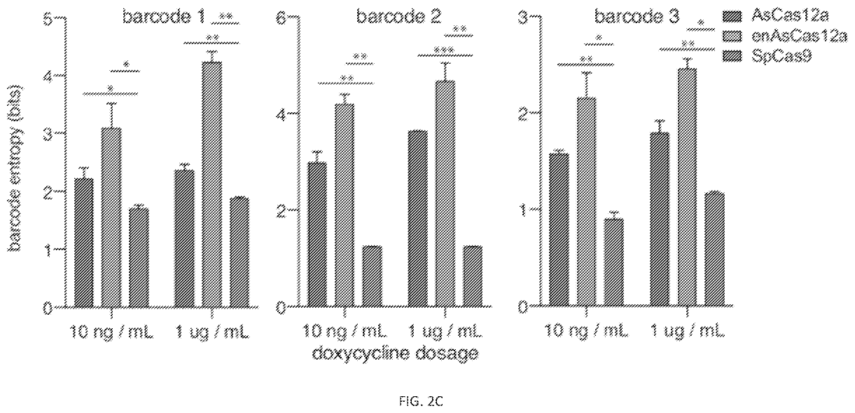

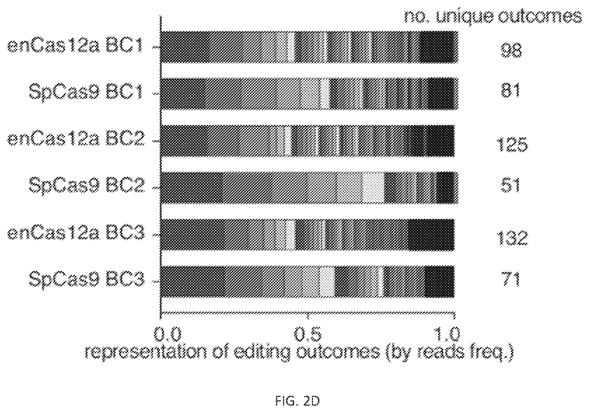

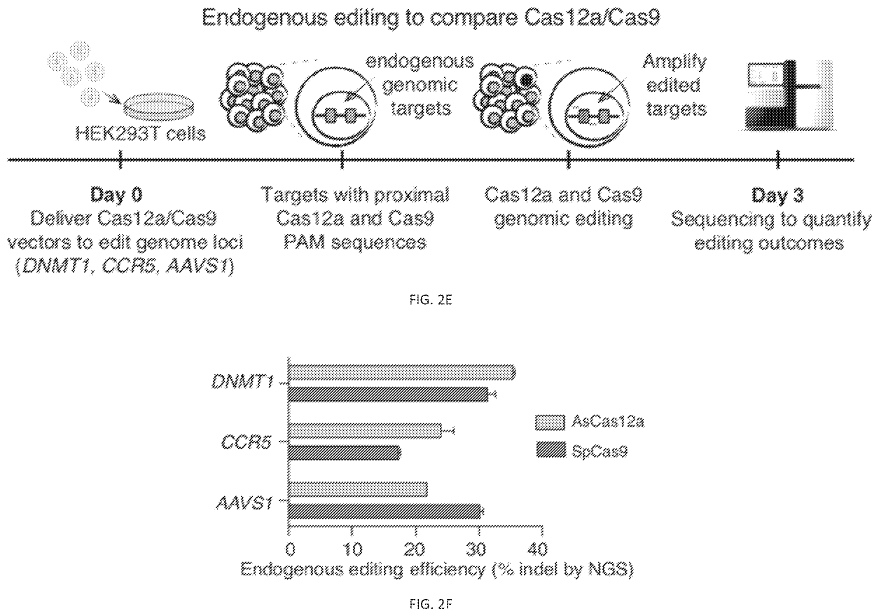

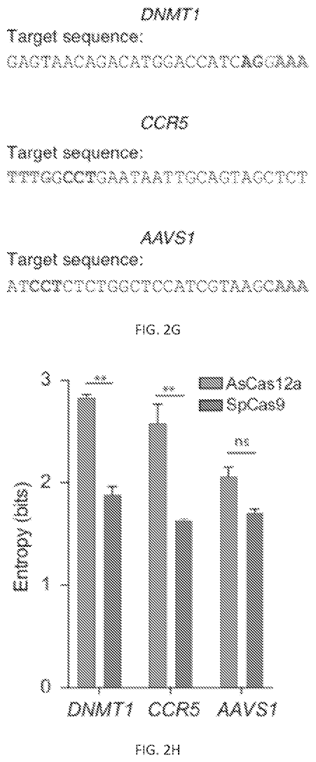

To compare Cas9 and Cas12a editing for their barcoding capacities, cell lines bearing inducible Cas12a or Cas9 expression constructs were generated, where gene-editing was controlled by using a chemical inducer Doxycycline (FIG. 7A). Using these inducible cell lines, the editing entropy of Cas9 and Cas12a was examined as they generate indels, and the resulting entropies were compared across three barcode sequences from a published study (FIGS. 2A-2B). A two-target design was chosen to generate a minimal but functional multi-target barcode. The entropy of editing outcomes in each barcode was evaluated after lentiviral delivery and doxycycline-induced expression of Cas12a (AsCas12a and enhanced enAsCas12a) or Cas9 (SpCas9) (FIGS. 2A-2B). Across all tests, Cas12a-based editing consistently led to higher barcode entropy than Cas9 (FIG. 2C). In support, the read frequencies of unique editing outcomes were illustrated, confirming that Cas12a yields a higher number of unique outcomes and their distribution is closer to uniform compared with Cas9 (FIG. 2D).

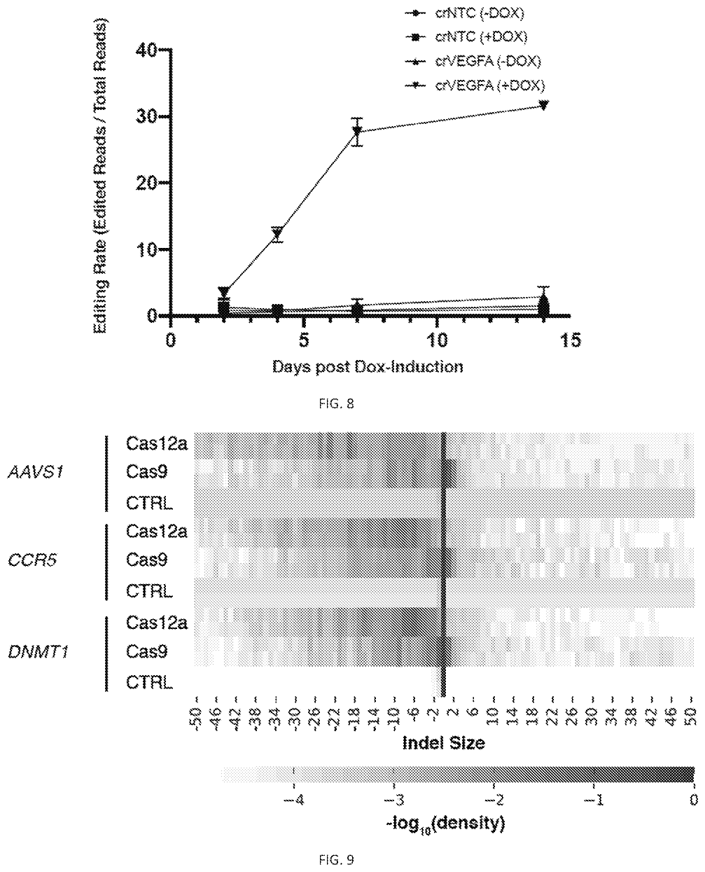

Further, Cas9 and Cas12a were tested and their editing entropies were evaluated within three endogenous genome loci DNMT1, CCR5, AAVS1 (FIG. 2E). The Cas9 and the Cas12a target sequences were selected such that they are proximal (FIGS. 2E and 2G). Endogenous targeting helped to minimize confounding factors and potential variability caused by lentiviral integration. After transient transfection of Cas12a/Cas9 vectors, the genomic targets were sequenced and editing outcomes were quantified (FIG. 9). Across all endogenous loci, Cas12a consistently led to higher entropy and more diverse editing outcomes, while Cas9 often led to outcomes that were dominated by the most frequent indels (FIGS. 2H-2I). The entropy advantage of Cas12a was not relevant to differences in editing efficiency (FIG. 2F). Overall, Cas12a barcodes demonstrated the capacity to generate a wide range of mutations during the course of editing and thus higher entropy.

### Example 2

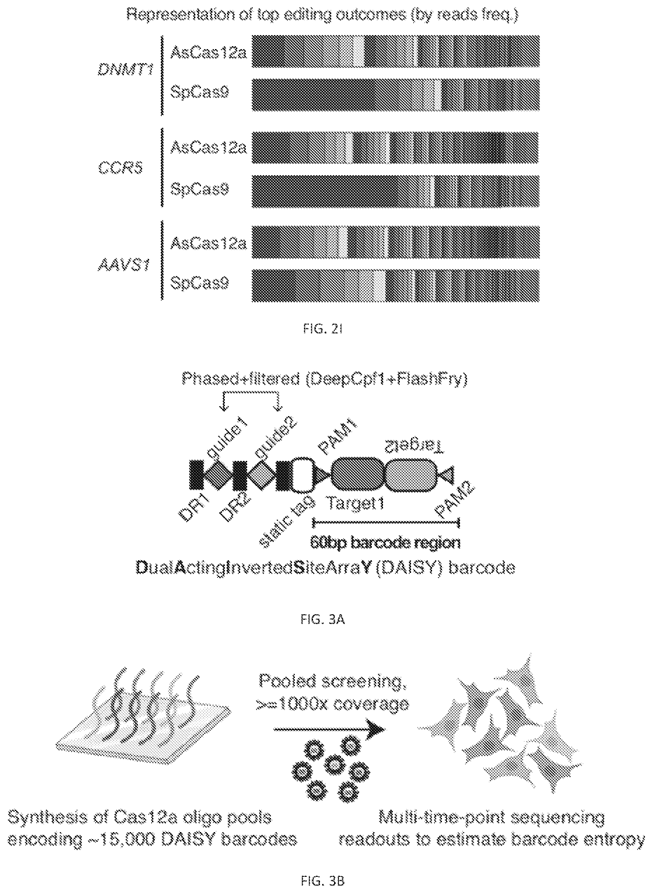

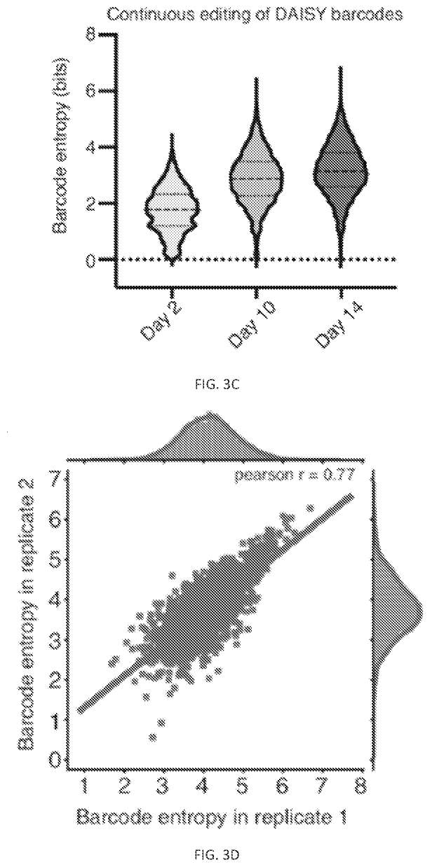

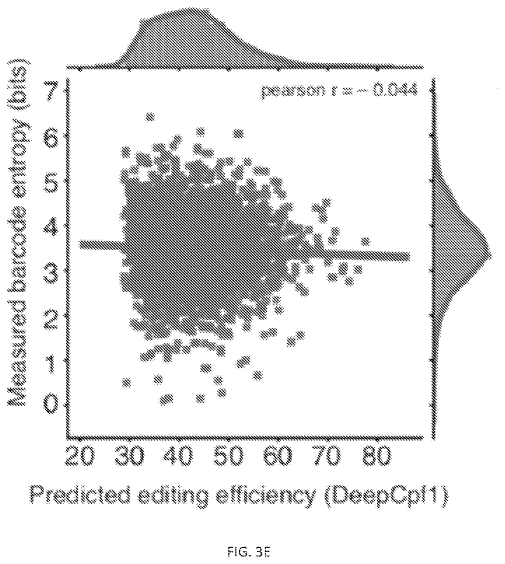

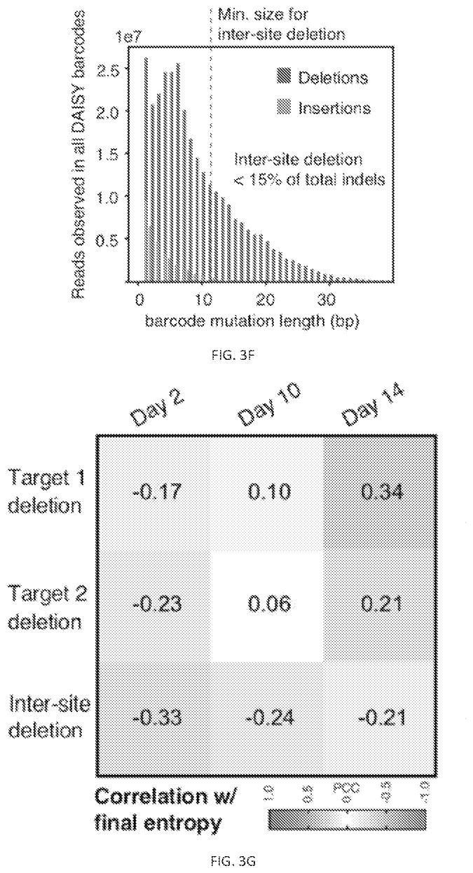

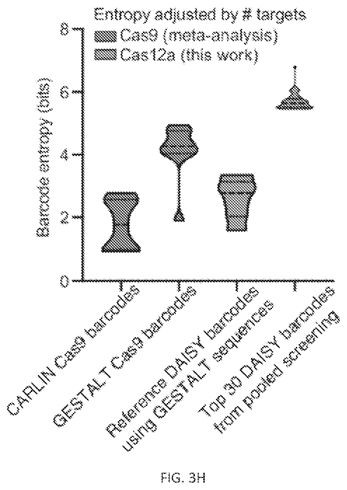

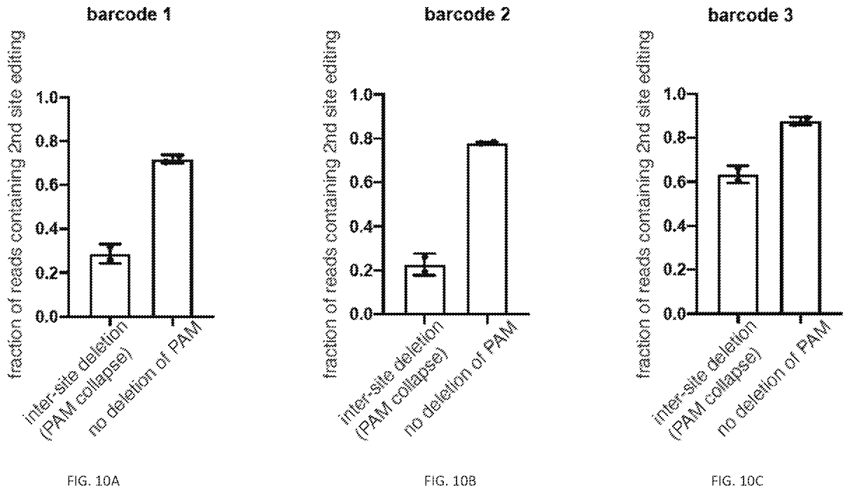

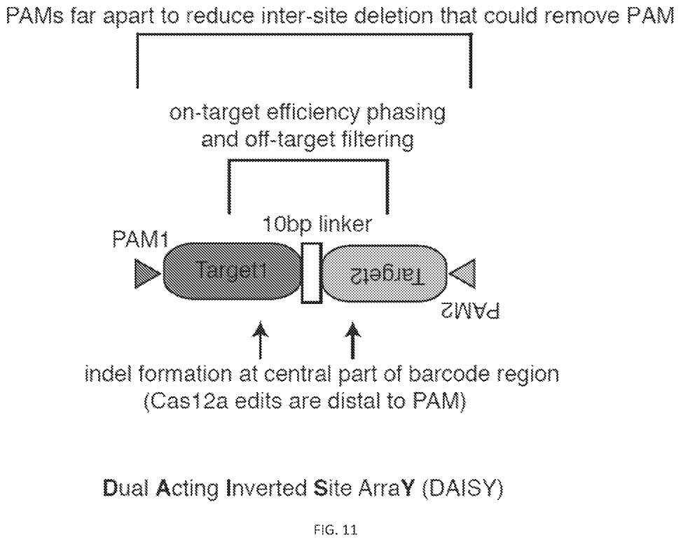

A key limiting factor of Cas9 barcode entropy was inter-site deletions that span two or more target sites within a barcode. When an inter-site deletion happens during the barcoding process, it removes at least one PAM sequence (required for CRISPR editing) and destructs a large region of the barcode. It will also prevent further editing, so that a large number of descendant cells could end up with undistinguishable barcode sequences, thus reducing the overall barcode capacity. Analysis from the initial barcode test also confirmed that levels of inter-site deletion correlate with lower barcode editing efficiencies (up to 3-fold reduction; FIG. 10). To overcome this difficulty, a new Cas12a barcode was developed, referred to as Dual Acting Inverted Site ArraY (DAISY) barcode (FIGS. 3A and 11). A key attribute of DAISY is the inverted two-site design in which two Cas12a PAM sequences are oriented inversely at the ends of barcode region, such that the Cas12a enzyme cleavage sites are centered to minimize the chance of PAM removal due to inter-site deletion.

Importantly, the use of Cas12a in DAISY barcodes allows high-throughput barcode screening. While prior work has leveraged pooled screening to measure CRISPR editing at single sites, there have not been prior attempts to optimize barcode sequence which requires large-scale screening for multisite editing. Leveraging the compactness and multiplex editing of Cas12a, the barcode capacity of a large number of two-target sequences to be used as initial DAISY barcodes was evaluated (FIG. 12). Oligos containing five elements: 1) an evolvable DAISY barcode sequence with two target sites, 2) an array of two crRNAs containing two guide sequences to edit the target sites, 3) termination signal for crRNA expression, 4) a static sequence tag to uniquely identify each initial DAISY barcode sequence, and 5) flanking sequences for cloning (omitted in schematics) were synthesized (FIG. 3A). To measure the entropy of many DAISY barcodes, 5000 random target sequences were generated, all combinations of two target sites were pair-wisely assembled, and the sequences were filtered to keep those with phased efficiency and low off-target scores (FIG. 12, Methods). A lentiviral library was constructed using 14,358 oligos encoding all the Cas12a DAISY barcodes that passed filtering and they were delivered into inducible Cas12a cell lines (FIG. 3B). After initiation of Cas12a editing, the evolving DAISY barcodes were sequenced across multiple time points and aligned to the original barcode sequence to identify the editing outcomes and entropy (FIG. 3B, Methods).

### Example 3

The editing entropy was measured for each sequence in the initial DAISY barcode library, and over the course of the experiment, the median barcode entropy increased monotonically from ˜2 bits at Day 2 to ˜3.5 bits at Day 14 (FIG. 3C). The reproducibility of barcode entropy was confirmed across the two biological replicates (FIG. 3D). Also, high variability in the resulting barcode entropies was observed (FIG. 3D), consistent with initial barcode sequences influencing the barcode capacity. Notably, the barcode entropy of DAISY did not correlate with the DeepCpf1 prediction of editing efficiency (FIG. 3E). This indicated that the complex barcode editing process over two adjacent target sites (a unique feature of the DAISY library) cannot be readily predicted using an existing model based on single-site Cas12a editing data. Further, the majority of observed indels were less than 10 nucleotides in length, providing evidence that DAISY reduces inter-site deletions (given the 10bp linker between target sites, any inter-site deletion would be expected to be at least 10 nucleotides in length) (FIG. 3F). Overall, more than 85% of the barcode sequences evolve without any inter-site deletion.

### Example 4

How the temporal dynamics of barcode editing influences the final barcode capacity was analyzed. The pairwise correlation between the three major types of deletions was calculated and the barcode entropy was measured, across all barcode sequences across time (FIG. 3G). Several trends were apparent from this analysis. First, early deletion events of all types (on Day 2) reduced the chance of further editing and correlated negatively with the final barcode entropy (FIG. 3G, Day2 column). Second, there was a strong negative correlation across all time points between the inter-site deletion and barcode entropy (FIG. 3G, bottom row). In particular, early inter-site deletions at Day 2 had the strongest negative correlation. Third, a positive correlation between single-site editing at later time points (Day10,14) and barcode entropy was observed, which was notable on Day 10 and significantly stronger on Day 14 (FIG. 3G, top two rows). Together, these observations show that preventing inter-site deletion and continuous editing were important to high-capacity Cas12a barcoding.

### Example 5

While the basic DAISY system uses a small two-target-site design, many published CRISPR barcode systems use more than two target sites. Note that longer barcodes are expected to have higher capacity, as multiple target sites could evolve independently and generate more unique outcomes (states). To compare DAISY barcodes with published Cas9 barcodes, two approaches were used for benchmarking in comparable terms. First, for experimental benchmarks, those barcode sequences used in the published Cas9 barcodes from the GESTALT method were included to construct target sites for Cas12a, and as references in the DAISY library. Second, for a meta-analysis, data from two Cas9 barcode studies was analyzed and barcode entropies were calculated by pairing two targets from these Cas9 barcodes. In both experimental and meta-analysis comparisons, the top 30 DAISY barcodes from the initial screen outperformed the Cas9 barcode references (FIG. 3H). Notably, the top DAISY barcode reached an entropy value of ˜6 bits, while the top designs from benchmarks had entropies of ˜4 bits. The 2-bits increase in entropy hints that the total number of unique states could increase by up to 4-fold, with the barcode size fixed.

### Example 6

Evidence from the initial DAISY library screening implied that choice of the initial barcode sequence significantly influenced the final barcode entropy. Thus, optimal choices of the barcode sequence should maximize the capacity for lineage tracking. Exhaustively testing all possible barcode sequences for the CRISPR-based system, even for a most compact 20-bp target, requires screening ˜420 sequences, or ˜1 trillion possibilities. This far exceeds typical experimental throughput. To address this challenge, machine learning (ML) modeling based on the high-throughput DAISY screen Dataset was leveraged. The predictive power of ML-guided search processes was harnessed to design an iterative experiment-computation workflow, termed CRISPR Learning and Optimization via Variants Exploration with Regression (CLOVER) (FIG. 4A). The aim of CLOVER was to use the data from DAISY barcode screening to build an ML model that could predict in silico the entropy of untested barcode sequences, and then select the most promising candidates for focused experimental tests to identify the top barcode designs (FIG. 4A, Methods).

### Example 7

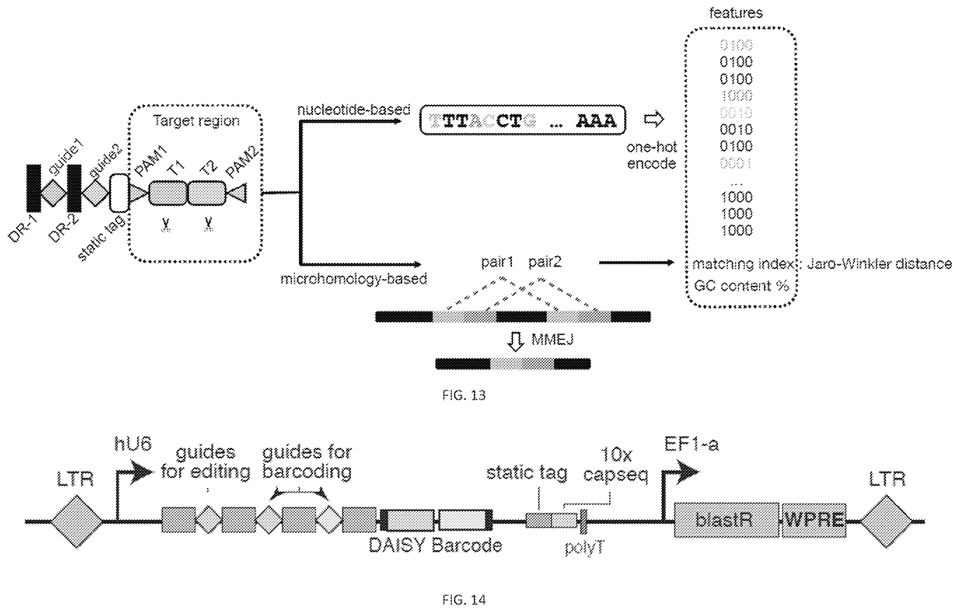

The CLOVER pipeline consists of three modules: feature engineering, entropy prediction, and path-regularized online learning (FIG. 13, Methods). The first module is to engineer a library of features for predictive ML. Inspired by existing machine learning models for single-target CRISPR editing, a collection of features for the DAISY barcodes was constructed, which are based on: (1) one-hot-encoding of nucleotides; (2) GC content information; and (3) a Jaro-Winkler-based distance feature that encoded the process of microhomology-mediated end joining (MMEJ). The Jaro-Winkler gives more weight to the common prefix of two sequences that flank the predicted cut sites. Therefore, it appropriately weighs the increased prevalence of MMEJ-driven editing outcome events as a function of the microhomology tracts distance to the predicted cut site (FIG. 13). The second module is a machine learning-based predicted model to predict a sequence's entropy. A ridge regression model was trained to test the predictive power of the feature space and found that the model was highly predictive of barcode entropy, with a testing r of 0.83 (FIG. 4B, Methods). The third module enables adaptive search and dynamic exploration of the design space via in silico mutagenesis (FIG. 4A, additional details in Methods). A path regularized online learning method was developed using a bandit optimization formulation: at each round of optimization, a learning agent chooses an arm, a combination of designs to experiment on, and receives a stochastic reward. The difference between this reward and the maximal reward at this round, assuming it exists, is defined as instantaneous regret. In the context of the DAISY barcode optimization, the rewards were defined as the average barcode entropy of the identified barcodes and the instantaneous regret is the difference between this value and its maximal-possible value in the pool of sequences. Minimizing the instantaneous regret is equivalent to maximizing the barcode entropy of sequences chosen for new experiments. An upper-confidence bandit learning approach was chosen for recommending new designs utilizing a probabilistic surrogate model. The approach would recommend random new design sequences with probability proportional to a “potential” score, where the score is a combination of the design's predicted entropy and the prediction's level of uncertainty. This would encourage exploring new designs that are highly dissimilar to tested design sequences, which enables fast exploration of large sequence space and fast convergence to the optimal solution.

### Example 8

Based on the first DAISY barcode screen, the CLOVER pipeline was employed to generate a new set of 2000 optimized barcode sequences to synthesize and test in a second pooled screen (Methods). A lentiviral library with new optimized barcodes (DAISY 2nd screen) was generated as well as a set of controls from the original screen (DAISY 1st screen) to serve as internal benchmarks (FIG. 4C). The ML-optimized DAISY barcodes significantly increased the average barcode entropy compared with the 1st screen, with top-performing barcodes achieving an entropy increase of ˜3 bits, which suggested an 8-fold increase of the number of unique lineages that the barcode is capable of tracking (FIG. 4C). In addition, the 2nd screen DAISY barcode sequences were tested in both human melanoma and lung adenocarcinoma cell lines expressing inducible Cas12a. The results demonstrated that the barcode entropy was comparable between the two cell lines, with top barcodes having consistent rankings, showing that optimized DAISY barcode sequences can generalize across cellular contexts (FIG. 4D).

### Example 9

The tunability of two top-performing DAISY barcodes, bc859 and be1095, was tested using the inducible Cas12a cell line (FIG. 4E). Similar to a recently described one-phase exponential decay model, the entropy change rate of these top DAISY barcodes was derived based on longitudinal measurements (FIGS. 4E-4G). By varying the doxycycline concentration, the entropy change rate of the barcodes could be tuned to range from ˜0.25 bits/day (low dosage, slower barcode evolution) to ˜0.5 bits/day (high dosage, faster barcode evolution). This tunability finds use in applications in which the rate of barcode evolution needs to match the biological processes under investigation.

Taken together, these results demonstrated that the optimized Cas12a DAISY barcoding system is compact, high-capacity, and tunable, supporting its potential application in a wide range of biological studies. After adjusting for the number of target sites, the top optimized DAISY barcodes achieved ˜9 bits of entropy, compared with ˜4 bits for Cas9 benchmarks (FIGS. 4D and 4F). Such a 5-bits entropy improvement suggests that the number of unique barcode states could increase by 25-fold. Thus, an optimized 60-bp DAISY barcode can track similar numbers of lineages as a kilobase-scale Cas9 barcode.

### Example 10

To demonstrate the utility of optimized DAISY barcodes, a melanoma cell line model was employed for proof-of-concept single-cell lineage tracking. A top barcode sequence (be 1095) was cloned into a lentiviral vector that would allow edited barcodes to be transcribed and captured by standard single-cell RNA-seq (FIG. 5A). The single-cell DAISY barcode (scDAISY-seq) vector contained a single cassette in which the crRNAs targeting the DAISY barcode was followed by the target sequences, with a static tag to label the founding clonality of cells (FIG. 14). Melanoma cells were transduced with doxycycline-inducible Cas12a and scDAISY-seq vectors. Following selection to enrich for transduced cells, the cells were bottlenecked to have approximately 5 parental cells. Cas12a was induced to initiate editing of the DAISY barcodes, and cells were harvested for single-cell RNA-seq after ˜14 days (FIG. 5A).

### Example 11

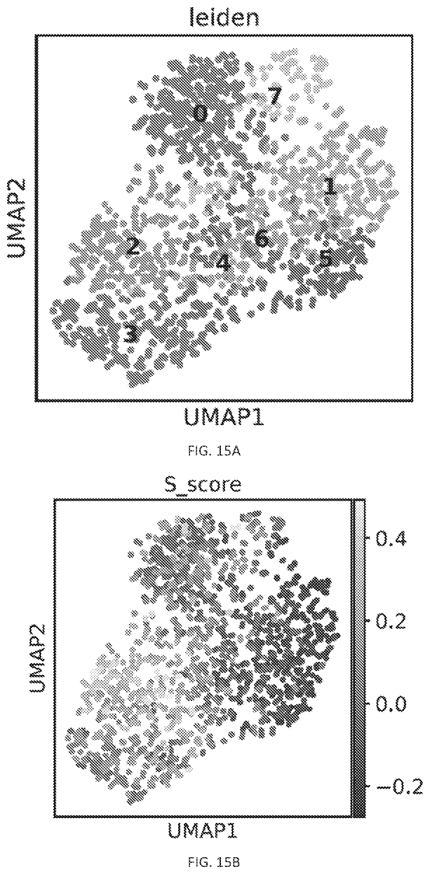

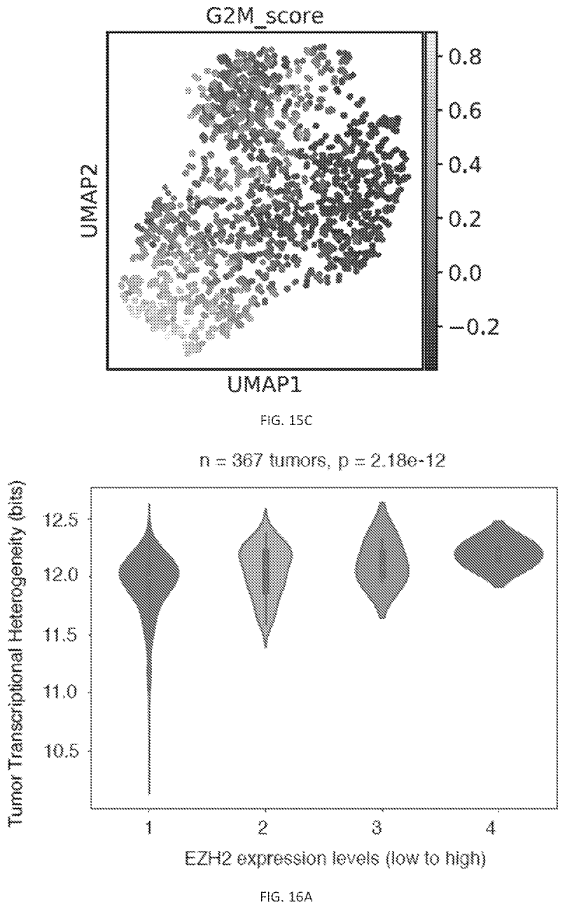

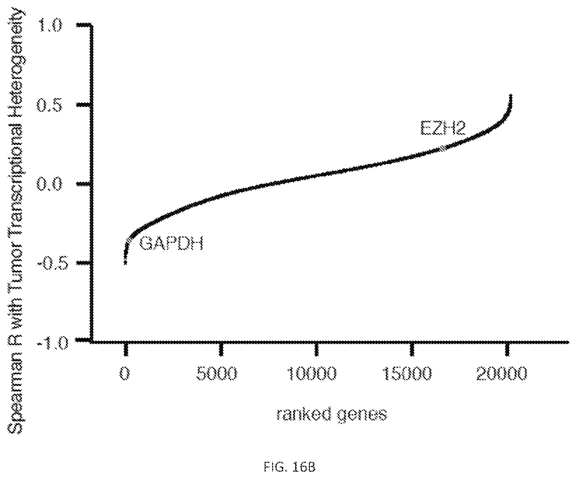

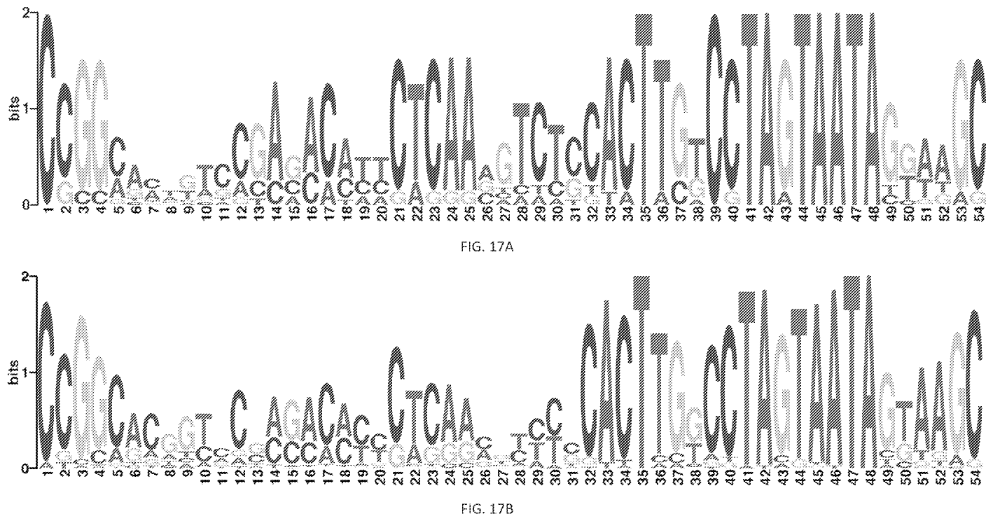

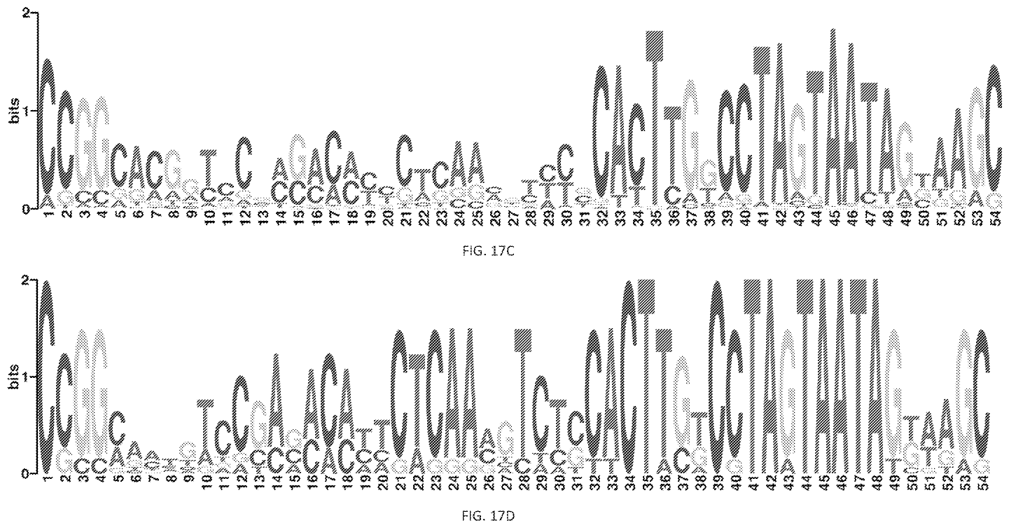

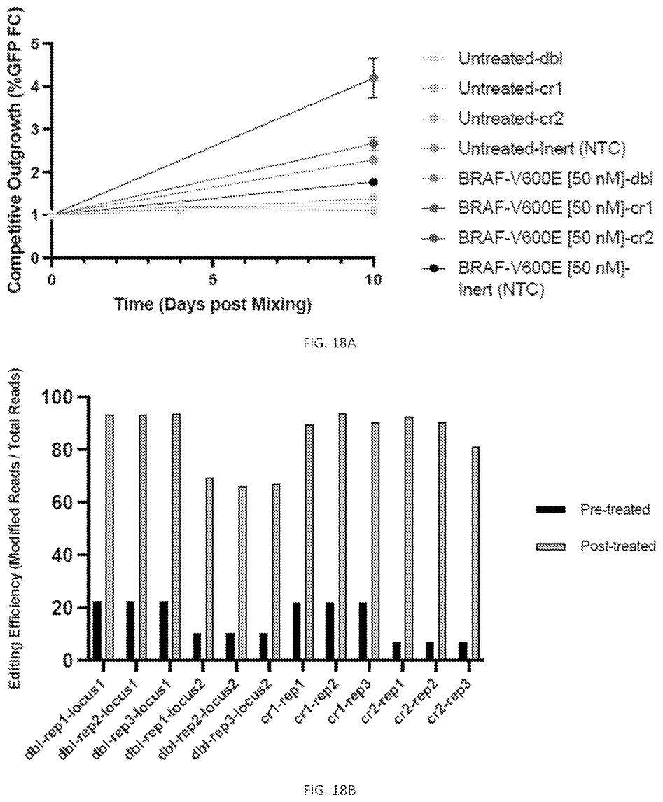

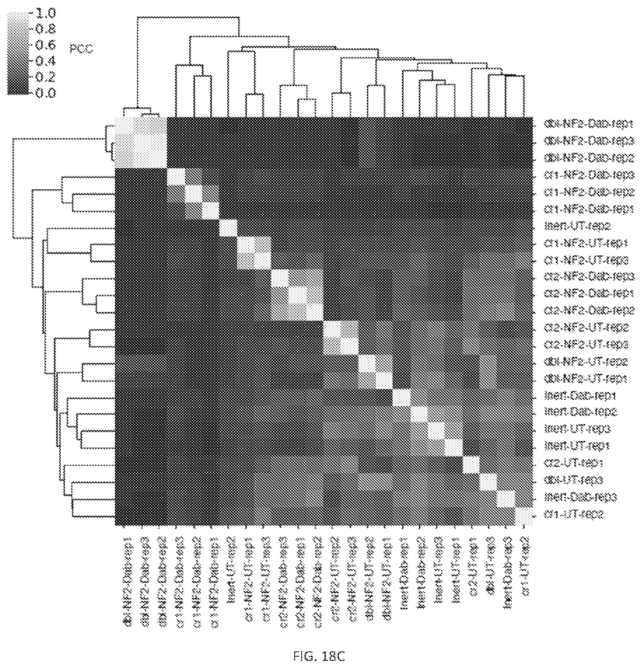

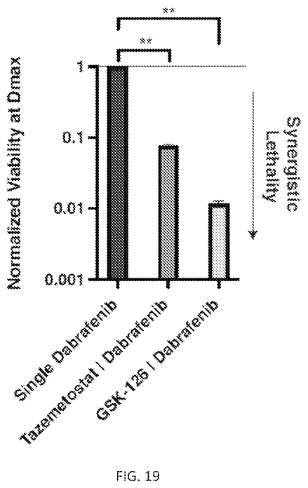

From the single-cell RNA-seq data, sequencing reads corresponding to the DAISY barcodes in ˜2000 cells were recovered, or ˜70% of all cells that passed initial filtering to remove those with poor sequencing quality (FIGS. 5A and 15, Methods). These single-cell DAISY barcode reads harbored a total of 1512 unique editing outcomes, with deletions more prevalent than insertions, as expected from Cas12a editing (FIG. 5B). The bimodal distribution of the editing events within the barcode region (FIG. 5B) demonstrated that most indels were within each of the target sites (T1, T2). This was consistent with the prior observation in bulk that DAISY barcodes are less susceptible to inter-site deletions. The optimized 60-bp DAISY barcode reached an entropy of 9.63 bits, also consistent with the bulk measurements. The largest and dominant clonal population (Clone 1, or C1) defined by the static tag was examined, which contained 1129 cells, with 679 unique edited barcodes that are derived from one initial barcode sequence (FIGS. 5C-5D). In this clone, the majority of edited barcodes did not incur any inter-site deletion (FIG. 5C). Further, 60% of the edited barcode sequences uniquely labeled one descendant cell per sequence within this largest clone (FIG. 5D). These unique editing outcomes from C1 had no observed overlap with barcode sequences from the second largest clone C2 (FIG. 5E). This meant that, despite its small size, the evolvable DAISY barcode had tracked a significant portion of cell lineages at single-cell resolution. Such lineage tracking capacity of the optimized 60-bp DAISY barcode is comparable to several Cas9 barcodes, which are often longer, though using multiple copies of the same barcode via transposition or repeated lentiviral transduction could boost the barcode capacity (FIG. 5F). Next, the lineage tree of this clone was reconstructed based on edited barcodes, by using the CASSIOPEIA tool previously developed for Cas9 barcodes. In this step, cells that had low-quality transcriptomic profiles were filtered out to facilitate downstream gene expression analysis (FIG. 5G, Methods).

### Example 12

The lineage history recovered from the DAISY barcode together was used with single-cell transcriptomic profiles, to investigate the inheritance of gene expression that is known to associate with distinct cell behaviors. When such lineage and transcriptomic information are available, one can characterize the heritability of gene expressions patterns (FIG. 5H). Using the scDAISY-seq data, the uniformity of gene expression within barcode-defined lineage groups was calculated and compared with a baseline averaged from randomized non-lineage groups (FIG. 5I). The strength of heritable gene expression, or transcriptional memory, was measured through computing a memory index for each gene (FIGS. 5I-5J, Methods). Then, by ranking genes according to the computed memory indices, a subset of high-memory genes exhibiting highly heritable expression patterns across cells was identified (FIG. 5J).

Top gene sets enriched within high-memory genes identified via DAISY barcoding were examined, identifying that neuronal and chromatin-related pathways are top-ranking (FIG. 5K). The notable associations with neuronal genes in this melanoma cell experiment is intriguing as human melanomas arise from melanocytes that originate from neural crest cells, which can differentiate into neurons. This is potentially significant as a rare neural crest stem cell state has been associated with therapeutic resistance in melanoma. In addition, the chromatin gene enrichment suggested that a feedback mechanism may be involved in maintaining heritable gene expression through epigenetic regulation. To assess this possibility, meta-analysis was conducted using ENCODE data and enriched proteins that bound proximally to these high memory genes were identified (FIG. 5L). Intriguingly, two top proteins, EZH2 and SUZ12, are members of the polycomb repressive complex 2 (PRC2) and play key roles in epigenetic regulation of gene expression (FIG. 5L). In support of this putative epigenetic regulation of transcriptional memory, strong enrichment of EZH2 peaked at the transcriptional start sites (TSSs) of identified high-memory genes, in contrast to control genes with similar expression levels, using ChIP-seq data from melanoma cells (FIG. 5M). Taken together, this demonstration shows that DAISY barcoding coupled with single-cell RNA-seq tracks cell lineage history with simultaneous transcriptomic profiling, providing an approach to investigate complex cell dynamics and their epigenetic regulation.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A system comprising:
a Cas12a protein or a vector encoding thereof;
a polynucleotide barcode flanked by two PAM sequences of inverse orientation, or a vector encoding thereof, wherein the polynucleotide barcode comprises a first target nucleic acid sequence and a second target nucleic acid sequence; and
a pair of guide RNAs (gRNAs) configured to hybridize to the two PAM sequences.

2. The system of claim 1, wherein the system comprises two or more polynucleotide barcodes.

3. The system of claim 1 or 2, wherein the polynucleotide barcode is on the vector encoding the pair of gRNAs.

4. The system of any of claims 1-3, wherein the vector encoding the Cas12a protein is the same vector as the vector encoding the pair of gRNAs.

5. The system of any of claims 1-4, wherein the polynucleotide barcode is on same vector as the pair of gRNAs and the Cas12a protein.

6. The system of any of claims 1-5, wherein the polynucleotide barcode further comprises a linker between the first target nucleic acid sequence and the second target nucleic acid sequence.

7. The system of claim 6, wherein the linker comprises 1-20 nucleotides.

8. The system of claim 6 or claim 7, wherein the linker comprises 10 nucleotides.

9. The system of any of claims 1-8, wherein the polynucleotide barcode comprises less than 200 nucleotides.

10. The system of any of claims 1-9, wherein the polynucleotide barcode comprises less than 150 nucleotides.

11. The system of any of claims 1-10, wherein the polynucleotide barcode comprises 50-60 nucleotides.

12. The system of any of claims 1-11, wherein the polynucleotide barcode comprises 54 nucleotides.

13. The system of any of claims 1-12, wherein the polynucleotide barcode sequence is configured to promote insertions and deletions over time.

14. The system of any of claims 1-13, wherein the polynucleotide barcode comprises GC directly upstream of PAM sequence at the 3′ end of the polynucleotide barcode.

15. The system of any of claims 1-14, wherein the polynucleotide barcode comprises a cytidine at position 39.

16. The system of claim 15, wherein the polynucleotide barcode comprises a guanosine at position 40.

17. The system of any of claims 1-16, wherein the polynucleotide barcode comprises an adenosine at positions 45 and 46.

18. The system of any of claims 1-17, wherein the polynucleotide barcode comprises an adenosine at position 31 and a guanosine at position 32.

19. The system of any of claims 1-18, wherein the polynucleotide barcode does not comprise a thymidine at position 54, position 49, or both.

20. The system of any of claims 1-19, wherein the polynucleotide barcode does not comprise a guanosine at position 50 and a cytidine at position 51.

21. The system of any of claims 1-20, wherein the polynucleotide barcode comprises CACTTG (SEQ ID NO: 1054) at positions 32-37.

22. The system of any of claims 1-21, wherein the polynucleotide barcode comprises CCTAGTAATAG (SEQ ID NO: 1055) at positions 39-49.

23. The system of any of claims 1-22, wherein the polynucleotide barcode comprises CCGG (SEQ ID NO: 1056) directly downstream of PAM sequence at the 5′ end of the polynucleotide barcode.

24. The system of any of claims 1-23, wherein the polynucleotide barcode comprises a sequence with at least 70% similarity to sequences selected from the group consisting of SEQ ID NOs: 1-1053.

25. The system of any of claims 1-24, wherein the polynucleotide barcode sequence is selected from the group consisting of SEQ ID NOs: 1-10.

26. The system of any of claims 1-25, wherein the pair of gRNAs are within a crRNA array.

27. The system of any of claims 1-26, wherein one or each of the pair of gRNAs comprise a guide sequence of less than 25 nucleotides.

28. The system of any of claims 1-27, further comprising at least one gRNA configured to hybridize to a recipient nucleic acid.

29. The system of claim 28, wherein the recipient nucleic acid is a nucleic acid endogenous to a target cell.

30. The system of claim 28 or 29, wherein the recipient nucleic acid is a gene within a target cell.

31. The system of any of claims 28-30, wherein the at least one gRNA configured to hybridize to a recipient nucleic acid is on the vector encoding the pair of gRNAs.

32. The system of any of claims 28-31, wherein the at least one gRNA is within the crRNA array comprising the pair of gRNAs.

33. The system of any of claims 1-32, wherein at least one or all of the gRNAs are non-naturally occurring gRNAs.

34. The system of any of claims 1-33, wherein the vector encoding Cas12a comprises an inducible promoter for Cas12a expression.

35. The system of any of claims 1-34, wherein the system further comprises a recipient nucleic acid.

36. The system of any of claims 1-35, further comprising a sequence tag configured to remain static over time.

37. The system of any of claims 1-36, further comprising a gene editing system.

38. The system of claim 37, wherein the gene editing system comprises a CRISPR/Cas gene editing system.

39. The system of claim 37 or 38, further comprising one or more gene editing gRNAs.

40. The system of claim 39, wherein the one or more gene editing gRNAs are provided in a crRNA array with the pair of guide RNAs.

41. The system of any of claims 1-40, wherein the system is a cell-free system.

42. A cell comprising the system of any one of claims 1-40.

43. The cell of claim 42, wherein the cell is a eukaryotic cell.

44. The cell of claim 42 or 43, wherein the cell is in vitro.

45. The cell of any of claims 42-44, wherein the cell is a cancer cell.

46. A system comprising:
a CRISPR associated (Cas) endonuclease or a vector encoding thereof;
a polynucleotide barcode comprising less than 100 nucleotides flanked by two PAM sequences; and
a pair of guide RNAs (gRNAs) configured to hybridize to the two PAM sequences.

47. The system of claim 46, wherein the system comprises two or more polynucleotide barcodes.

48. The system of claim 46 or 47, wherein the polynucleotide barcode is on the vector encoding the pair of gRNAs.

49. The system of any of claims 46-48, wherein the vector encoding the Cas endonuclease is the same vector as the vector encoding the pair of gRNAs.

50. The system of any of claims 46-49, wherein the polynucleotide barcode is on same vector as the pair of gRNAs and the Cas endonuclease.

51. The system of any of claims 46-50, wherein the polynucleotide barcode further comprises first target nucleic acid sequence and the second target nucleic acid sequence.

52. The system of any of claims 46-51, wherein the PAM sequences are of inverse orientation.

53. The system of any of claims 46-52, wherein the polynucleotide barcode comprises 50-60 nucleotides.

54. The system of any of claims 46-53, wherein the polynucleotide barcode comprises 54 nucleotides.

55. The system of any of claims 46-54, wherein the polynucleotide barcode sequence is configured to promote insertions and deletions over time.

56. The system of any of claims 46-55, wherein the polynucleotide barcode comprises GC directly upstream of PAM sequence at the 3′ end of the polynucleotide barcode.

57. The system of any of claims 46-56, wherein the polynucleotide barcode comprises a cytidine at position 39.

58. The system of claim 57, wherein the polynucleotide barcode comprises a guanosine at position 40.

59. The system of any of claims 46-58, wherein the polynucleotide barcode comprises an adenosine at positions 45 and 46.

60. The system of any of claims 46-59, wherein the polynucleotide barcode comprises an adenosine at position 31 and a guanosine at position 32.

61. The system of any of claims 46-60, wherein the polynucleotide barcode does not comprise a thymidine at position 54, position 49, or both.

62. The system of any of claims 46-61, wherein the polynucleotide barcode does not comprise a guanosine at position 50 and a cytidine at position 51.

63. The system of any of claims 46-62, wherein the polynucleotide barcode comprises CACTTG (SEQ ID NO: 1054) at positions 32-37.

64. The system of any of claims 46-63, wherein the polynucleotide barcode comprises CCTAGTAATAG (SEQ ID NO: 1055) at positions 39-49.

65. The system of any of claims 46-64, wherein the polynucleotide barcode comprises CCGG (SEQ ID NO: 1056) directly downstream of PAM sequence at the 5′ end of the polynucleotide barcode.

66. The system of any of claims 46-65, wherein the polynucleotide barcode comprises a sequence with at least 70% similarity to sequences selected from the group consisting of SEQ ID NOs: 1-1053.

67. The system of any of claims 46-66, wherein the polynucleotide barcode sequence is selected from the group consisting of SEQ ID NOs: 1-10.

68. The system of any of claims 46-67, wherein the pair of gRNAs are within a crRNA array.

69. The system of any of claims 46-68, wherein one or each of the pair of gRNAs comprise a guide sequence of less than 25 nucleotides.

70. The system of any of claims 46-69, further comprising at least one gRNA configured to hybridize to a recipient nucleic acid.

71. The system of claim 70, wherein the recipient nucleic acid is a nucleic acid endogenous to a target cell.

72. The system of claim 70 or 71, wherein the recipient nucleic acid is a gene within a target cell.

73. The system of any of claims 70-72, wherein the at least one gRNA configured to hybridize to a recipient nucleic acid is on the vector encoding the pair of gRNAs.

74. The system of any of claims 70-73, wherein the at least one gRNA is within the crRNA array comprising the pair of gRNAs.

75. The system of any of claims 46-74, wherein at least one or all of the gRNAs are non-naturally occurring gRNAs.

76. The system of any of claims 46-75, wherein the vector encoding Cas12a comprises an inducible promoter for Cas12a expression.

77. The system of any of claims 46-76, wherein the system further comprises a recipient nucleic acid.

78. The system of any of claims 46-77, further comprising a sequence tag configured to remain static over time.

79. The system of any of claims 46-78, further comprising a gene editing system.

80. The system of claim 79, wherein the gene editing system comprises a CRISPR/Cas gene editing system.

81. The system of claim 79 or 80, further comprising one or more gene editing gRNAs.

82. The system of claim 81, wherein the one or more gene editing gRNAs are provided in a crRNA array with the pair of guide RNAs.

83. The system of any of claims 46-82, wherein the system is a cell-free system.

84. A population of cells comprising the system of any one of claims 46-82.

85. The population of cells of claim 84, wherein each of the cells comprises a distinct version of the barcode representing a particular cell generation.

86. The population of cells of claim 85, wherein the population of cells represents up to 1000 cell generations.

87. The population of cells of claim 85 or 86, wherein the population of cells represents about 700 cell generations.

88. The population of cells of any of claims 84-87, wherein the population comprises eukaryotic cells.

89. The population of cells of any of claims 84-88, wherein the population of cells is in vitro.

90. The population of cells of any of claims 84-89, wherein the population of cells comprises cancer cells.

91. A nucleic acid comprising a polynucleotide barcode flanked by a pair of PAM sequences of inverse orientation, wherein the polynucleotide barcode comprises a first target nucleic acid sequence and a second target nucleic acid sequence.

92. The nucleic acid of claim 91, wherein the polynucleotide barcode further comprises a linker between the first target nucleic acid sequence and the second target nucleic acid sequence.

93. The nucleic acid of claim 92, wherein the linker comprises 1-20 nucleotides.

94. The nucleic acid of claim 92 or claim 93, wherein the linker comprises 10 nucleotides.

95. The nucleic acid of any of claims 91-94, wherein the polynucleotide barcode comprises less than 200 nucleotides.

96. The nucleic acid of any of claims 91-95, wherein the polynucleotide barcode comprises less than 150 nucleotides.

97. The nucleic acid of any of claims 91-96, wherein the polynucleotide barcode comprises less than 100 nucleotides.

98. The nucleic acid of any of claims 91-97, wherein the polynucleotide barcode comprises 50-60 nucleotides.

99. The nucleic acid of any of claims 91-98, wherein the polynucleotide barcode sequence is configured to promote insertions and deletions over time.

100. The nucleic acid of any of claims 91-99, wherein the polynucleotide barcode comprises GC at the 3′ end of the polynucleotide barcode.

101. The nucleic acid of any of claims 91-100, wherein the polynucleotide barcode comprises a cytidine at position 39.

102. The nucleic acid of claim 101, wherein the polynucleotide barcode comprises a guanosine at position 40.

103. The nucleic acid of any of claims 91-102, wherein the polynucleotide barcode comprises an adenosine at positions 45 and 46.

104. The nucleic acid of any of claims 91-103, wherein the polynucleotide barcode comprises an adenosine at position 31 and a guanosine at position 32.

105. The nucleic acid of any of claims 91-104, wherein the polynucleotide barcode does not comprise a thymidine at position 54, position 49, or both.

106. The nucleic acid of any of claims 91-105, wherein the polynucleotide barcode does not comprise a guanosine at position 50 and a cytidine at position 51.

107. The nucleic acid of any of claims 91-106, wherein the polynucleotide barcode comprises CACTTG (SEQ ID NO: 1054) at positions 32-37.

108. The nucleic acid of any of claims 91-107, wherein the polynucleotide barcode comprises CCTAGTAATAG (SEQ ID NO: 1055) at positions 39-49.

109. The nucleic acid of any of claims 91-108, wherein the polynucleotide barcode comprises CCGG (SEQ ID NO: 1056) at the 5′ end of the polynucleotide barcode.

110. The nucleic acid of any of claims 91-109, wherein the polynucleotide barcode comprises a sequence with at least 70% similarity to sequences selected from the group consisting of SEQ ID NOs: 1-1053.

111. The nucleic acid of any of claims 91-110, wherein the polynucleotide barcode sequence is selected from the group consisting of SEQ ID NO: 1-10.

112. The nucleic acid of any of claims 91-111, wherein the nucleic acid encodes a pair of gRNAs configured to hybridize to the two PAM sequences flanking the polynucleotide barcode.

113. The nucleic acid of claim 112, wherein one or both of the pair of gRNAs comprises a guide sequence of less than 25 nucleotides.

114. The nucleic acid of any of claims 91-113, wherein the pair of gRNAs are within a crRNA array.

115. The nucleic acid of claim 114, wherein the crRNA array further comprises a termination signal for crRNA expression.

116. The nucleic acid of claim 114 or 115, wherein the crRNA array further comprises at least one gRNA configured to hybridize to a recipient nucleic acid.

117. The nucleic acid of any of claims 91-116, wherein the nucleic acid further comprises a sequence tag configured to remain static over time.

118. A method for introducing a polynucleotide barcode into a cell comprising:
introducing into the cell a system of any of claims 1-40, a system of any of claims 46-82, or the nucleic acid of any of claims 91-117 and a CRISPR associated (Cas) endonuclease or a nucleic acid encoding a Cas endonuclease.

119. The method of claim 118, wherein the Cas endonuclease is a Class 2 Cas endonuclease.

120. The method of claim 118 or 119, wherein the Cas endonuclease is a Type V Cas endonuclease.

121. The method of any of claims 118-120, wherein the Cas endonuclease is selected from Cas9, Cas12a, and Cas14.

122. The method of any of claims 118-121, wherein the polynucleotide barcode integrates into genomic DNA.

123. The method of any of claims 118-122, wherein the polynucleotide barcode is passed to daughter cells.

124. The method of any of claims 118-123, wherein the cell is eukaryotic cell.

125. The method of any of claims 118-124, wherein the cell is in vitro.

126. The method of any of claims 118-125, wherein the cell is ex vivo.

127. The method of any of claims 118-126, wherein the cell is in a subject.

128. A method for cell tracking comprising:
introducing in the cell:
a system of any of claims 1-40,
a system of any of claims 46-82, or
the nucleic acid of any of claims 91-117 and a Cas endonuclease;
isolating cellular nucleic acids at one or more time points;
sequencing the polynucleotide barcode at the one or more time points; and
tracking changes to original sequence of barcode in the cell at each time point.

129. The method of claim 128, wherein the cell is eukaryotic cell.

130. The method of claim 128 or 129, wherein the cell is in vitro.

131. The method of any of claims 128-130, wherein the cell is ex vivo.

132. The method of claim 128 or 129, wherein the cell is in a subject.

133. The method of any of claims 128-132, wherein the one or more time points are over multiple cell generations.

134. The method of any of claims 128-133, further comprising establishing lineage connections or a sequence of changes in barcode sequence between cells from different generations.

135. The method of any of claims 128-134, wherein expression of Cas12a or the Cas endonuclease is controlled by an inducible promoter.

136. The method of claim 135, further comprising adding varying concentrations of an inducing agent to the cells to vary the change rate of the original barcode sequence.

137. The method of claim 136, wherein increasing concentrations of the inducing agent increases the change rate of the original barcode sequence.

138. The method of any of claims 135-137, wherein the cell is a tumor cell, a neuron, or an adipocyte.

139. The method of claim 138, wherein the inducing agent concentration is low.

140. The method of any of claims 135-137, wherein the cell is a cancerous cell or intestinal epithelial cell.

141. The method of claim 140, wherein the inducing agent concentration is high.

142. The method of any of claims 128-141, further comprising determining single-cell transcriptomic profiles.

143. The method of claim 142, further comprising characterizing heritability of gene expression patterns.

144. The method of claim 143, further comprising determining gene products which have heritable expression patterns.

145. The method of any of claims 128-144, further comprising introducing one or more mutations, insertions, or deletions in one or more target genes of interest in the cell.

146. The method of claim 145, further comprising monitoring the effect of the one or more mutations, insertions, or deletions on cell function, cell viability, or effectiveness of a pharmacological treatment.

147. A computer implemented method for designing a polynucleotide barcode sequence configured to promote insertions and deletions over time comprising:
designing a seed barcode sequence based on sequence elements which promote insertions and deletions, sequence elements which suppress insertions and deletions, or both;
iteratively mutating the seed barcode sequence; and
predicting sequence entropy as measure of insertions and deletions accumulated in the polynucleotide barcode sequence over time.

148. The computer implemented method of claim 147, wherein the seed barcode comprises 50-60 nucleotides.

149. The computer implemented method of claim 147 or 148, wherein the sequence elements which promote insertions and deletions are selected from the group consisting of: a GC dinucleotide at the 3′ end of the barcode; a CG dinucleotide starting at position 39; an AA dinucleotide starting at position 45; an AG dinucleotide starting at position 31; CACTTG (SEQ ID NO: 1054) at positions 32-37; CCTAGTAATAG (SEQ ID NO: 1055) at positions 39-49; CCGG (SEQ ID NO: 1056) at the 5′ end of the polynucleotide barcode; or a combination thereof.

150. The computer implemented method of any of claims 147-149, wherein the sequence elements which suppress insertions and deletions are selected from the group consisting of: a thymidine at position 54; a thymidine at position 49; a GC dinucleotide starting at position 50; or a combination thereof.


SEQUENCE LISTING (1106 sequences)

SEQ ID NO: 1 - DNA, 54 bp, artificial sequence
  Synthetic
ccggcacatcgagagacttcctcaaggactcgaattgtcctagtaatacgatgc

SEQ ID NO: 2 - DNA, 54 bp, artificial sequence
  Synthetic
cgcgagacgaacccccacatctcaacatctccacttctcgtagtaataggaagc

SEQ ID NO: 3 - DNA, 54 bp, artificial sequence
  Synthetic
ccggcacattccgagacattctcaacgtctccacttgtcctagtaataggtagc

SEQ ID NO: 4 - DNA, 54 bp, artificial sequence
  Synthetic
ccggcaatgtccgacacaaactcaaagtctctacttgtcctagtaatagtaagc

SEQ ID NO: 5 - DNA, 54 bp, artificial sequence
  Synthetic
ccggaaatgtccgaaacatactcaaagtctccactagacctagtaatagtaggc

SEQ ID NO: 6 - DNA, 54 bp, artificial sequence
  Synthetic
ccggcacattccgagacattctcaaggtctccacttctcctaataatagcggag

SEQ ID NO: 7 - DNA, 54 bp, artificial sequence
  Synthetic
ccgccgtcaggataacccctctcaaagtctgcacttggcctagtaatagtaagc

SEQ ID NO: 8 - DNA, 54 bp, artificial sequence
  Synthetic
ccgggtggctcggagacattcacaaagtcagctcttggcctagtaatatgtagc

SEQ ID NO: 9 - DNA, 54 bp, artificial sequence
  Synthetic
cgggacatgatcccccaaccgagaagcctctcacttgtcctagtaataggttgc

SEQ ID NO: 10 - DNA, 54 bp, artificial sequence
  Synthetic
ccggcactgtccgagaccccctcggcttacgcacttggcctagtaatagtaagc

SEQ ID NO: 11 - DNA, 54 bp, artificial sequence
  Synthetic
ccggagggcctgatgaccctctcagcttacgcactcgtcttagtaataggaagc

SEQ ID NO: 12 - DNA, 54 bp, artificial sequence
  Synthetic
ccggcacgctccgaggcattctcagctctcccacttggcctagtaatagtaagc

SEQ ID NO: 13 - DNA, 54 bp, artificial sequence
  Synthetic
ccggcacggctgatgacccccctcccaatctcacttgtcctagtaatagtaagc

SEQ ID NO: 14 - DNA, 54 bp, artificial sequence
  Synthetic
ccggcacattccgagacattctcaaggtctcgacttgtcctagtaatacgaagc

SEQ ID NO: 15 - DNA, 54 bp, artificial sequence
  Synthetic
cgacctcaatggccgaccagccgccgtgacccacttggcctagtaatagtaagc

SEQ ID NO: 16 - DNA, 54 bp, artificial sequence
  Synthetic
ccggcacggtcttagccgccctcggctctcccacttggcctagtaatagtaagc

SEQ ID NO: 17 - DNA, 54 bp, artificial sequence
  Synthetic
ccggcacggtccgagagagtctcaaaaactgcactaggcctagtaatagttagc

SEQ ID NO: 18 - DNA, 54 bp, artificial sequence
  Synthetic
ccggtacggtgcgctaccccctcggctctcccacttggcctagtaatagtaagc

SEQ ID NO: 19 - DNA, 54 bp, artificial sequence
  Synthetic
ccggcacggcacccccaaccgagagcttacgcacttggcctagtaatagtaagc

SEQ ID NO: 20 - DNA, 54 bp, artificial sequence
  Synthetic
ccgccacggcacccccaaccgagaagcctctcacttgtcctagtaatagtaagc

SEQ ID NO: 21 - DNA, 54 bp, artificial sequence
  Synthetic
ctggagatgaacccccccccctcggcttacgcacttggcctagtaatagtaagc

SEQ ID NO: 22 - DNA, 54 bp, artificial sequence
  Synthetic
ccggcgcggcacccccaaccgagaagcctctcacttgtcctactaatacgaagc

SEQ ID NO: 23 - DNA, 54 bp, artificial sequence
  Synthetic
ccggcacagtccgagaccccctcggctctcccacttggcctagtaatagtaagc

SEQ ID NO: 24 - DNA, 54 bp, artificial sequence
  Synthetic
ctggcgcggtccgagacattctcaaagtcaacacttggcctagtaatagtaagc

SEQ ID NO: 25 - DNA, 54 bp, artificial sequence
  Synthetic
cgggagatgacccccccatgctcacaggctccacttgtgctagtaatagtctac

SEQ ID NO: 26 - DNA, 54 bp, artificial sequence
  Synthetic
ccggcacggtcccagacccccacggctctcccacttggcctagtaatagtaagc

SEQ ID NO: 27 - DNA, 54 bp, artificial sequence
  Synthetic
ccggcacggtccaagacatgctcaaagtctgcacttggcctagtaatagtaagc

SEQ ID NO: 28 - DNA, 54 bp, artificial sequence
  Synthetic
ccggcacggcacccccaaccgagaactctcccacttggcctagtaatagtaagc

SEQ ID NO: 29 - DNA, 54 bp, artificial sequence
  Synthetic
ccggcacgctccaagaccccctcggcacccccacttggcctagtaatagtaagc

SEQ ID NO: 30 - DNA, 54 bp, artificial sequence
  Synthetic
ccggcacggtccgagaccctctcaaagtctgcacttggcctagtaatagtaagc

SEQ ID NO: 31 - DNA, 54 bp, artificial sequence
  Synthetic
acgggacggtccgagaccccctcggctctcccacttggcctagtaatagtaagc

SEQ ID NO: 32 - DNA, 54 bp, artificial sequence
  Synthetic
ccgccgtcatgatagaccccctcggctctcccacttggcctagtaatagtaagc

SEQ ID NO: 33 - DNA, 54 bp, artificial sequence
  Synthetic
ccggcagggcaaccccaaccgagaagcctcacacttgtcctagtaatagtacgc

SEQ ID NO: 34 - DNA, 54 bp, artificial sequence
  Synthetic
ccggctcggtccgagacattctcaaagtctgcacttggcctagtaatagtaagc

SEQ ID NO: 35 - DNA, 54 bp, artificial sequence
  Synthetic
cccccccgtttcgagaccccctcggctctcccacttggcctagtaatagtaagc

SEQ ID NO: 36 - DNA, 54 bp, artificial sequence
  Synthetic
ccggcacggcacccctccccctaggccaacggacttcccctagtgataagaagc

SEQ ID NO: 37 - DNA, 54 bp, artificial sequence
  Synthetic
ccggcacggcatccccaacggagaagcctcgcacttgtcctacgattatgaagc

SEQ ID NO: 38 - DNA, 54 bp, artificial sequence
  Synthetic
ccggcacggcacccccaaccgagaagcctctcacttgtcctagtaatagtaagc

SEQ ID NO: 39 - DNA, 54 bp, artificial sequence
  Synthetic
ctggcgcgtcccgagacattctcaaaatcaacacttggcctaataatagctagc

SEQ ID NO: 40 - DNA, 54 bp, artificial sequence
  Synthetic
cgggagatgaacccccaaccgtcaaagtctgcacttggcctagtaatagtaagc

SEQ ID NO: 41 - DNA, 54 bp, artificial sequence
  Synthetic
ccggcacactcctcgaccttctcaatgtatccacttgccctagtaataggaagc

SEQ ID NO: 42 - DNA, 54 bp, artificial sequence
  Synthetic
acgaaagggctgatgaccagcccagtcatcgcattcgacgaattacgtattagc

SEQ ID NO: 43 - DNA, 54 bp, artificial sequence
  Synthetic
acgaaggcgctgatgatccccacggcttacgcacttggtttagtaatacgaacc

SEQ ID NO: 44 - DNA, 54 bp, artificial sequence
  Synthetic
ccggcacggtccgagtcattctcaaagtctgcattcggagtactaatagcaagc

SEQ ID NO: 45 - DNA, 54 bp, artificial sequence
  Synthetic
ccggcagggtgcgagacattctcaaagtctgcacttatacatggaatagcggag

SEQ ID NO: 46 - DNA, 54 bp, artificial sequence
  Synthetic
ccgccgtcatgatcccccggcgtccacatcccacttggcctagtaatagtaagc

SEQ ID NO: 47 - DNA, 54 bp, artificial sequence
  Synthetic
ccgtcccaatccgagaccctctcaaagtctacacttcggctagtaataggaagc

SEQ ID NO: 48 - DNA, 54 bp, artificial sequence
  Synthetic
ccggcacggcaccccattccgagaagcctctctttcggcctagttataagaagc

SEQ ID NO: 49 - DNA, 54 bp, artificial sequence
  Synthetic
ccggcagggtgcgagacatactcaaaggctgcacttacactaggaatatcggag

SEQ ID NO: 50 - DNA, 54 bp, artificial sequence
  Synthetic
cgaaggaccgcttcaccgggcgtcgacatccctttcggcttatcaaatgtaagc

SEQ ID NO: 51 - DNA, 54 bp, artificial sequence
  Synthetic
cgcgagctaaacctgcaacccagaagcttctcacttggcctagtaataggtagc

SEQ ID NO: 52 - DNA, 54 bp, artificial sequence
  Synthetic
ccggcacggtccccgcaactgtgaattctctctttcgtcctagtaataggaagc

SEQ ID NO: 53 - DNA, 54 bp, artificial sequence
  Synthetic
ccgctgaggtcgacgagccccgtaacttacccacttgtcctactaagaggtagc

SEQ ID NO: 54 - DNA, 54 bp, artificial sequence
  Synthetic
ccggcacggtccgagacattctaagagtctgaacttggcctagtaatagtaagc

SEQ ID NO: 55 - DNA, 54 bp, artificial sequence
  Synthetic
ccggcacggctgatgagccccctcccaatctcacttgtcctaataatagcacgc

SEQ ID NO: 56 - DNA, 54 bp, artificial sequence
  Synthetic
ccgggtcaatggccgaccagccgccgtgacccattcggcctagtaactagaagc

SEQ ID NO: 57 - DNA, 54 bp, artificial sequence
  Synthetic
ccggcacgctccgagacatactcaaagttcccgcttgggctagtaatagtaagc

SEQ ID NO: 58 - DNA, 54 bp, artificial sequence
  Synthetic
ctggcgcgctccgagacattctcaaagtcgacacttggcctaataatagctaac

SEQ ID NO: 59 - DNA, 54 bp, artificial sequence
  Synthetic
cgacctcaatcgccgaccagccgccgtgacccattcggcctagtaactagaagc

SEQ ID NO: 60 - DNA, 54 bp, artificial sequence
  Synthetic
cgtgagaagatcccccccccctcggtttacgcactgattctagattagcgaaac

SEQ ID NO: 61 - DNA, 54 bp, artificial sequence
  Synthetic
cccgggagcaccatgaccccctcggcttacgcacttgccctagtactagaaggc

SEQ ID NO: 62 - DNA, 54 bp, artificial sequence
  Synthetic
ccggcacggcaccccaaaccgagaagcccctctgttgactaagcaagtattcgc

SEQ ID NO: 63 - DNA, 54 bp, artificial sequence
  Synthetic
acgggacgttgcacccagtctctccgtgacccattcggcctagtaactagaagc

SEQ ID NO: 64 - DNA, 54 bp, artificial sequence
  Synthetic
acgggacgttgcacccagtctctcccaatctcattcggcctagtaactagaagc

SEQ ID NO: 65 - DNA, 54 bp, artificial sequence
  Synthetic
cccgcagaagaggactaagagggccgtgacccattcgacactagatcgagaagc

SEQ ID NO: 66 - DNA, 54 bp, artificial sequence
  Synthetic
ccggcacggcacccccaaccgagaagcctctctttcgtcctagtaataggaagc

SEQ ID NO: 67 - DNA, 54 bp, artificial sequence
  Synthetic
ccggtgtcgggattactgcccggccgtgacgcaaagtcccaatcaactgtaagc

SEQ ID NO: 68 - DNA, 54 bp, artificial sequence
  Synthetic
ccggcacggtccgataccctctcaaagtctgcacttggcctagtaatagctagc

SEQ ID NO: 69 - DNA, 54 bp, artificial sequence
  Synthetic
ccggcacggtctgagacatagtcaaagcccgcacttggcgtagtaatagtaagc

SEQ ID NO: 70 - DNA, 54 bp, artificial sequence
  Synthetic
acgggaggttgcacccagtctctcccaatctcacctgtcctaataatagcgagc

SEQ ID NO: 71 - DNA, 54 bp, artificial sequence
  Synthetic
cgacgtcaatgcccgaccagccgcggtgacccattcggcctagtaactagaagc

SEQ ID NO: 72 - DNA, 54 bp, artificial sequence
  Synthetic
cgccctcaacgatcgaccggccgcgagtctgcactaggcctagtaatagcaagc

SEQ ID NO: 73 - DNA, 54 bp, artificial sequence
  Synthetic
cccggaagatcctccgccttctcaaagtctgcacttacactaggaatagcggag

SEQ ID NO: 74 - DNA, 54 bp, artificial sequence
  Synthetic
ccgccgtcaggattgcccctttcaaagtctgcacttgggctagtaatagcaagc

SEQ ID NO: 75 - DNA, 54 bp, artificial sequence
  Synthetic
ccggcacggcacccccaaccgagaagcctctctttcggcctagtaactagaagc

SEQ ID NO: 76 - DNA, 54 bp, artificial sequence
  Synthetic
ccgggaagctcctagacttccacggcttaggcacttgtgctagtagtacgaagc

SEQ ID NO: 77 - DNA, 54 bp, artificial sequence
  Synthetic
cccggaagatcttagaccgaggtaacatacccattcggcactataacgagaagc

SEQ ID NO: 78 - DNA, 54 bp, artificial sequence
  Synthetic
ccggcacggtcgaagaccccctcggctgtccatttcgggatatcaatcgtaagc

SEQ ID NO: 79 - DNA, 54 bp, artificial sequence
  Synthetic
ccgggaagctccatgaacccctcgactgacgcaattggcctagtaatagcaagc

SEQ ID NO: 80 - DNA, 54 bp, artificial sequence
  Synthetic
ccggcacggcacccccaaccgagaagcctctctttcgacgaatcaagtatgagc

SEQ ID NO: 81 - DNA, 54 bp, artificial sequence
  Synthetic
cccggaagatcctccgccgtagtaacataagcacttagcctcctaatagtcagc

SEQ ID NO: 82 - DNA, 54 bp, artificial sequence
  Synthetic
ccggcctggtcagagacattctcaaactcttcacttggcctagtaatagctagc

SEQ ID NO: 83 - DNA, 54 bp, artificial sequence
  Synthetic
ccggcagggtgcgagacattctcaaagtctgcacttacactaggaatagcggag

SEQ ID NO: 84 - DNA, 54 bp, artificial sequence
  Synthetic
cgagcacattccgagacattctcaaggtctccacttgtcctagtaatagcggag

SEQ ID NO: 85 - DNA, 54 bp, artificial sequence
  Synthetic
cccggaagatcctccgcattctcaaagtctgcacttacactaggaatagcggag

SEQ ID NO: 86 - DNA, 54 bp, artificial sequence
  Synthetic
cccggaagatcctccgccgtagaaacataagcatttagcctgataacgagaatc

SEQ ID NO: 87 - DNA, 54 bp, artificial sequence
  Synthetic
ccgcctcaatggccgaccagccgccgtgacccattcggcctagtaactagaagc

SEQ ID NO: 88 - DNA, 54 bp, artificial sequence
  Synthetic
cccggaagatcgcggtccgaagtagcgttcccacttgtcctagtaatagcggag

SEQ ID NO: 89 - DNA, 54 bp, artificial sequence
  Synthetic
ccggcacggcacccccaaccgagaagcctctcactcgtcctagtaataggaagc

SEQ ID NO: 90 - DNA, 54 bp, artificial sequence
  Synthetic
acgaagcggctcataacccacactgcttattcactagtcctagtaatcgcaagc

SEQ ID NO: 91 - DNA, 54 bp, artificial sequence
  Synthetic
ccggcacggcacccccaaccgagaagcctctctttcggcctagtaatagcaagc

SEQ ID NO: 92 - DNA, 54 bp, artificial sequence
  Synthetic
cccgggaggtcctcgtccgaaggaacgttccgattcgatcctagatcgataagc

SEQ ID NO: 93 - DNA, 54 bp, artificial sequence
  Synthetic
cgtgcacgctccgtgatatattccaagttcccacttggcctagtaataggtagc

SEQ ID NO: 94 - DNA, 54 bp, artificial sequence
  Synthetic
acggcacattccgagacattctcaaggtctccacttgtcctagtaatagcggag

SEQ ID NO: 95 - DNA, 54 bp, artificial sequence
  Synthetic
ccggcacgtgctgagtccctctaaaagtctgtatttggcgtagtaatagtaagc

SEQ ID NO: 96 - DNA, 54 bp, artificial sequence
  Synthetic
cccgcccgtttccctcaacaggcaagcctccctttcggcccgaattagagaagc

SEQ ID NO: 97 - DNA, 54 bp, artificial sequence
  Synthetic
cgggagatgaacccccaaccgagaagcctctcacttggcctagtaatagtaagc

SEQ ID NO: 98 - DNA, 54 bp, artificial sequence
  Synthetic
ccggcacggtccgagacattctcaaagtctgcacttgtcctagtaatagcggag

SEQ ID NO: 99 - DNA, 54 bp, artificial sequence
  Synthetic
acgggacgttgcgcccagtagttcctgatctgacgtgtcgtaataatagcaagc

SEQ ID NO: 100 - DNA, 54 bp, artificial sequence
  Synthetic
ccggcacggtccgagacattctcaaagtctgcacttacactaggaatagcggag

SEQ ID NO: 101 - DNA, 54 bp, artificial sequence
  Synthetic
ccggcatggcatccccaaccgagatgcatctctttcggcctagtaactagatac

SEQ ID NO: 102 - DNA, 54 bp, artificial sequence
  Synthetic
cccgcacattccgagacattctcaaggtctccacttgtcctagtaatagcggag

SEQ ID NO: 103 - DNA, 54 bp, artificial sequence
  Synthetic
ccggctcggtccgacaccctctcaaagtctgcacttggcctagtaatagcgagc

SEQ ID NO: 104 - DNA, 54 bp, artificial sequence
  Synthetic
acgggacgttgcacccagtctctcccaatctcacttggcctagtaatagtaagc

SEQ ID NO: 105 - DNA, 54 bp, artificial sequence
  Synthetic
ccgggacggcaccacccgacctcctcaatctcatttgccctaatagtcgaaagc

SEQ ID NO: 106 - DNA, 54 bp, artificial sequence
  Synthetic
ccggcacggtccgagaaattcttaaagtacgcatagtcccaatcgattgtaagc

SEQ ID NO: 107 - DNA, 54 bp, artificial sequence
  Synthetic
ccggcacggtccgagccccggtcggctctcccacttggcctagtaatagtaagc

SEQ ID NO: 108 - DNA, 54 bp, artificial sequence
  Synthetic
cgggagatgaacccccaaccgagaagccctgcacttggcctagtaatagcaatc

SEQ ID NO: 109 - DNA, 54 bp, artificial sequence
  Synthetic
cccgggagctccgagaccccctcggcttacggatttggcctagtaataggaacc

SEQ ID NO: 110 - DNA, 54 bp, artificial sequence
  Synthetic
aacgccccgggcatcaagtattgtagttaccctttcggcccgaattagataagc

SEQ ID NO: 111 - DNA, 54 bp, artificial sequence
  Synthetic
cgtccttaatgaccgaccaggcgcccggacgcattcggcctagtaactagaagc

SEQ ID NO: 112 - DNA, 54 bp, artificial sequence
  Synthetic
cgggagatgaaccccccatcttcaaagtctccacttgacccagtaataggtagc

SEQ ID NO: 113 - DNA, 54 bp, artificial sequence
  Synthetic
ccgccgtcatgatcccgccccgtaacttacccattcggccctataatagtaagc

SEQ ID NO: 114 - DNA, 54 bp, artificial sequence
  Synthetic
cgacctcaatggccgagcagccgccgtgacccattcggcctagtaactagaagc

SEQ ID NO: 115 - DNA, 54 bp, artificial sequence
  Synthetic
ccggaacggcagccccaacggataatcatctctttcgacctaattacgacaagc

SEQ ID NO: 116 - DNA, 54 bp, artificial sequence
  Synthetic
ccggctagctccgggactttcacaatgtacgcttagtcccaatcaatcgtacgc

SEQ ID NO: 117 - DNA, 54 bp, artificial sequence
  Synthetic
ccacctcaacggccgaccagccgccagtctgcagttggcctagtaatagcaggc

SEQ ID NO: 118 - DNA, 54 bp, artificial sequence
  Synthetic
ccggcacggcacccccgaccgagaagcctctctatcggcctagttatcagaagc

SEQ ID NO: 119 - DNA, 54 bp, artificial sequence
  Synthetic
cgaccccgatggacgaccagccgccgtgacctactcggcctaggagttagaatc

SEQ ID NO: 120 - DNA, 54 bp, artificial sequence
  Synthetic
ccggcacggtcctacaccccttcggcatacgcaattcgccttagaattcgaatc

SEQ ID NO: 121 - DNA, 54 bp, artificial sequence
  Synthetic
ccgccgtcatgatcccgccccggtcggtctccacttgtcctagtaatagcggag

SEQ ID NO: 122 - DNA, 54 bp, artificial sequence
  Synthetic
ccggcacggcacccccaaccgagaagcctcccattcggcctagtaactagaagc

SEQ ID NO: 123 - DNA, 54 bp, artificial sequence
  Synthetic
ccggtgagctcgatgacccaccctgcctgtaaattcgacgatacaagtatcagc

SEQ ID NO: 124 - DNA, 54 bp, artificial sequence
  Synthetic
ccggcagggtgcgagacattctcaaagtctgcacttacactaggaatagcggtc

SEQ ID NO: 125 - DNA, 54 bp, artificial sequence
  Synthetic
ccggcccggtccgagacattctctaagactgcacttggcctagtaatagcaatc

SEQ ID NO: 126 - DNA, 54 bp, artificial sequence
  Synthetic
ccggcacggcaccccccccactcggcttacgcactggtcgtagcattagtaaac

SEQ ID NO: 127 - DNA, 54 bp, artificial sequence
  Synthetic
cgacctcaatggccgaccaggcgccgtgactcattcggcctagtaactagaagc

SEQ ID NO: 128 - DNA, 54 bp, artificial sequence
  Synthetic
acgggacgttgcacccagtctctccctctcccacttggcctagtaatagtaagc

SEQ ID NO: 129 - DNA, 54 bp, artificial sequence
  Synthetic
ccggcacggtccgacacattctcaaagtctccacttgggctagtaatagcaagc

SEQ ID NO: 130 - DNA, 54 bp, artificial sequence
  Synthetic
ccggcacgctccgagacatactcaaagtctgcacttggcctagtaatagtaagc

SEQ ID NO: 131 - DNA, 54 bp, artificial sequence
  Synthetic
ccggcacggtccgagaccccctcaaacctccctttcggcccgaattagagaagc

SEQ ID NO: 132 - DNA, 54 bp, artificial sequence
  Synthetic
ccggcacggtacgagacattctcagagtcagcacttgacctagtaatagcggag

SEQ ID NO: 133 - DNA, 54 bp, artificial sequence
  Synthetic
ccggcacggcacccccaaccgagaagcctctctttcggccttagtacgagaagc

SEQ ID NO: 134 - DNA, 54 bp, artificial sequence
  Synthetic
ccggcacgacacccccaaccgataaacatctctttccgcctacgaatatgaagc

SEQ ID NO: 135 - DNA, 54 bp, artificial sequence
  Synthetic
ccggtgtcgaacccccaaccgagaagcctctctttcggcccgaattagagaatc

SEQ ID NO: 136 - DNA, 54 bp, artificial sequence
  Synthetic
ccggcacggcacccccaaccgagaagcctctctttcggcctagtaataagacgc

SEQ ID NO: 137 - DNA, 54 bp, artificial sequence
  Synthetic
cgacctcaatgcccggcgatccgacgccaaccattcggcctagtaactagaagc

SEQ ID NO: 138 - DNA, 54 bp, artificial sequence
  Synthetic
ccggcacggtgcgagacattctcaaagtctgcacttacactaggaatagcggag

SEQ ID NO: 139 - DNA, 54 bp, artificial sequence
  Synthetic
ctggagacgaatccccccccccccgcttacgcatttggcgtagcactaagaagc

SEQ ID NO: 140 - DNA, 54 bp, artificial sequence
  Synthetic
acgggccgatgatcccgccccggtcgggacggaattggcctagtaatagcaagc

SEQ ID NO: 141 - DNA, 54 bp, artificial sequence
  Synthetic
ccggcacagtctgaggccccgtcggttacgacacttggcctagtaatagtaagc

SEQ ID NO: 142 - DNA, 54 bp, artificial sequence
  Synthetic
aacgccccgggcatcaagtacactgcctattcatttcgcctactaacgagaatc

SEQ ID NO: 143 - DNA, 54 bp, artificial sequence
  Synthetic
cgacctcaatggccgaccagccgccagagctctttcggcccgaattagagaatc

SEQ ID NO: 144 - DNA, 54 bp, artificial sequence
  Synthetic
cccgcccgtttccctcaacaggcaagcctccctttcggcctagtaatagcaagc

SEQ ID NO: 145 - DNA, 54 bp, artificial sequence
  Synthetic
acggcacggtccgagacattctcaaagtctgcacttggcctagtaatagcgggc

SEQ ID NO: 146 - DNA, 54 bp, artificial sequence
  Synthetic
cgacctcaatggccgaccagccgccagagcggacttgtcctagtaatagcggag

SEQ ID NO: 147 - DNA, 54 bp, artificial sequence
  Synthetic
cccgcccctataaggacattctcaaggtctccacttgtcctagtaatagcggag

SEQ ID NO: 148 - DNA, 54 bp, artificial sequence
  Synthetic
cgggagatgaacccccaaccgagaagcctctctttcggcgtaccaagtctaagc

SEQ ID NO: 149 - DNA, 54 bp, artificial sequence
  Synthetic
ccggcacggcacccccaaccgagaagcctctcattcggcctagtaactagaagc

SEQ ID NO: 150 - DNA, 54 bp, artificial sequence
  Synthetic
cctgcacggctgatgacccacctccaaatgggactcgtcctaacaatagcaagc

SEQ ID NO: 151 - DNA, 54 bp, artificial sequence
  Synthetic
ccggcacggcacccccaagcgagaagcctctctttcggcccgaattagagaatc

SEQ ID NO: 152 - DNA, 54 bp, artificial sequence
  Synthetic
ggcgggataaactgcccgtgcggcccatatgcacttacacttctaatagccgag

SEQ ID NO: 153 - DNA, 54 bp, artificial sequence
  Synthetic
ccagcacggcacccgcccccctctgcttacccacttcgcctagtagtagcaagc

SEQ ID NO: 154 - DNA, 54 bp, artificial sequence
  Synthetic
ccggcacgctgtgataccccctctgctctcccacttggcctagtaatagtaagc

SEQ ID NO: 155 - DNA, 54 bp, artificial sequence
  Synthetic
acgaaggagctgatgaccccgtctgctgacgcatttggccttatgatagaagcc

SEQ ID NO: 156 - DNA, 54 bp, artificial sequence
  Synthetic
aacgccccgggcatcaagtattgtagttacccactcggcctagtaatagcggag

SEQ ID NO: 157 - DNA, 54 bp, artificial sequence
  Synthetic
ccggagatgaaccccccattcacaaagtatccacttgccctagtaatagtaagc

SEQ ID NO: 158 - DNA, 54 bp, artificial sequence
  Synthetic
ccggcacggtccgagactccctcggctctctcacttggcctagtaatagtaagc

SEQ ID NO: 159 - DNA, 54 bp, artificial sequence
  Synthetic
cgacctcaatgtccgaccagccgccatgacccgttcggcctagtaactagaagc

SEQ ID NO: 160 - DNA, 54 bp, artificial sequence
  Synthetic
ccgccgtcatgattccggcccgcacgggacggaatcgccggatcaagtataatc

SEQ ID NO: 161 - DNA, 54 bp, artificial sequence
  Synthetic
cgcgagatgagcccccaacagagaagcctctcacttgtcgtagtaataggaagc

SEQ ID NO: 162 - DNA, 54 bp, artificial sequence
  Synthetic
ccggcacggtccgagacattctcaaacctccctttcggcccgaattagagaagc

SEQ ID NO: 163 - DNA, 54 bp, artificial sequence
  Synthetic
ccggcaaggcttccccaaccgaaaagcctctcacttgtcctagtaatgtgacgc

SEQ ID NO: 164 - DNA, 54 bp, artificial sequence
  Synthetic
ctggagatgaacccccccccctcggcttacgcacttgtcctagtaatagtaagc

SEQ ID NO: 165 - DNA, 54 bp, artificial sequence
  Synthetic
cccccccggtccgagacattcgctaagactgcacttggcctagtaatagcaatc

SEQ ID NO: 166 - DNA, 54 bp, artificial sequence
  Synthetic
cgggagatgaacccccaaccgagaagcctctctttcggcgaactaatagcaagc

SEQ ID NO: 167 - DNA, 54 bp, artificial sequence
  Synthetic
ccggcacgctcccagatttactcgaagttcccacttggactagtatcagatagc

SEQ ID NO: 168 - DNA, 54 bp, artificial sequence
  Synthetic
ccggagggtctgatgaccccctcggcttacgcacttgtcctatgatcttgagtc

SEQ ID NO: 169 - DNA, 54 bp, artificial sequence
  Synthetic
acggcacggtccgagacattctcaaagtctgcacttggcctagtaatagcaatc

SEQ ID NO: 170 - DNA, 54 bp, artificial sequence
  Synthetic
ccgccgtcatgatcccgccccgtaacgtctgcacttggcctagtaatagtaagc

SEQ ID NO: 171 - DNA, 54 bp, artificial sequence
  Synthetic
cgacctcaatggccgaccagcggccagtctgcacttcgcctagtaatagcaagc

SEQ ID NO: 172 - DNA, 54 bp, artificial sequence
  Synthetic
cccccccgtttaacccctgctgcctgcctcccacttggcctagtaatagcaatc

SEQ ID NO: 173 - DNA, 54 bp, artificial sequence
  Synthetic
ccggcccggtccgagacattctcaaaccctgcacttggcctagtaatagcaggc

SEQ ID NO: 174 - DNA, 54 bp, artificial sequence
  Synthetic
ccggcacggtccgaggccccctcaagcctccctttcggcccgaattagagaagc

SEQ ID NO: 175 - DNA, 54 bp, artificial sequence
  Synthetic
ccggcacggcacccccaaccgagaagcctctctgttggcctaataatcgtaagc

SEQ ID NO: 176 - DNA, 54 bp, artificial sequence
  Synthetic
ccgaagagctccgagaccccttcggctctcccatttggcctcgtaatagaaatc

SEQ ID NO: 177 - DNA, 54 bp, artificial sequence
  Synthetic
cccgcagaagcccctcaacaggcaagcctccctttcggcccgaattagagaatc

SEQ ID NO: 178 - DNA, 54 bp, artificial sequence
  Synthetic
ccggcacggtccgagacattctcaaagtcttcatttcgcctactaacgagaatc

SEQ ID NO: 179 - DNA, 54 bp, artificial sequence
  Synthetic
cccccccgttccgagacattctcaaggtctccacttgtcctagtaatagcggag

SEQ ID NO: 180 - DNA, 54 bp, artificial sequence
  Synthetic
ccggcaagatcccagatattgtcaaaatctccacttggactggtaatagcaagc

SEQ ID NO: 181 - DNA, 54 bp, artificial sequence
  Synthetic
acggcacggtccgagacattctcaaagtctgcacttggcctagtaataggaacc

SEQ ID NO: 182 - DNA, 54 bp, artificial sequence
  Synthetic
acggcacggtccgagacattctcacgggacggaatcgccgaatcaagtgtaatc

SEQ ID NO: 183 - DNA, 54 bp, artificial sequence
  Synthetic
cgaccttaatggccgaccagcagccgtgacccattcggcctagttactagaagc

SEQ ID NO: 184 - DNA, 54 bp, artificial sequence
  Synthetic
ccggctcgttccgggaccccctctcctctcccacttggcctagtaatagtaagc

SEQ ID NO: 185 - DNA, 54 bp, artificial sequence
  Synthetic
ccggtgtcgggatagacattctcaaagtctgcacttggcctagtaatagtaagc

SEQ ID NO: 186 - DNA, 54 bp, artificial sequence
  Synthetic
ccgtcactcgccgagacatactcaaagttccccctgggcctagtaataggaagc

SEQ ID NO: 187 - DNA, 54 bp, artificial sequence
  Synthetic
cccgggagatcgatgacccaccccgcctatacagttgacgattcaagaaacggc

SEQ ID NO: 188 - DNA, 54 bp, artificial sequence
  Synthetic
acgaggccgctgctgaccccctcagcttacgcacttggcctaataacgggaatc

SEQ ID NO: 189 - DNA, 54 bp, artificial sequence
  Synthetic
ccgtggaactgcgagacattcgcaagttgcgcaaagtcccaaccaattgtaagc

SEQ ID NO: 190 - DNA, 54 bp, artificial sequence
  Synthetic
acgggacgctgcacccagtacctagcactatcacttggcctaataatcgcaagc

SEQ ID NO: 191 - DNA, 54 bp, artificial sequence
  Synthetic
acggagatgtcccctcaacaggcaaggtctccacttgtcctagtaatagcggag

SEQ ID NO: 192 - DNA, 54 bp, artificial sequence
  Synthetic
cgggagatgaacccccaaccgagaagcctctcacttgtcctaggcataggaagc

SEQ ID NO: 193 - DNA, 54 bp, artificial sequence
  Synthetic
cccgcgcggctgaatacgcccctcccaatcaaacttgacctaataatggcatgc

SEQ ID NO: 194 - DNA, 54 bp, artificial sequence
  Synthetic
cgacctcaatggccgaccagccgccaatctgcacttgtcctactaatagctagc

SEQ ID NO: 195 - DNA, 54 bp, artificial sequence
  Synthetic
ccggcacggcacccccaaccgagaagcctcccacttggcctagtaatagtaagc

SEQ ID NO: 196 - DNA, 54 bp, artificial sequence
  Synthetic
ccggcagggtgcgagaccccctcggctctcccacttggcctagtaatagtaagc

SEQ ID NO: 197 - DNA, 54 bp, artificial sequence
  Synthetic
acggcacggtccgagacattctcaaagtctgcacttggcctagtaatagcgatc

SEQ ID NO: 198 - DNA, 54 bp, artificial sequence
  Synthetic
acgaaggattccgagacattctcaaggtctccacttgtcctagtaatagcggag

SEQ ID NO: 199 - DNA, 54 bp, artificial sequence
  Synthetic
cccggaagatcctcgacccacactgcctattcatttcgcctactaacgagaatc

SEQ ID NO: 200 - DNA, 54 bp, artificial sequence
  Synthetic
cgacctcaatggccgaccagccgccgtgacccatacggccctagaacgagaagc

SEQ ID NO: 201 - DNA, 54 bp, artificial sequence
  Synthetic
ccggcacagcccgagtccctctcagagtctgcacttggcctagtgatatcaagc

SEQ ID NO: 202 - DNA, 54 bp, artificial sequence
  Synthetic
ccggcacgctccgagacatactcggctctcccacttggcctagtaatagtaagc

SEQ ID NO: 203 - DNA, 54 bp, artificial sequence
  Synthetic
ccggcagggtgcgagacattctcaaagtctgcacttacactaggaattgcggag

SEQ ID NO: 204 - DNA, 54 bp, artificial sequence
  Synthetic
cccggaagatccgagacattctcaaagtctgcacttggcctagtaatagcggag

SEQ ID NO: 205 - DNA, 54 bp, artificial sequence
  Synthetic
ccggcacggcacccccaaccgagaagcctctctttcggcctaagaattgtaagc

SEQ ID NO: 206 - DNA, 54 bp, artificial sequence
  Synthetic
cccggaagatcctagtccgaggtaacttacccattcggcctagtaatagcaagc

SEQ ID NO: 207 - DNA, 54 bp, artificial sequence
  Synthetic
acccaggtgcggatgacccactcggcttacgtactcggcttaggaatagtaacc

SEQ ID NO: 208 - DNA, 54 bp, artificial sequence
  Synthetic
cgacctcggctgatgaccccctcggcttacgcattcggccctagaacgagaagc

SEQ ID NO: 209 - DNA, 54 bp, artificial sequence
  Synthetic
cccccccggcacccccaaccgagaagcctctctttcggcccgaattagagaatc

SEQ ID NO: 210 - DNA, 54 bp, artificial sequence
  Synthetic
cgggagatgaaaccccaaccgagcagcctctctgtcagataaccaagtctaagc

SEQ ID NO: 211 - DNA, 54 bp, artificial sequence
  Synthetic
ccggcacattccgagacattctcaaggtctccacttggcctagtaatagtaagc

SEQ ID NO: 212 - DNA, 54 bp, artificial sequence
  Synthetic
acggagatgtcccctcaacagggattctacgcaattggccttagaataggactc

SEQ ID NO: 213 - DNA, 54 bp, artificial sequence
  Synthetic
ccggcacagtccgagaccccctcaaccctccctttcggcccgaattagagaagc

SEQ ID NO: 214 - DNA, 54 bp, artificial sequence
  Synthetic
ccgccgtcatgaccccgccccgttcgttccccattcggccctagtatcagaagc

SEQ ID NO: 215 - DNA, 54 bp, artificial sequence
  Synthetic
cccggaagatcctcggccgaagcgacaatcccattcggacctagatcgagaagc

SEQ ID NO: 216 - DNA, 54 bp, artificial sequence
  Synthetic
ccggcacggcacccccaaccgagaagctacccattcggccctagaacgagaagc

SEQ ID NO: 217 - DNA, 54 bp, artificial sequence
  Synthetic
ccggcacggcaccctcccccctcggctgacgcacttggcctagtaatcgaaagc

SEQ ID NO: 218 - DNA, 54 bp, artificial sequence
  Synthetic
actgaaggttcctcacagtctctctcaatctcacttgtccgactaataccaagc

SEQ ID NO: 219 - DNA, 54 bp, artificial sequence
  Synthetic
cgaccgcaatggccgaccagccgccagtctgcacttggcctagtaatagccagc

SEQ ID NO: 220 - DNA, 54 bp, artificial sequence
  Synthetic
ccgccacgtcaccgccagccgataagcctctcactagtccgagtaatagtcagc

SEQ ID NO: 221 - DNA, 54 bp, artificial sequence
  Synthetic
cccggaagatcctcgtccgaagtaacgttcccattcggacttagatcgataagc

SEQ ID NO: 222 - DNA, 54 bp, artificial sequence
  Synthetic
ccggctcgggccacggtcccctcggctttcccacttggcctagtcatagtaagc

SEQ ID NO: 223 - DNA, 54 bp, artificial sequence
  Synthetic
ctggagatgaacccccccccctcggcctacgcacgtatccgagtaacgataagc

SEQ ID NO: 224 - DNA, 54 bp, artificial sequence
  Synthetic
acgaaggagctgattacccacactccctattcatttcgcctaccaacgagaatc

SEQ ID NO: 225 - DNA, 54 bp, artificial sequence
  Synthetic
ctggcaatgtgcgagacatactcaaagtctccacctgacctagtaatagtaagc

SEQ ID NO: 226 - DNA, 54 bp, artificial sequence
  Synthetic
ccggcacggaccgagaccccctcggctctgccacttggcctagtaatagtaagc

SEQ ID NO: 227 - DNA, 54 bp, artificial sequence
  Synthetic
cccgcagaagaggattaagagggccgtgacccattcggcccgaattagagaatc

SEQ ID NO: 228 - DNA, 54 bp, artificial sequence
  Synthetic
ccggcacggtccgagacattctcaaagtctgcacttggcctagtaatagcgatc

SEQ ID NO: 229 - DNA, 54 bp, artificial sequence
  Synthetic
cgggagatgaacccccaaccgagaagcctctctttcggcgaaccaagtcgaatc

SEQ ID NO: 230 - DNA, 54 bp, artificial sequence
  Synthetic
acgggacgttgcacccagtctcccccaatctcagttgtcgattcaatggtatgc

SEQ ID NO: 231 - DNA, 54 bp, artificial sequence
  Synthetic
accaaggtgctaatgaccagccccgcctatacattcgacgcttcaagtattagc

SEQ ID NO: 232 - DNA, 54 bp, artificial sequence
  Synthetic
ccggcacattccgagacattctcaaggtctccacttgtcctagtaatagcgagc

SEQ ID NO: 233 - DNA, 54 bp, artificial sequence
  Synthetic
acgggccgatcccctcaacaggcaagcctccctttcggcccgaattagataagc

SEQ ID NO: 234 - DNA, 54 bp, artificial sequence
  Synthetic
ccggcacgatccgagacgccctcggacctcccacttggcctagcaatattaagc

SEQ ID NO: 235 - DNA, 54 bp, artificial sequence
  Synthetic
acggagatgtcccctcaacaggcaagcctccctttcggcccgaattagataagc

SEQ ID NO: 236 - DNA, 54 bp, artificial sequence
  Synthetic
ccgccgtcatgatcccgccccggtcgggacggaatcggcccgataataagaatc

SEQ ID NO: 237 - DNA, 54 bp, artificial sequence
  Synthetic
ccggcactgcacccccccccctcggcttacccacttggcctagtaatcgaaagc

SEQ ID NO: 238 - DNA, 54 bp, artificial sequence
  Synthetic
cccggaagatcctcgtcccaagtaacgttcccattcggcctagtaatagcaagc

SEQ ID NO: 239 - DNA, 54 bp, artificial sequence
  Synthetic
ccgacacggcacccccaaccgagaagcctctcacttgtcccagtagtacgaagc

SEQ ID NO: 240 - DNA, 54 bp, artificial sequence
  Synthetic
ccggcacggcacccccaaccgagaagcctctctttcggcccgaattagagaagc

SEQ ID NO: 241 - DNA, 54 bp, artificial sequence
  Synthetic
ccggcacggtccgagacattctcaaggtctgcacttggcctagtaatagcggag

SEQ ID NO: 242 - DNA, 54 bp, artificial sequence
  Synthetic
cgggagatgaacccccaaccgagaagcctctctttcggcccgaattatgtaagc

SEQ ID NO: 243 - DNA, 54 bp, artificial sequence
  Synthetic
cgacctcaatggccgaccagccgccagagcggaatcggactagtaatagcaagc

SEQ ID NO: 244 - DNA, 54 bp, artificial sequence
  Synthetic
ccgatgtcatgatctacattcgctaagactgcacttggcctagtaatagcaatc

SEQ ID NO: 245 - DNA, 54 bp, artificial sequence
  Synthetic
cccggaagatcgcggtccagccgccagagcggaatcgccgaatcaacgagaacc

SEQ ID NO: 246 - DNA, 54 bp, artificial sequence
  Synthetic
acgaaggcgctgctgacccacactgcctattcatttcgcctactaacgagaatc

SEQ ID NO: 247 - DNA, 54 bp, artificial sequence
  Synthetic
ccggcacggtccgagacattctcaaagtctgcacttggcctagtaataggaacc

SEQ ID NO: 248 - DNA, 54 bp, artificial sequence
  Synthetic
ccggcacggcacccccaaccgagaagccactcgttcggcgtaagaacgagaagc

SEQ ID NO: 249 - DNA, 54 bp, artificial sequence
  Synthetic
ccggcacggcacccccaaccgagaagcctctccttcggcgtaagaacgagaggc

SEQ ID NO: 250 - DNA, 54 bp, artificial sequence
  Synthetic
ccggcacggcacccccaaccgagaagcctctctttcggcctaagaacgaatatc

SEQ ID NO: 251 - DNA, 54 bp, artificial sequence
  Synthetic
ccgacaaggttcacggtatcctctgcagtcccacttggcctagtaatagcggag

SEQ ID NO: 252 - DNA, 54 bp, artificial sequence
  Synthetic
acgggccgatgcacccttactcacccaatcccgttcgtcccgagttagagaatc

SEQ ID NO: 253 - DNA, 54 bp, artificial sequence
  Synthetic
cccgcccctgttcccggaacactcagcctcccacttggcctagtaatagcggag

SEQ ID NO: 254 - DNA, 54 bp, artificial sequence
  Synthetic
agccagccgaccggtacattctcaaagtctgcacttggcctagtaatagcggag

SEQ ID NO: 255 - DNA, 54 bp, artificial sequence
  Synthetic
ctggcgcggtccgagacattctcaaagactgcacttggcctagtaatagcaatc

SEQ ID NO: 256 - DNA, 54 bp, artificial sequence
  Synthetic
ccggcacggttcgagacatgctcaacgtctgcacgaggcctagtaatagcaagc

SEQ ID NO: 257 - DNA, 54 bp, artificial sequence
  Synthetic
cccccccgtttaacccctgctgcctgcctcccacttggcctagtaacgaggaac

SEQ ID NO: 258 - DNA, 54 bp, artificial sequence
  Synthetic
ccggcacggcacccccaaccgagaagcctcccattcggccctagaacgagaagc

SEQ ID NO: 259 - DNA, 54 bp, artificial sequence
  Synthetic
ccgcccccatgatgctaccgcattacttacccattcggccctaggacgagaagc

SEQ ID NO: 260 - DNA, 54 bp, artificial sequence
  Synthetic
ccggcacggcaccccttaccgaggagcatctcactagtcctagtaataggccgc

SEQ ID NO: 261 - DNA, 54 bp, artificial sequence
  Synthetic
acggcacggtccgagaccccctcaagcctccctttcggcccgaattagagaagc

SEQ ID NO: 262 - DNA, 54 bp, artificial sequence
  Synthetic
cccggaagaacctcgtccgaagtaacgttcccattcggcccgaattagagaagc

SEQ ID NO: 263 - DNA, 54 bp, artificial sequence
  Synthetic
ccggcacggtccgagacattctcaaagactacacttggcgtagtaatagtaatc

SEQ ID NO: 264 - DNA, 54 bp, artificial sequence
  Synthetic
ccggcatggtacgagacattgtctaagtctgcacgtgtcctactaatagcaagc

SEQ ID NO: 265 - DNA, 54 bp, artificial sequence
  Synthetic
cccggaagatcctcgtccgaagtaacgctccctttcggcccgaattagagaatc

SEQ ID NO: 266 - DNA, 54 bp, artificial sequence
  Synthetic
cgcggaagatcctcgtcataaataacgttcctattcgtccctagatcgagaagc

SEQ ID NO: 267 - DNA, 54 bp, artificial sequence
  Synthetic
ccgatgttatgatcttcccccggtatcgacggaaacgacgaatccaatatcagc

SEQ ID NO: 268 - DNA, 54 bp, artificial sequence
  Synthetic
ccggcacggtccgagaccccctcggctctcccattcggcccgaattagagaatc

SEQ ID NO: 269 - DNA, 54 bp, artificial sequence
  Synthetic
ccggcacggtccgagaccctctcgaagtctgcaattggcctagtaatcgcaagc

SEQ ID NO: 270 - DNA, 54 bp, artificial sequence
  Synthetic
aacgccccgggcatcaagtattgtagttaccctttcggcccgaattcgagaagc

SEQ ID NO: 271 - DNA, 54 bp, artificial sequence
  Synthetic
ccagcactatccgagtcccccttggctttcgcagtccgcctagtaatacgaaac

SEQ ID NO: 272 - DNA, 54 bp, artificial sequence
  Synthetic
cccgccgggtgcgagacattctcaaagtctgcacttacactaggaatagcggag

SEQ ID NO: 273 - DNA, 54 bp, artificial sequence
  Synthetic
cccggtgggctcatgtccccctcaggttacgcacttggccttagaattagcacc

SEQ ID NO: 274 - DNA, 54 bp, artificial sequence
  Synthetic
ccggcaaggtttcccacccccacggcttacgcacttggacttagcattcgaatc

SEQ ID NO: 275 - DNA, 54 bp, artificial sequence
  Synthetic
cccggaagatcttctcaacaggcaagcctccctttcggcccgaattagagaatc

SEQ ID NO: 276 - DNA, 54 bp, artificial sequence
  Synthetic
ccgggccggtccgagacattctcaaattctacacttggcttagcaatggtaagc

SEQ ID NO: 277 - DNA, 54 bp, artificial sequence
  Synthetic
ccggtgagagtcgagaccatctcggcgtacgcccttggcctagtaactcgaaac

SEQ ID NO: 278 - DNA, 54 bp, artificial sequence
  Synthetic
ccggcacggcacccccaaccgagaagcctctctttcggcccagtaataggaagc

SEQ ID NO: 279 - DNA, 54 bp, artificial sequence
  Synthetic
ccggcacggtccgagaccccctcggttctcccattcggcccgagttagagcaac

SEQ ID NO: 280 - DNA, 54 bp, artificial sequence
  Synthetic
ccgccgtcaggctcaccctcctcacaatctgcacttggcctaataatagcacgc

SEQ ID NO: 281 - DNA, 54 bp, artificial sequence
  Synthetic
ccggcagggtgcgagacccccgtaactttcccacttggcctagtaatagtaagc

SEQ ID NO: 282 - DNA, 54 bp, artificial sequence
  Synthetic
ccggcacggctgatgaccccctcggcttacgcacttggcctagtaatagtaagc

SEQ ID NO: 283 - DNA, 54 bp, artificial sequence
  Synthetic
cgacgtcaatggccgaccagccgccgtgacccattcggcctagtaactagaagc

SEQ ID NO: 284 - DNA, 54 bp, artificial sequence
  Synthetic
ccggcacggtccgagacattctcaaagtctgcacttggcctaccaagggtaagc

SEQ ID NO: 285 - DNA, 54 bp, artificial sequence
  Synthetic
cgggagatgaacccccaaccgagaagcctctctttcggcccggtaatagaaatc

SEQ ID NO: 286 - DNA, 54 bp, artificial sequence
  Synthetic
ccgggaggagttcccggaacactcagcctcccacttggcctggtattatgaagc

SEQ ID NO: 287 - DNA, 54 bp, artificial sequence
  Synthetic
ccggtgagctactagaccccgtcggctttcgtatttgacgaatccagtacgagc

SEQ ID NO: 288 - DNA, 54 bp, artificial sequence
  Synthetic
ccggctcgccacctccaaccgagaagcctctccttcggcctaagtacgagaagc

SEQ ID NO: 289 - DNA, 54 bp, artificial sequence
  Synthetic
ccggcacggtccgagacattctcaaagtctgcacttggcctaagttcgataatc

SEQ ID NO: 290 - DNA, 54 bp, artificial sequence
  Synthetic
cacgcggaatcggctgaagaggaccgtgactcactcggccctagatcgagaagc

SEQ ID NO: 291 - DNA, 54 bp, artificial sequence
  Synthetic
cgacctcaatggccgaccagccgccagagcggaatcgccgaatcaacagcaagc

SEQ ID NO: 292 - DNA, 54 bp, artificial sequence
  Synthetic
ccggcatggaacccccaaccgagaagcctctctttcggcccgaattagagaatc

SEQ ID NO: 293 - DNA, 54 bp, artificial sequence
  Synthetic
cccgcagaagaggattaagagggccgtgactctttcggcccgaattagagaatc

SEQ ID NO: 294 - DNA, 54 bp, artificial sequence
  Synthetic
ccgggacgatccgagacattctcaaagtctgcacttgggctagtaatagtaagc

SEQ ID NO: 295 - DNA, 54 bp, artificial sequence
  Synthetic
cgaccgagatgatcccgccccgtaacttacccattcggccctagaacgagaagc

SEQ ID NO: 296 - DNA, 54 bp, artificial sequence
  Synthetic
ccgccgtggtcccctcaacaggcaagcctccctttcggcccgaattagagaatc

SEQ ID NO: 297 - DNA, 54 bp, artificial sequence
  Synthetic
ccgaagagctccgagacattcgcaacttacccattgggccctagaacgagaagc

SEQ ID NO: 298 - DNA, 54 bp, artificial sequence
  Synthetic
cgacctcaatggccgagccccgtaacttacccattcggccctagaacgagaagc

SEQ ID NO: 299 - DNA, 54 bp, artificial sequence
  Synthetic
cccggaagatcctccgcacaggcaagcctccctttcggcccgaattagagaatc

SEQ ID NO: 300 - DNA, 54 bp, artificial sequence
  Synthetic
ccggaggcgctgatgaccccctcggcatacgcaagtcgcctaagccttagatgc

SEQ ID NO: 301 - DNA, 54 bp, artificial sequence
  Synthetic
ccggcacggtccgcgacattgtcaaagtctgtacttggcctagtaatactatgc

SEQ ID NO: 302 - DNA, 54 bp, artificial sequence
  Synthetic
cgggagatgaacccccaaccgagaagcctctctttcggcccgtcaacgagaacc

SEQ ID NO: 303 - DNA, 54 bp, artificial sequence
  Synthetic
ccggtaagatgctgttccgagataagttacccagtcggccttagaacgtgatgc

SEQ ID NO: 304 - DNA, 54 bp, artificial sequence
  Synthetic
ccggcacggtccgagacattctcaaagtctgcactcggactagtaatagcaagc

SEQ ID NO: 305 - DNA, 54 bp, artificial sequence
  Synthetic
ccgacacggttcatggccggcgtccacctcccattcggcctgagatcgataatc

SEQ ID NO: 306 - DNA, 54 bp, artificial sequence
  Synthetic
ccggtgtcgggattactgcccggccgtgacggagtcgccgaaccaattgtaagc

SEQ ID NO: 307 - DNA, 54 bp, artificial sequence
  Synthetic
cccggaagaacctcgtccgaatcaaagtctgcacttggcctagtaatagcgatc

SEQ ID NO: 308 - DNA, 54 bp, artificial sequence
  Synthetic
ccggcacggtccgagacattctcaaagctccctttcggcccgaattagagaagc

SEQ ID NO: 309 - DNA, 54 bp, artificial sequence
  Synthetic
cgacctcaatggccgaccagccgccgtgacccattcggcctcgtaactagaagc

SEQ ID NO: 310 - DNA, 54 bp, artificial sequence
  Synthetic
ccggcacggtccgagacattctcaaagtcttcacttggcctagtaatagcgatc

SEQ ID NO: 311 - DNA, 54 bp, artificial sequence
  Synthetic
cctgcatggtccgagaccccctcggctctcccacttggcctagtaatagtaagc

SEQ ID NO: 312 - DNA, 54 bp, artificial sequence
  Synthetic
ccgccgttgaacccccaaccgagaagcctctctttcggcccgaattagagaatc

SEQ ID NO: 313 - DNA, 54 bp, artificial sequence
  Synthetic
ccggcacgctccgagacatactcaaagttcccacttggcctagtaataggtagc

SEQ ID NO: 314 - DNA, 54 bp, artificial sequence
  Synthetic
ccggcgcggttcgagaccccctcggctcttcctttcgtcccgagttagagaatc

SEQ ID NO: 315 - DNA, 54 bp, artificial sequence
  Synthetic
cccggaagatccttgacccacactgcctattcatttcgcctactaacgagaatc

SEQ ID NO: 316 - DNA, 54 bp, artificial sequence
  Synthetic
cccgcccggtccgagacattctcaaagtctgcacttggcctagtaatagcggag

SEQ ID NO: 317 - DNA, 54 bp, artificial sequence
  Synthetic
ccggcacattccgagacattctctaggtcgccacttgtcctagtaatagcggag

SEQ ID NO: 318 - DNA, 54 bp, artificial sequence
  Synthetic
cccgaccgcttcccacaacaggcaagcctccctttcggcccgaattagagaatc

SEQ ID NO: 319 - DNA, 54 bp, artificial sequence
  Synthetic
cccagacaattctcgtccgtagtaacattccctttcggcccgaattagagaagc

SEQ ID NO: 320 - DNA, 54 bp, artificial sequence
  Synthetic
acgaagttgctcctgaccccctcgggttacgcacttggcctagtatcgaagaac

SEQ ID NO: 321 - DNA, 54 bp, artificial sequence
  Synthetic
ccggcacggctgatgacccccctcccaatattactagtccttataatagcgacc

SEQ ID NO: 322 - DNA, 54 bp, artificial sequence
  Synthetic
cgcggaagctcctcgtccgaagtaacgttcccatacggccctatatcgagaagc

SEQ ID NO: 323 - DNA, 54 bp, artificial sequence
  Synthetic
acgggccggaccgagacatccttaaattctggacttggcctagtaacgagtaac

SEQ ID NO: 324 - DNA, 54 bp, artificial sequence
  Synthetic
ccggcacggcaacccgaaccgagaagcctctctcttggcctaataataccaggc

SEQ ID NO: 325 - DNA, 54 bp, artificial sequence
  Synthetic
ccggcacggcacccccaaccgagaagcctctctttcggcctagtaacgagaagc

SEQ ID NO: 326 - DNA, 54 bp, artificial sequence
  Synthetic
ccgacgcaatgttctcaacaggcaagcctccctttcggcccgaattagagaatc

SEQ ID NO: 327 - DNA, 54 bp, artificial sequence
  Synthetic
cgtgagatgtcgatgacccacactgcctattcatttcgcctactaacgagaatc

SEQ ID NO: 328 - DNA, 54 bp, artificial sequence
  Synthetic
ccggcacggcacccccaaccgagaagcctctctttcggcccgaattagagactc

SEQ ID NO: 329 - DNA, 54 bp, artificial sequence
  Synthetic
cccgcagaagcgtcgtccgaagtaagcctccctttcggcccgaattagagaatc

SEQ ID NO: 330 - DNA, 54 bp, artificial sequence
  Synthetic
ccggcacggtccgagacattctcaaagtctgcacttggcctatcaacgagaatc

SEQ ID NO: 331 - DNA, 54 bp, artificial sequence
  Synthetic
ccgacaagatcctcgtccgaagtaacgttcccacttgtcctagtaatagcggag

SEQ ID NO: 332 - DNA, 54 bp, artificial sequence
  Synthetic
ccgccgtcaggattactccccggccgggtcccattcggcccgaattagagaatc

SEQ ID NO: 333 - DNA, 54 bp, artificial sequence
  Synthetic
cgggcacgctcctaagcttaggcaaagttcccacttggcttagtaatacgaagc

SEQ ID NO: 334 - DNA, 54 bp, artificial sequence
  Synthetic
ccggcacggtccgagacattctcaaagtctgcacttggcctaggtttagcaagc

SEQ ID NO: 335 - DNA, 54 bp, artificial sequence
  Synthetic
ccggcacggtccgagacattctcaaagtctgcacttggcctagtatcgagaatc

SEQ ID NO: 336 - DNA, 54 bp, artificial sequence
  Synthetic
ccggcacggtccgggaccccctcaagcctccctttcggcccgaattagagaagc

SEQ ID NO: 337 - DNA, 54 bp, artificial sequence
  Synthetic
ccgacacggtccgagacattctcaaagtctgcacttggcctagtaatagcgatc

SEQ ID NO: 338 - DNA, 54 bp, artificial sequence
  Synthetic
cccgcagaagaggattaagagggccgtgctgcacttggcctagtaatagcggag

SEQ ID NO: 339 - DNA, 54 bp, artificial sequence
  Synthetic
ccggcacggtccgagacattctcaaagccttcacttggcctagtaatagtaagc

SEQ ID NO: 340 - DNA, 54 bp, artificial sequence
  Synthetic
cgacctcaatggccgacccgccgccgtgactcattcggccgagtaactagaagc

SEQ ID NO: 341 - DNA, 54 bp, artificial sequence
  Synthetic
acgggacgttgcacccagtctctcccgatctcacttgtccggtcaattgtaagc

SEQ ID NO: 342 - DNA, 54 bp, artificial sequence
  Synthetic
atgacactacgaggtaagtcccgacgtctccctttcggcccaagatcgagaatc

SEQ ID NO: 343 - DNA, 54 bp, artificial sequence
  Synthetic
cgggagatgaacccccaaccgagaagcctctcattccgccctataacgagaacc

SEQ ID NO: 344 - DNA, 54 bp, artificial sequence
  Synthetic
ctggcacggtccgagaccccctcaagcctccctttcggcccgaattagagaagc

SEQ ID NO: 345 - DNA, 54 bp, artificial sequence
  Synthetic
ccggcacggtccgagaccccctcgtctctcccacttggcctagtaatagtaagc

SEQ ID NO: 346 - DNA, 54 bp, artificial sequence
  Synthetic
ccgacaagatcctcgtccgagagaagcctctctttcggcccgaattagagaatc

SEQ ID NO: 347 - DNA, 54 bp, artificial sequence
  Synthetic
acggcgacgtcccctcaacaggcaagcctccctttcggcccgaattagagaatc

SEQ ID NO: 348 - DNA, 54 bp, artificial sequence
  Synthetic
acgggtctgtcccccaagtattgtagttaccctttcggcccgaattagagaatc

SEQ ID NO: 349 - DNA, 54 bp, artificial sequence
  Synthetic
ccggcacggctcagggtactctcaaagtctgcacttacactaggaatagcggag

SEQ ID NO: 350 - DNA, 54 bp, artificial sequence
  Synthetic
ccggctcggtccgagtcattcacaacctctgcacttggccaagtaatcgcaagc

SEQ ID NO: 351 - DNA, 54 bp, artificial sequence
  Synthetic
cgtgagatgtcccttcgatagtcaagcttccctttaggcccgaattcgagaagc

SEQ ID NO: 352 - DNA, 54 bp, artificial sequence
  Synthetic
acagtacgttgcacccagtctctcccaatctcacgtgtcctaataatcgcaagc

SEQ ID NO: 353 - DNA, 54 bp, artificial sequence
  Synthetic
ctgcgcaaacgctctaccgcatctacactcccgttcggcccgagatcgagaatc

SEQ ID NO: 354 - DNA, 54 bp, artificial sequence
  Synthetic
ccaacacggcacccccaaccgagaagcctccctttcggcccgaattagagaatc

SEQ ID NO: 355 - DNA, 54 bp, artificial sequence
  Synthetic
ccgccgtcaggattactccccggccgggtcccattcggcccgaattagagaagc

SEQ ID NO: 356 - DNA, 54 bp, artificial sequence
  Synthetic
ccagtaggtgttcccgcatcatacagccgcccacttggcctagtaatagtaagc

SEQ ID NO: 357 - DNA, 54 bp, artificial sequence
  Synthetic
acgggacgttgttcccagtctcgcccactctcacttgtcgaattaagaggtatc

SEQ ID NO: 358 - DNA, 54 bp, artificial sequence
  Synthetic
ccggcacggctgatgacccccctcccaatctcacaggtccgaataatagcaagc

SEQ ID NO: 359 - DNA, 54 bp, artificial sequence
  Synthetic
ccggcacattccgagacattctcaaggtctccacttacactaggaatagcggag

SEQ ID NO: 360 - DNA, 54 bp, artificial sequence
  Synthetic
ccggcacgctccgaggcattctcaaagtctgcactcgacgaatcaagtatcagc

SEQ ID NO: 361 - DNA, 54 bp, artificial sequence
  Synthetic
acgaagggctccgagacattcgctaagactgcacttggcctagtaatagcggag

SEQ ID NO: 362 - DNA, 54 bp, artificial sequence
  Synthetic
cgacctcaatggccgaccagccgccgtgacccattcggcccgaattagagaatc

SEQ ID NO: 363 - DNA, 54 bp, artificial sequence
  Synthetic
ccgcacgggctaatgaccccctcggcttacgcacatcgcccgattatagaaagc

SEQ ID NO: 364 - DNA, 54 bp, artificial sequence
  Synthetic
ccgacaagatcctcgtccgaactaacgttcccattcggccctagatcgagatgc

SEQ ID NO: 365 - DNA, 54 bp, artificial sequence
  Synthetic
ccggcacggtccgagacattctcaaagtctgcacttggcctaggaacagcgatc

SEQ ID NO: 366 - DNA, 54 bp, artificial sequence
  Synthetic
cccgcacggtccgatacatactctaagtctgcacttggcctagtaactcgaagc

SEQ ID NO: 367 - DNA, 54 bp, artificial sequence
  Synthetic
ccggcaaggctcctccccccctcggcatacgctattggcccatcgattaaacgc

SEQ ID NO: 368 - DNA, 54 bp, artificial sequence
  Synthetic
cgacctcaatggccgaccagccgccgtgacccacttggccgagtaacgagaaac

SEQ ID NO: 369 - DNA, 54 bp, artificial sequence
  Synthetic
cccgcagaagcgtcgtccaaagtaacgttccgaatcagccctagatcgagaagc

SEQ ID NO: 370 - DNA, 54 bp, artificial sequence
  Synthetic
ccggcagaatgcgagacccccgtaacttacccaatcggccatagaacgagaagc

SEQ ID NO: 371 - DNA, 54 bp, artificial sequence
  Synthetic
cccgcagaagaggattaagagggccgttacccactcggcccgagaacgagtagc

SEQ ID NO: 372 - DNA, 54 bp, artificial sequence
  Synthetic
acggcacggtccgagacattctcaaagtctgcacttggcctactaatagcggag

SEQ ID NO: 373 - DNA, 54 bp, artificial sequence
  Synthetic
ccgtagggctccgagacccccgccctaatttcacttgtcgaatcaatagcagcc

SEQ ID NO: 374 - DNA, 54 bp, artificial sequence
  Synthetic
acggagatgtcccctcaacaggcaagcctccctttcggcccgaattcgataatc

SEQ ID NO: 375 - DNA, 54 bp, artificial sequence
  Synthetic
cccgcccctataaggcaacaggcaagcctccctttcggcccgaattagagaagc

SEQ ID NO: 376 - DNA, 54 bp, artificial sequence
  Synthetic
ccggcacgctccgagacatactcaaagttcccacttggcctagtaatagtaagc

SEQ ID NO: 377 - DNA, 54 bp, artificial sequence
  Synthetic
cccggaacatgatcccgccccggtcgggacggaatcgccgaatcaacgagaatc

SEQ ID NO: 378 - DNA, 54 bp, artificial sequence
  Synthetic
ccggcacggtccgagaccctctcaaagtctgcacttggcctagtaatagcgatc

SEQ ID NO: 379 - DNA, 54 bp, artificial sequence
  Synthetic
acgggccgttgctcgtccgcagtaacgttccctttcggcccgaattagagaatc

SEQ ID NO: 380 - DNA, 54 bp, artificial sequence
  Synthetic
ccggcacggctgatgactcccctcccgatagcacttgcccgaataatcgcaagc

SEQ ID NO: 381 - DNA, 54 bp, artificial sequence
  Synthetic
ccggcatgctccgaaacctcctcggccgtcccacttggccgagtaagtgtaatc

SEQ ID NO: 382 - DNA, 54 bp, artificial sequence
  Synthetic
ccggcacggtcagagacatggtcaaagtctgcacttggcctagtaatagtaagc

SEQ ID NO: 383 - DNA, 54 bp, artificial sequence
  Synthetic
ctggcgcggtccaagaccttcgcaaagtgtgcatttggcctaataatagcacgc

SEQ ID NO: 384 - DNA, 54 bp, artificial sequence
  Synthetic
ccggcatggtccgggactttctcaaagtctgccctgggcctagtaatagcaagc

SEQ ID NO: 385 - DNA, 54 bp, artificial sequence
  Synthetic
ccggcacggcacccccaaccgagaagcctctcttgtggcctgataatcgcatgc

SEQ ID NO: 386 - DNA, 54 bp, artificial sequence
  Synthetic
acgggacgttgcacccagtctctccgttacccatacggccctagaacgagaagc

SEQ ID NO: 387 - DNA, 54 bp, artificial sequence
  Synthetic
ccggcacggtccgagaccctctcaaagtctgcacttggcctagtaatagcaatc

SEQ ID NO: 388 - DNA, 54 bp, artificial sequence
  Synthetic
cccgcccctataaggacataactcagcctcccacttggcctagtaatagcggag

SEQ ID NO: 389 - DNA, 54 bp, artificial sequence
  Synthetic
cccgcccgtttccctcaacaggcaacctattcatttcgcctactaacgagaatc

SEQ ID NO: 390 - DNA, 54 bp, artificial sequence
  Synthetic
ccggcacattccgagacattcgctatgtctacacttgtcctagtaataggacgc

SEQ ID NO: 391 - DNA, 54 bp, artificial sequence
  Synthetic
cccggaagatcctcgtccgaagtaacgttcccattcggacctagatcgagaagc

SEQ ID NO: 392 - DNA, 54 bp, artificial sequence
  Synthetic
cccggaagatcctcttccgaagtaacgttcccattcgtccctagatcgagaagc

SEQ ID NO: 393 - DNA, 54 bp, artificial sequence
  Synthetic
cgggagcaatggccgaccagccgccgtgacccattcggcctagtaactagaagc

SEQ ID NO: 394 - DNA, 54 bp, artificial sequence
  Synthetic
cccccccgtttaacccctgctgcctgcctcccacttggcctagtaatagtaagc

SEQ ID NO: 395 - DNA, 54 bp, artificial sequence
  Synthetic
acggagtgtcccgagacattctcaaagtctgcacttggcctagtaatagcggag

SEQ ID NO: 396 - DNA, 54 bp, artificial sequence
  Synthetic
cgacctctatggccgaccagccgccgtgacccacttggcctagtaacgagatac

SEQ ID NO: 397 - DNA, 54 bp, artificial sequence
  Synthetic
ctggagatgagcgagacccccgtaacttacccattcggccctagaacgagaagc

SEQ ID NO: 398 - DNA, 54 bp, artificial sequence
  Synthetic
ccgccgtcagacccccaaccgagaagcctctctttcggcccgaattagagaatc

SEQ ID NO: 399 - DNA, 54 bp, artificial sequence
  Synthetic
cgacctcaatggccgaccagccgccgtgacccatacggccgtagatcgagaagc

SEQ ID NO: 400 - DNA, 54 bp, artificial sequence
  Synthetic
cccgcccgtttccctcaacaggcaagcctccctttcggcccgaattaggagagc

SEQ ID NO: 401 - DNA, 54 bp, artificial sequence
  Synthetic
ccggcacggtccgagacattcgcaacttacccattcggccctagaacgagaagc

SEQ ID NO: 402 - DNA, 54 bp, artificial sequence
  Synthetic
ccggtacggtccgagacattctcaaagtctgcacttggcctagtaatagtaagc

SEQ ID NO: 403 - DNA, 54 bp, artificial sequence
  Synthetic
acgaagggtctgctgagccacaccgcttgcgcacttgtcctagttatacgaagc

SEQ ID NO: 404 - DNA, 54 bp, artificial sequence
  Synthetic
cccggcaggcagatgtccctatcggcttacgcacttggcctaataactcggatc

SEQ ID NO: 405 - DNA, 54 bp, artificial sequence
  Synthetic
ccggagagccacgagacattcgcctttgacgcaaagtccgaatcaattggaggc

SEQ ID NO: 406 - DNA, 54 bp, artificial sequence
  Synthetic
aacgccccgggcatcaagtattgtagttacccacttcgcctgctaatagcaatc

SEQ ID NO: 407 - DNA, 54 bp, artificial sequence
  Synthetic
ccggcacattccgagaccccctcaagcctccctttcggcccgaattagagaagc

SEQ ID NO: 408 - DNA, 54 bp, artificial sequence
  Synthetic
ccgggtaactccgaggccttcgctacaaatgcacttggcctagtaatagtggag

SEQ ID NO: 409 - DNA, 54 bp, artificial sequence
  Synthetic
ccggcacgatccgagacattctcaaagtctgcacttggcctagtaatagcaatc

SEQ ID NO: 410 - DNA, 54 bp, artificial sequence
  Synthetic
ccggcgagctccgagaccccagttacttacccattcggccctagaacgagaagc

SEQ ID NO: 411 - DNA, 54 bp, artificial sequence
  Synthetic
cgacctcaatggccgaccagccgccatgacccattcggcctcgtaactagaagc

SEQ ID NO: 412 - DNA, 54 bp, artificial sequence
  Synthetic
acgaagggactgatgaccccctcggcttgcgcacttgtccaagtaacgacaagc

SEQ ID NO: 413 - DNA, 54 bp, artificial sequence
  Synthetic
cccgcccgtttccctcaacaggcaagcctccctttcggcccgaattagagaatc

SEQ ID NO: 414 - DNA, 54 bp, artificial sequence
  Synthetic
caggagatgatccccccaatctcaaagtctccacttgtcctagtaatcggaagc

SEQ ID NO: 415 - DNA, 54 bp, artificial sequence
  Synthetic
gggtgcgcggttcccggagcccccagcatcccacttgtcctagtaatagccgag

SEQ ID NO: 416 - DNA, 54 bp, artificial sequence
  Synthetic
cccgcacggtccgagacattctcaaagtctgcacttggcctagtaatagtaagc

SEQ ID NO: 417 - DNA, 54 bp, artificial sequence
  Synthetic
ccgccgtcaggatcaccccccggaaagtctcctttcggcccgaattagagaagc

SEQ ID NO: 418 - DNA, 54 bp, artificial sequence
  Synthetic
cgtgagatgtcccttcgatagtcaagcttccctttcggcccgaattagagaagc

SEQ ID NO: 419 - DNA, 54 bp, artificial sequence
  Synthetic
ccggcacggcccgagaccccctcggctctcccacttggcctagtaatagtaagc

SEQ ID NO: 420 - DNA, 54 bp, artificial sequence
  Synthetic
ctggcacggcacccccaaccgagatgcctctcactggtcccagtaacaggatgc

SEQ ID NO: 421 - DNA, 54 bp, artificial sequence
  Synthetic
ccggcacggtgcgacacatcctcaaagtctgcacttggcctagtaatagtaagc

SEQ ID NO: 422 - DNA, 54 bp, artificial sequence
  Synthetic
cccccccggtccgagaccccctcggctctcccacttggcctagtaatagtaagc

SEQ ID NO: 423 - DNA, 54 bp, artificial sequence
  Synthetic
cgggagatgaacccccaaccgagaagcctctcattccgcccgaattagagaatc

SEQ ID NO: 424 - DNA, 54 bp, artificial sequence
  Synthetic
ccggcacggtccgagacattctcaaagtctgcacttggcctaccaatgcgaatc

SEQ ID NO: 425 - DNA, 54 bp, artificial sequence
  Synthetic
cccggaagatcccctcaacaggcaagcctccctttcggcccgaattagagaatc

SEQ ID NO: 426 - DNA, 54 bp, artificial sequence
  Synthetic
acgggccgatgcacccttactcacccaatcccacttcccgaatcaagtggaatc

SEQ ID NO: 427 - DNA, 54 bp, artificial sequence
  Synthetic
ccggcatggtcccctcaacaggcaagcctccctttcggcccgaattagagaatc

SEQ ID NO: 428 - DNA, 54 bp, artificial sequence
  Synthetic
ccgggacggtccgagaccccctcggctctcccacttggcctagtaatagtaagc

SEQ ID NO: 429 - DNA, 54 bp, artificial sequence
  Synthetic
ccggcacattcggagacattctcaaggtctccacttggcctagtaatagcggag

SEQ ID NO: 430 - DNA, 54 bp, artificial sequence
  Synthetic
acgggccgatgcacccttactcacccaatcccaatcgccgaatcaagtgtaatc

SEQ ID NO: 431 - DNA, 54 bp, artificial sequence
  Synthetic
ccggcatggtcccctcaaaaggcaaacctccctttcggcccgaattagagaatc

SEQ ID NO: 432 - DNA, 54 bp, artificial sequence
  Synthetic
ccggcacggtccgagaccccctcaagcctccctttcggcgaatcaagtatcagc

SEQ ID NO: 433 - DNA, 54 bp, artificial sequence
  Synthetic
ccggcacggtccgagaccccctcgggcctcccacttggcctagtaatagcggag

SEQ ID NO: 434 - DNA, 54 bp, artificial sequence
  Synthetic
acggcacggtccgagacattctcaaagtctgcaattggcctactaatagcggag

SEQ ID NO: 435 - DNA, 54 bp, artificial sequence
  Synthetic
ccggcacggcacccccaaccgagaagcctctctttcggcctcaggacgagtagc

SEQ ID NO: 436 - DNA, 54 bp, artificial sequence
  Synthetic
acgggccgatcgcctcaacaggctagcctcactttcggcccgaattagagaatc

SEQ ID NO: 437 - DNA, 54 bp, artificial sequence
  Synthetic
ctggcgcggtccgagccattcgcaaactcagcacttggcctaataatcgcaagc

SEQ ID NO: 438 - DNA, 54 bp, artificial sequence
  Synthetic
acgggccgttgcacccatcaggcaagcctccctttcggcccgaattagagaatc

SEQ ID NO: 439 - DNA, 54 bp, artificial sequence
  Synthetic
ccggcaatgtccgagacgtactcaaagtttctacttaacctaggaatcgcaagc

SEQ ID NO: 440 - DNA, 54 bp, artificial sequence
  Synthetic
acggagatgtcccctcaacaggcaagcctccctttcggcccgaattagaggatc

SEQ ID NO: 441 - DNA, 54 bp, artificial sequence
  Synthetic
ctcttaagaacctcgtccgaagtaacgttcccattcggccctatatcgataagc

SEQ ID NO: 442 - DNA, 54 bp, artificial sequence
  Synthetic
cccggaagatcctccgccgtagtaaccctccctttcggcccgaattagagaagc

SEQ ID NO: 443 - DNA, 54 bp, artificial sequence
  Synthetic
cccgcccgtataaccatatactggtctgacggagaggtagatccaagcgttcgc

SEQ ID NO: 444 - DNA, 54 bp, artificial sequence
  Synthetic
ccgccgtcatgatcccgccccggtccgttccctttcggcccgaattagagaatc

SEQ ID NO: 445 - DNA, 54 bp, artificial sequence
  Synthetic
cgacctcaatggccgaccagcggccgtgacccatttggccctagttcgagcagc

SEQ ID NO: 446 - DNA, 54 bp, artificial sequence
  Synthetic
cccggaagatcctcgtccgcagtaacgtctccacttgtcctagtaatagcggag

SEQ ID NO: 447 - DNA, 54 bp, artificial sequence
  Synthetic
cccccccgtttaaccccattctcaaagtctgcacttggcctagtaatagcggag

SEQ ID NO: 448 - DNA, 54 bp, artificial sequence
  Synthetic
acggcacggtccgagacattctcaaagtctgcactcggccctagatcgagaagc

SEQ ID NO: 449 - DNA, 54 bp, artificial sequence
  Synthetic
ctggcgcggtccgagacattcgcgaagtcaacactaggcctattaacagcaagc

SEQ ID NO: 450 - DNA, 54 bp, artificial sequence
  Synthetic
cgggagatgaacccccaaccgagaagcctctcattccgccctataagtctaagc

SEQ ID NO: 451 - DNA, 54 bp, artificial sequence
  Synthetic
ccgacaaggtcccctcaacaggcaagcctccctttcggcccgaattagagaagc

SEQ ID NO: 452 - DNA, 54 bp, artificial sequence
  Synthetic
cgggagatgaacccccaaccgagaagcctctctgtcgtcgaaccaagtctaagc

SEQ ID NO: 453 - DNA, 54 bp, artificial sequence
  Synthetic
cgggagatgaacccccaaccgagaagcctctctttcggcccgacaagtctaagc

SEQ ID NO: 454 - DNA, 54 bp, artificial sequence
  Synthetic
ccgccgtcatgatcccgccccagtcgggtaggaatcgccgaatcaacgagaatc

SEQ ID NO: 455 - DNA, 54 bp, artificial sequence
  Synthetic
acgggacgttccgagacccccgtaacttacccattcggccctagaacgagaagc

SEQ ID NO: 456 - DNA, 54 bp, artificial sequence
  Synthetic
ccggcacggtcaagcccaggagaagttgtcgcaaagtcccaatcaactgtaagc

SEQ ID NO: 457 - DNA, 54 bp, artificial sequence
  Synthetic
ccggcacggctgatgacccacactgcctattcatttcgcctactaacgagaatc

SEQ ID NO: 458 - DNA, 54 bp, artificial sequence
  Synthetic
ccgccgtcaggatcaccccccggccgggaccctttcggcccgaattagagaatc

SEQ ID NO: 459 - DNA, 54 bp, artificial sequence
  Synthetic
ccgccgtcaggatcacccccctcaaagtctgcacttggcctagtaatagcaatc

SEQ ID NO: 460 - DNA, 54 bp, artificial sequence
  Synthetic
ccgacaaggttcacggtatacactgcctattcatttcgcctactaacgagaatc

SEQ ID NO: 461 - DNA, 54 bp, artificial sequence
  Synthetic
ccggcacgatccgagacaccctcaagcctccctttcggcccgaattagagaagc

SEQ ID NO: 462 - DNA, 54 bp, artificial sequence
  Synthetic
ccggcacggtccgagacattctcaaagccttcacttggcctagtaatagcaatc

SEQ ID NO: 463 - DNA, 54 bp, artificial sequence
  Synthetic
cgacctcaatggccgaccagccgccagtctgcacttggcctagtaatagcaatc

SEQ ID NO: 464 - DNA, 54 bp, artificial sequence
  Synthetic
cccgcagagtcccctcaacaggcaagcctccctttcggcccgaattagagaatc

SEQ ID NO: 465 - DNA, 54 bp, artificial sequence
  Synthetic
acgggacgttgcacccagtctctccccctccctttcggcccgaattagagaagc

SEQ ID NO: 466 - DNA, 54 bp, artificial sequence
  Synthetic
acgggccgatgcacccttactcacccaatcccgttcgtcccgagttcgagaagc

SEQ ID NO: 467 - DNA, 54 bp, artificial sequence
  Synthetic
ccagcacggtccgagaccccctcggttctcccacttggcctagtaatagtaagc

SEQ ID NO: 468 - DNA, 54 bp, artificial sequence
  Synthetic
acgaaagggctcatggcctgctcggcttacgcacgtggcctagtaatagacatc

SEQ ID NO: 469 - DNA, 54 bp, artificial sequence
  Synthetic
cgtgagatgtcccctacccacactgcctattcatttcgcctactaacgagaatc

SEQ ID NO: 470 - DNA, 54 bp, artificial sequence
  Synthetic
ccgatgtcatgatcttccccctcaaagtctgcacttggcctagtaatagtaagc

SEQ ID NO: 471 - DNA, 54 bp, artificial sequence
  Synthetic
ccgccgtcatgatcccgccccggtcgcgacggaatcgccgaatcaacgagaatc

SEQ ID NO: 472 - DNA, 54 bp, artificial sequence
  Synthetic
atggcacgacgagttgaataagtaaagttcccattcctccctacttcgagaagc

SEQ ID NO: 473 - DNA, 54 bp, artificial sequence
  Synthetic
acgaaggtgctgatgacctcctcggcttacgcacttggcctaggttagcgaaac

SEQ ID NO: 474 - DNA, 54 bp, artificial sequence
  Synthetic
ccggtcagctccgagtcctcctcagcttacggactgggcctagtaagtcggaac

SEQ ID NO: 475 - DNA, 54 bp, artificial sequence
  Synthetic
ccgatgtcatgatcttcccccggtagcgacggaaacgacgtagtaatagcaagc

SEQ ID NO: 476 - DNA, 54 bp, artificial sequence
  Synthetic
aacgccccgggcatcaagtattgtagttaccctttcggcccgacaagtctaagc

SEQ ID NO: 477 - DNA, 54 bp, artificial sequence
  Synthetic
acggagatgtcccctcaacaggcaagcctccctttcggcccgaattcgagaaac

SEQ ID NO: 478 - DNA, 54 bp, artificial sequence
  Synthetic
acgaaacggtccgagaccccctcaagcctccctttcggcccgaattagagaagc

SEQ ID NO: 479 - DNA, 54 bp, artificial sequence
  Synthetic
ccgatgagctcggagaccacctcggcttacgcactttacgtagtcgctagaatc

SEQ ID NO: 480 - DNA, 54 bp, artificial sequence
  Synthetic
ccgccgtcatgatcccgccccgtaagcctctctttcggcccgaattagagaatc

SEQ ID NO: 481 - DNA, 54 bp, artificial sequence
  Synthetic
ccggcacggcacccccacccctcggctctcccacttggcctagtaatagtaagc

SEQ ID NO: 482 - DNA, 54 bp, artificial sequence
  Synthetic
ccggcacggcacccccaaccgagccgtgacccattcggcctagtaactagaagc

SEQ ID NO: 483 - DNA, 54 bp, artificial sequence
  Synthetic
ccggcacggcacccacccccctcggcttacgcacttgtcctagtaatagcacgc

SEQ ID NO: 484 - DNA, 54 bp, artificial sequence
  Synthetic
ccggcacggtccgagaccccctcaaagtctgcacttggcctagtaatagtaagc

SEQ ID NO: 485 - DNA, 54 bp, artificial sequence
  Synthetic
cccggaagatcctcgtccgaagtaagcctccctttcggcccgaattagagaatc

SEQ ID NO: 486 - DNA, 54 bp, artificial sequence
  Synthetic
ccggtgtcgggattactgcccggccgtgacggagtcgccgaaccaagtaatagc

SEQ ID NO: 487 - DNA, 54 bp, artificial sequence
  Synthetic
acgggacgttgcacccagtctgctaagatcccattcggccctagaacgagaagc

SEQ ID NO: 488 - DNA, 54 bp, artificial sequence
  Synthetic
ccggctcggccgtagacaccctcaagcctatctttcggcccgaatttgagaagc

SEQ ID NO: 489 - DNA, 54 bp, artificial sequence
  Synthetic
cccccccgtttaacccctgctgcctgcctcccacttggcctagtaacgtgaaac

SEQ ID NO: 490 - DNA, 54 bp, artificial sequence
  Synthetic
ccggcacggtccgagaccctctcaaagtccgcccttggcctagcaatagcaggc

SEQ ID NO: 491 - DNA, 54 bp, artificial sequence
  Synthetic
ccgccgtcaggatcaccccccggaaagtctgcacttggcctagtaactaggatc

SEQ ID NO: 492 - DNA, 54 bp, artificial sequence
  Synthetic
cccgcagaagaggattaagagggccgtgacccattcggcctagtaatagcgatc

SEQ ID NO: 493 - DNA, 54 bp, artificial sequence
  Synthetic
ccggcacggcacccccaaccgataacttacccattcggccctagaacgagaagc

SEQ ID NO: 494 - DNA, 54 bp, artificial sequence
  Synthetic
ccgggaagatgcccgacgagccgcggtgacccacttggcctagtaatagggagc

SEQ ID NO: 495 - DNA, 54 bp, artificial sequence
  Synthetic
ctggagataaacccccatcggagaagccgcggacttggcctagtcataggaagc

SEQ ID NO: 496 - DNA, 54 bp, artificial sequence
  Synthetic
ccggcacggtccgagacattctcaaagactgcacttggcctagtaatagcggag

SEQ ID NO: 497 - DNA, 54 bp, artificial sequence
  Synthetic
acggaggtgtcccctcaacaggcaagcctccctttcggcccgaattagagaatc

SEQ ID NO: 498 - DNA, 54 bp, artificial sequence
  Synthetic
ccgacatggtcccctcaccagggaagcctccctttcggcccgaattagagaatc

SEQ ID NO: 499 - DNA, 54 bp, artificial sequence
  Synthetic
ccgccgcaatgttctcaacaggcaagcctccctttcggcccgaattagagaatc

SEQ ID NO: 500 - DNA, 54 bp, artificial sequence
  Synthetic
ccggcacggtccgagacattctcaaagtcccctttcggcccgaattagagaagc

SEQ ID NO: 501 - DNA, 54 bp, artificial sequence
  Synthetic
ccgccgtcatgatcccgccccggtcgggacggaatcgccgtatcaagtgtaatc

SEQ ID NO: 502 - DNA, 54 bp, artificial sequence
  Synthetic
cccggaagaacctcgtccgaagcaagcctccctttcggcccgaattagagaatc

SEQ ID NO: 503 - DNA, 54 bp, artificial sequence
  Synthetic
ccggagagcttgatgaccccctcggtttaggcacttggccaatgaacgtgacgc

SEQ ID NO: 504 - DNA, 54 bp, artificial sequence
  Synthetic
acggagcaccactgcagaatccgacgtctccctttcggcccaagatcgagaatc

SEQ ID NO: 505 - DNA, 54 bp, artificial sequence
  Synthetic
ccgccgtcaggatcacccccgagaagcctctctttcggcccgaattagagaatc

SEQ ID NO: 506 - DNA, 54 bp, artificial sequence
  Synthetic
cctgcccggtccgagacattctcaaactctgcacttggcccagtaataccaagc

SEQ ID NO: 507 - DNA, 54 bp, artificial sequence
  Synthetic
cccgcccgtttccctcgacaggcaagcctccctttcggcccgaattagagaatc

SEQ ID NO: 508 - DNA, 54 bp, artificial sequence
  Synthetic
caggcatggaacccccacccgtgaagcctctctttgcgcctaataacatcgagc

SEQ ID NO: 509 - DNA, 54 bp, artificial sequence
  Synthetic
ccggcacggtccgagacattctcaaagtctgcacttggcctagcaagggtaagc

SEQ ID NO: 510 - DNA, 54 bp, artificial sequence
  Synthetic
ccggcacggcacccccaaccgagaaagatcccattcggccctagaacgagaagc

SEQ ID NO: 511 - DNA, 54 bp, artificial sequence
  Synthetic
ccggcagggtgcgagacattctcaaagtctgcacttacactaggaatagcgggc

SEQ ID NO: 512 - DNA, 54 bp, artificial sequence
  Synthetic
acgggtctgtccgagacattctcaaagtctgcacttggcctagtaatagcgatc

SEQ ID NO: 513 - DNA, 54 bp, artificial sequence
  Synthetic
cccggaagatcctcgtccgcagtaacgttccctttcggcccgaattagagaagc

SEQ ID NO: 514 - DNA, 54 bp, artificial sequence
  Synthetic
aacgccccgggcatcaagtattgtagttaccctttcggcccgaattggagaatc

SEQ ID NO: 515 - DNA, 54 bp, artificial sequence
  Synthetic
cccggaaggaacccccaaccgagaagcctctctttcggcccgaattagagaatc

SEQ ID NO: 516 - DNA, 54 bp, artificial sequence
  Synthetic
ccggaaccgctgatgacccccctcgcaatctcactggtccttataatagcaggc

SEQ ID NO: 517 - DNA, 54 bp, artificial sequence
  Synthetic
cccgcgatgtcccctcaacaggcaagcctccctttcggcccgaattagagaatc

SEQ ID NO: 518 - DNA, 54 bp, artificial sequence
  Synthetic
ccgagaagctccgagacccccttggcttacgcactaggcctcccaacgaaacgc

SEQ ID NO: 519 - DNA, 54 bp, artificial sequence
  Synthetic
ccgccgtcatgatcccgccccgcaagcctccctttcggcccgaattagagaatc

SEQ ID NO: 520 - DNA, 54 bp, artificial sequence
  Synthetic
acgaaggtgctgatgacccgcactgcctattcatttcgcctaccaacgagaatc

SEQ ID NO: 521 - DNA, 54 bp, artificial sequence
  Synthetic
ccgcggagctccataaccccctcggcttacgcaattggcctaggagtaggacgc

SEQ ID NO: 522 - DNA, 54 bp, artificial sequence
  Synthetic
ccggaactgtccaaaaccccgtcggctcacgcacgggacctagtaatagcaacc

SEQ ID NO: 523 - DNA, 54 bp, artificial sequence
  Synthetic
cgggcacggcacccccaaccgagaagcctctctttcggcccgaattagagaatc

SEQ ID NO: 524 - DNA, 54 bp, artificial sequence
  Synthetic
ccgccgtcatgatcccgccccgtaacttacccattcggcctagtaatagcaagc

SEQ ID NO: 525 - DNA, 54 bp, artificial sequence
  Synthetic
aacgccccgggcatcaagcaggcaagcctccctttcggcccgaattagagaatc

SEQ ID NO: 526 - DNA, 54 bp, artificial sequence
  Synthetic
cgggagatgaacgcccaaccgagaaccctctttgtcggcgaaccaactcttagc

SEQ ID NO: 527 - DNA, 54 bp, artificial sequence
  Synthetic
ccggcacggtccgaaaccccctcggctctcccacttggcctagtaatagtaagc

SEQ ID NO: 528 - DNA, 54 bp, artificial sequence
  Synthetic
ccggcacggtccgagacccccttccacaacccattcggccctagaacgagaagc

SEQ ID NO: 529 - DNA, 54 bp, artificial sequence
  Synthetic
ccgccagggctgatgtccacctcggcttacgctcatgtcctatgaatcgcaaac

SEQ ID NO: 530 - DNA, 54 bp, artificial sequence
  Synthetic
ccgccgtcatgatcccgccccggtcggctctctttcggcccgaattagagaatc

SEQ ID NO: 531 - DNA, 54 bp, artificial sequence
  Synthetic
ccggcacggctgatgaccccctcggcttacccattcggccctagaacgagaagc

SEQ ID NO: 532 - DNA, 54 bp, artificial sequence
  Synthetic
ccgacacggttcatggtatcctcaaagtctgcacttggcctagtaatagcggag

SEQ ID NO: 533 - DNA, 54 bp, artificial sequence
  Synthetic
acggagtgtctttcgtccgcagtaacgttccctttcggcccgaattagagaatc

SEQ ID NO: 534 - DNA, 54 bp, artificial sequence
  Synthetic
ccggcacggcatccccacctttaggctaacggacttgtcctagtaatagaccgc

SEQ ID NO: 535 - DNA, 54 bp, artificial sequence
  Synthetic
ccggcacggtccgagaccccctcaagcctccctttcggcctagtaatagcaagc

SEQ ID NO: 536 - DNA, 54 bp, artificial sequence
  Synthetic
cccggaagatcctccgccgtagtaacgtctgcacttggcctagtaatagcggag

SEQ ID NO: 537 - DNA, 54 bp, artificial sequence
  Synthetic
cgggagatgaacccccaaccgagaaccctctctttcggcccgaattagagaatc

SEQ ID NO: 538 - DNA, 54 bp, artificial sequence
  Synthetic
cgggagatgaacccccaaccgagaagcctctcacttgtcctagcaataggaatc

SEQ ID NO: 539 - DNA, 54 bp, artificial sequence
  Synthetic
cgggagatgagcaccggcacctcggctgaggcacttgccctaggaatagcaagc

SEQ ID NO: 540 - DNA, 54 bp, artificial sequence
  Synthetic
acgggccgatcccctcaacaggcagcctattcatttcgcctactaacgagaatc

SEQ ID NO: 541 - DNA, 54 bp, artificial sequence
  Synthetic
cccccccggtccgagacattctcaaagtctgcacttggcctagtaatagtaagc

SEQ ID NO: 542 - DNA, 54 bp, artificial sequence
  Synthetic
ccggcacggtccgtgacattctcaaagtctgcacttggtctagtaatacgaaac

SEQ ID NO: 543 - DNA, 54 bp, artificial sequence
  Synthetic
ccggcacggctcagggtactctttacagacgctttcggcccgaattagagaatc

SEQ ID NO: 544 - DNA, 54 bp, artificial sequence
  Synthetic
ccgccgtcaggatcaccccccggccgggacggagacgcccctagatcgagaagc

SEQ ID NO: 545 - DNA, 54 bp, artificial sequence
  Synthetic
cgggagatgaacccccaaccgagaagcctctctttcggcgaaccaagtctaggc

SEQ ID NO: 546 - DNA, 54 bp, artificial sequence
  Synthetic
ccgggcatggactcccccccctcggcttacgcacttgtgctagccatgagaacc

SEQ ID NO: 547 - DNA, 54 bp, artificial sequence
  Synthetic
cgaccgcaatgttctcaagaggcaagcctccctttcggcccgaattagagaatc

SEQ ID NO: 548 - DNA, 54 bp, artificial sequence
  Synthetic
ccgccgtcgtccgagaccccctcaagcctccctttcggcccgaattagagaagc

SEQ ID NO: 549 - DNA, 54 bp, artificial sequence
  Synthetic
acaaaggaggtgatgacgagccctgcccacacattcgccgaatcaagtattagc

SEQ ID NO: 550 - DNA, 54 bp, artificial sequence
  Synthetic
cgaccctaaggaccgacccgccgccgcgacccattccgccctagatcgagaagc

SEQ ID NO: 551 - DNA, 54 bp, artificial sequence
  Synthetic
acgagtgggctgtcgaccccctcggcttacgcatttggccccgttatagaagac

SEQ ID NO: 552 - DNA, 54 bp, artificial sequence
  Synthetic
cccggaagatcctagtccgaggtcacttacccatttggccgtagaacgagaagc

SEQ ID NO: 553 - DNA, 54 bp, artificial sequence
  Synthetic
cccggaagatcctccgccgtcgtaacataagcacttagcctaagttcgagaatc

SEQ ID NO: 554 - DNA, 54 bp, artificial sequence
  Synthetic
caggcgatgaacccccatccgagccgccaccctgttggcctagtaagtagtacc

SEQ ID NO: 555 - DNA, 54 bp, artificial sequence
  Synthetic
ccggcacggcacccccaagcgagaggcctctctctcggccccgtaactagagac

SEQ ID NO: 556 - DNA, 54 bp, artificial sequence
  Synthetic
ctagcgcggtccgagacattctcaaagtcaagacttgcccgaattatagcaagc

SEQ ID NO: 557 - DNA, 54 bp, artificial sequence
  Synthetic
ccgggtaggacagagacgttcaataagtacccagtcggccttataacgagaaac

SEQ ID NO: 558 - DNA, 54 bp, artificial sequence
  Synthetic
ccgccgtcatgatcccgcccagtaacgttccctttcggcccgaattagagaatc

SEQ ID NO: 559 - DNA, 54 bp, artificial sequence
  Synthetic
cccggaagatcctcgtccgactcaaagtctgcacttggcctagtaatagcgatc

SEQ ID NO: 560 - DNA, 54 bp, artificial sequence
  Synthetic
acgggccggcacccccaaccgagaagcctctctttcggcccgaattagagaatc

SEQ ID NO: 561 - DNA, 54 bp, artificial sequence
  Synthetic
cggcgcagctgcgagaaattcgctaagtctgcacttgtcctagtaatacgaagc

SEQ ID NO: 562 - DNA, 54 bp, artificial sequence
  Synthetic
aacgccccgggcatcaagtattgtagttaccctttcggcccgaattagagaagc

SEQ ID NO: 563 - DNA, 54 bp, artificial sequence
  Synthetic
cccgaaagatcgcccggaacactcagcctcccacttggcctagtaatagcggag

SEQ ID NO: 564 - DNA, 54 bp, artificial sequence
  Synthetic
ccgccgtcatgatcccgccccggtcgggacggaatcggcccgaattagagaatc

SEQ ID NO: 565 - DNA, 54 bp, artificial sequence
  Synthetic
ccggtgtagtgattccgtcccgtggattacccatacggccctagaacgatacgc

SEQ ID NO: 566 - DNA, 54 bp, artificial sequence
  Synthetic
ccggcacggtccgagacccccttccacatcccattcggccctagaacgagaagc

SEQ ID NO: 567 - DNA, 54 bp, artificial sequence
  Synthetic
ccggcacggcacccccaaccgagaagcctctctttcggcccggtaacgagaaac

SEQ ID NO: 568 - DNA, 54 bp, artificial sequence
  Synthetic
ccggagatgtcccctcaacaggcaagcctccctttcggcccgaattagagaatc

SEQ ID NO: 569 - DNA, 54 bp, artificial sequence
  Synthetic
ccggcacggtccgagaccccctcggctctcccacttggcctagtaatagtaagc

SEQ ID NO: 570 - DNA, 54 bp, artificial sequence
  Synthetic
cccggaagatcctcgtccgaagtaacgttcccattcggcccgaattagagaatc

SEQ ID NO: 571 - DNA, 54 bp, artificial sequence
  Synthetic
ccggcacggtccgagactgtcctgatcctccctttcggcccgaattagagaatc

SEQ ID NO: 572 - DNA, 54 bp, artificial sequence
  Synthetic
cccgcatggtcccctcaacaggcaagcctccctttcggcccgaattagagaatc

SEQ ID NO: 573 - DNA, 54 bp, artificial sequence
  Synthetic
ccggcagggtgcgagacattctcaagcctccctttcggcccgaattagagaatc

SEQ ID NO: 574 - DNA, 54 bp, artificial sequence
  Synthetic
acgaagggcctgatgaccccctcggcttacggattccgccctagatcgagaagc

SEQ ID NO: 575 - DNA, 54 bp, artificial sequence
  Synthetic
ccgatgtcatgatcttcccccggtagcctccctttcggcccgaattagagaagc

SEQ ID NO: 576 - DNA, 54 bp, artificial sequence
  Synthetic
ccgggaagatgatgttccgagatcagttaaccagtcggcccaagaacgtgaagc

SEQ ID NO: 577 - DNA, 54 bp, artificial sequence
  Synthetic
cccggaagattgatgaccccctcggcttacgcagacgccgaaccaagggtaagc

SEQ ID NO: 578 - DNA, 54 bp, artificial sequence
  Synthetic
cccggaagctcctagtccgagctaacttacccattcgaccctagaacgagtagc

SEQ ID NO: 579 - DNA, 54 bp, artificial sequence
  Synthetic
cccccccgtttaaccccacgaggagtatacccattcggccctagaacgagaagc

SEQ ID NO: 580 - DNA, 54 bp, artificial sequence
  Synthetic
ccggcacggctgatgacccacactgcctattcatttcgcctaccaacgagaatc

SEQ ID NO: 581 - DNA, 54 bp, artificial sequence
  Synthetic
acgaagggccggatgaccccctcggcttacggcgacgccgtactaatagaaacc

SEQ ID NO: 582 - DNA, 54 bp, artificial sequence
  Synthetic
atggcacaacgaggtaacgaagtaacgttcgcacttacactaggaatagcggag

SEQ ID NO: 583 - DNA, 54 bp, artificial sequence
  Synthetic
ccgaggagctccgagaccccgaccccaatcacacttgtcctactaatcgtaagc

SEQ ID NO: 584 - DNA, 54 bp, artificial sequence
  Synthetic
ccgccgtcatgatcccgcccctcggcttacgcagacgccgaaccaagggtaagc

SEQ ID NO: 585 - DNA, 54 bp, artificial sequence
  Synthetic
acgggccggtcccctcaacaggcaagcctccctttcggcccgaattagagaatc

SEQ ID NO: 586 - DNA, 54 bp, artificial sequence
  Synthetic
ctggacaagaaccccccgccgtgcagcctcccacttggcctggtaatagggagc

SEQ ID NO: 587 - DNA, 54 bp, artificial sequence
  Synthetic
ccggcaggctgcgaaacccccgtaacttacccattcggccctagaacgagaagc

SEQ ID NO: 588 - DNA, 54 bp, artificial sequence
  Synthetic
ctggagatgagcccccctccctccgcttacgcactaggcctagtaacgtgaaac

SEQ ID NO: 589 - DNA, 54 bp, artificial sequence
  Synthetic
ccgacaagatcctcgtcccacactgcctattcatttcgcctactaacgagaatc

SEQ ID NO: 590 - DNA, 54 bp, artificial sequence
  Synthetic
cccggaagatcctcgtcccaagtaacgtctgcacttggcctagtaatagcgatc

SEQ ID NO: 591 - DNA, 54 bp, artificial sequence
  Synthetic
ccgccgtcatgatcccgccccggtcggtacccattcggccctagaagtatcagc

SEQ ID NO: 592 - DNA, 54 bp, artificial sequence
  Synthetic
ccggcactgtccgagatatactataagtcttcactcggcctaggaatacgaagc

SEQ ID NO: 593 - DNA, 54 bp, artificial sequence
  Synthetic
cccggaagatcctcgtccgaagtaaccttcccaatcggaccttgatcgataagc

SEQ ID NO: 594 - DNA, 54 bp, artificial sequence
  Synthetic
ccggcacggcacccccaaccgagaagcctctctctcggccgagtaactagaaac

SEQ ID NO: 595 - DNA, 54 bp, artificial sequence
  Synthetic
ccggcacggtcccctcaacaggcaagcctccctttcggcccgaattagagaatc

SEQ ID NO: 596 - DNA, 54 bp, artificial sequence
  Synthetic
acggcacggtccgagacattctcaaagtatgcacttggcctagtaatagcggag

SEQ ID NO: 597 - DNA, 54 bp, artificial sequence
  Synthetic
ccggcgtcatgatcccgtcccgtgcgttacccatacggccctagaacgagaagc

SEQ ID NO: 598 - DNA, 54 bp, artificial sequence
  Synthetic
cgaccgcaatgttctcaaccgtcaagcctccctttcggcccgaattagagaatc

SEQ ID NO: 599 - DNA, 54 bp, artificial sequence
  Synthetic
acggcacggtccgagacattctcaaagtctgctttcggcccgaattagagaagc

SEQ ID NO: 600 - DNA, 54 bp, artificial sequence
  Synthetic
cccggaagatcccctcaacaggcaagcctccctttcggcccgaattagagaagc

SEQ ID NO: 601 - DNA, 54 bp, artificial sequence
  Synthetic
atggcatggtcccctcaacaggcaagcctccctttcggcccgaattagagaatc

SEQ ID NO: 602 - DNA, 54 bp, artificial sequence
  Synthetic
ccgccgtcttgatcccgcccccgtctggacggaatcaccgaatcaacgagaacc

SEQ ID NO: 603 - DNA, 54 bp, artificial sequence
  Synthetic
cagggtatgaaccgccccccctcggcttacgctcttcgccttgtatcgagaaac

SEQ ID NO: 604 - DNA, 54 bp, artificial sequence
  Synthetic
ccggcacggcactcccaacccagaagcctctctttcggcccgaattagagaatc

SEQ ID NO: 605 - DNA, 54 bp, artificial sequence
  Synthetic
acgaagatgctgatgacccaccccgcctagacactcgaagaatcgggtattggc

SEQ ID NO: 606 - DNA, 54 bp, artificial sequence
  Synthetic
cgggagatgaacccccaaccgagaagcctctctttcggcgaaccaagtctaaac

SEQ ID NO: 607 - DNA, 54 bp, artificial sequence
  Synthetic
ccggcacggtccgagacattctcaaagtctgcacttggcctagtaatagtagag

SEQ ID NO: 608 - DNA, 54 bp, artificial sequence
  Synthetic
ccgccgtcatgatcccgccccggtcgggacggaatcgccgaatcaacgagaatc

SEQ ID NO: 609 - DNA, 54 bp, artificial sequence
  Synthetic
ccggcacattccgagacattctcaaggtctccacttgtcctactaatagcggag

SEQ ID NO: 610 - DNA, 54 bp, artificial sequence
  Synthetic
acgaaggtgctgatgacccacactgcttattcatttcgcctaccaacgagaatc

SEQ ID NO: 611 - DNA, 54 bp, artificial sequence
  Synthetic
ccctgaacatacgcgaccgaagtaacagtcactttcggccctagttcgagaagc

SEQ ID NO: 612 - DNA, 54 bp, artificial sequence
  Synthetic
acggagcgatggccgaccagccgccgtgacccattcggcctagtaactagaagc

SEQ ID NO: 613 - DNA, 54 bp, artificial sequence
  Synthetic
ccgacaaggttcacggtatcctctggcctccctttcggcccgaattagagaagc

SEQ ID NO: 614 - DNA, 54 bp, artificial sequence
  Synthetic
ccggcacggcacacccagccgagaagcctctctttcggcccgaattagagaatc

SEQ ID NO: 615 - DNA, 54 bp, artificial sequence
  Synthetic
ccggcacggtccgagacattgtcaaagtctgcacttggccctagaacgatcagc

SEQ ID NO: 616 - DNA, 54 bp, artificial sequence
  Synthetic
cgggagatgaacccccaaccggtaacttacccattcggccctagaacgagaagc

SEQ ID NO: 617 - DNA, 54 bp, artificial sequence
  Synthetic
ccggcacggcacccccaaccgagaagcctctctttcggcccagtaatagtaagc

SEQ ID NO: 618 - DNA, 54 bp, artificial sequence
  Synthetic
cgacctcaatggccgacattctcaaagtctgcacttggcctagtaatagtaagc

SEQ ID NO: 619 - DNA, 54 bp, artificial sequence
  Synthetic
ccggcatggtccgagcccccctagactaacggagacgtcgaaacatcgagaatc

SEQ ID NO: 620 - DNA, 54 bp, artificial sequence
  Synthetic
ccggtgtcgggattactgcccggccgcctccctttcggcccgaattagagaatc

SEQ ID NO: 621 - DNA, 54 bp, artificial sequence
  Synthetic
cccggaagatcctcgtccaaattaacgttcccattcggccctagatcgagaaac

SEQ ID NO: 622 - DNA, 54 bp, artificial sequence
  Synthetic
ccgccgtcatgatcccgccccggtagttaccctttcggcccgaattagagaatc

SEQ ID NO: 623 - DNA, 54 bp, artificial sequence
  Synthetic
aacgccccgggcatcaagtattgtagttaccctttcggcccgatttcgagaatc

SEQ ID NO: 624 - DNA, 54 bp, artificial sequence
  Synthetic
cgggagatgaacccccaaccgagaagcctctctttcggcgtagtaatagcaagc

SEQ ID NO: 625 - DNA, 54 bp, artificial sequence
  Synthetic
ccggaacgctccgagacatactcaaagttcccacttggccgagtaataggaagc

SEQ ID NO: 626 - DNA, 54 bp, artificial sequence
  Synthetic
ctgccccagtgcgagacattctcaatctctgcacttggcctagtaatagtaagc

SEQ ID NO: 627 - DNA, 54 bp, artificial sequence
  Synthetic
caggctcggcttatgaccccctcggcttacgcactttgcctagtaacgggaatc

SEQ ID NO: 628 - DNA, 54 bp, artificial sequence
  Synthetic
cccgcagaagaggattaagagggccgtgacccattcggccctagatcgagaacc

SEQ ID NO: 629 - DNA, 54 bp, artificial sequence
  Synthetic
ccggcacggtccgagaccccctcatgcctccctttcggcccgaattagagaagc

SEQ ID NO: 630 - DNA, 54 bp, artificial sequence
  Synthetic
ccgggaagctccgagacattcgctaagactgcaattggcctagtaataggcggc

SEQ ID NO: 631 - DNA, 54 bp, artificial sequence
  Synthetic
cggcggagctccgagacattcgctagttcccctgtcggccttagaacgagtagc

SEQ ID NO: 632 - DNA, 54 bp, artificial sequence
  Synthetic
ccgccgtcatgatcccgccccgggaaactcccattcggcccgagaacgagaacc

SEQ ID NO: 633 - DNA, 54 bp, artificial sequence
  Synthetic
ccgccgtcaggatcacccccctcaaagtctgcacttggcctagtatcgagaagc

SEQ ID NO: 634 - DNA, 54 bp, artificial sequence
  Synthetic
acggagatgtcccctcaacaggcaagcctccctttcggcccgaattagagaagc

SEQ ID NO: 635 - DNA, 54 bp, artificial sequence
  Synthetic
ccggcacggtccgagaccccctcaagcctccctttcggcccgaattagagaatc

SEQ ID NO: 636 - DNA, 54 bp, artificial sequence
  Synthetic
cccgcagaagcggattacgagagtgcctattcatttcgcctactaacgagaatc

SEQ ID NO: 637 - DNA, 54 bp, artificial sequence
  Synthetic
ccggtaagatcctgttccgagataagttacccagttggccttagaacgagcagc

SEQ ID NO: 638 - DNA, 54 bp, artificial sequence
  Synthetic
ccgccgtcatgatcccgccccggtcgcctcccacttggcctagtaatagcggag

SEQ ID NO: 639 - DNA, 54 bp, artificial sequence
  Synthetic
ccgggaagatcctagacattctcaaagtctgcacttggcctagtaatagtaagc

SEQ ID NO: 640 - DNA, 54 bp, artificial sequence
  Synthetic
ccggaagtgctgatggccccctcggcgtacactgttgggctagcaactaggatc

SEQ ID NO: 641 - DNA, 54 bp, artificial sequence
  Synthetic
ccggcagggtgcgagacccccgtccgtgacccattcggcctagtaactagaagc

SEQ ID NO: 642 - DNA, 54 bp, artificial sequence
  Synthetic
ccggcacattgcatgatcccctatgctttcgcacttggcctagcagtagtaaac

SEQ ID NO: 643 - DNA, 54 bp, artificial sequence
  Synthetic
ccgggtatctcatcgcccggcgtccctgacgcacttggccttgtaatagacgac

SEQ ID NO: 644 - DNA, 54 bp, artificial sequence
  Synthetic
acggcacggtcagagacattcgcatagtctgcacttggcctagtaatagcggag

SEQ ID NO: 645 - DNA, 54 bp, artificial sequence
  Synthetic
ctggagaggaacccccatcggagccgcctcccacttggcctattaatagggagc

SEQ ID NO: 646 - DNA, 54 bp, artificial sequence
  Synthetic
ccggcacggcacccccaaccgagaagcctctctttcggcccgataatagaaatc

SEQ ID NO: 647 - DNA, 54 bp, artificial sequence
  Synthetic
ccggcacggtccgagaccccctcggctgacggaatcgccgaatcaacgagaatc

SEQ ID NO: 648 - DNA, 54 bp, artificial sequence
  Synthetic
cccggaagatcctccgccgtaggtagttaccctttcggcccgaattagagaatc

SEQ ID NO: 649 - DNA, 54 bp, artificial sequence
  Synthetic
ccggcacggtccgagaccccctcggctctcccgcttggcctagtaatagtaagc

SEQ ID NO: 650 - DNA, 54 bp, artificial sequence
  Synthetic
ccggcacattccgagacattctcaaggtctccacttgtcctagtaatagcggag

SEQ ID NO: 651 - DNA, 54 bp, artificial sequence
  Synthetic
cccgcagaagttcccggaacactcagcctcccacttggcctagtaatagcggag

SEQ ID NO: 652 - DNA, 54 bp, artificial sequence
  Synthetic
ccggcacgttccgagaccctctcaaagtcttcacttcgaccagtaatagcgagc

SEQ ID NO: 653 - DNA, 54 bp, artificial sequence
  Synthetic
ccggcacggtccaagacattctcacagtctgcacttggcctaagcacgagaagc

SEQ ID NO: 654 - DNA, 54 bp, artificial sequence
  Synthetic
ccggcactgtccgagaccccctcggctctcccacttggcctagtaatagtaagc

SEQ ID NO: 655 - DNA, 54 bp, artificial sequence
  Synthetic
cgacctcaatggccaaccagccgccgtgacccattcggccgtagatcgagaagc

SEQ ID NO: 656 - DNA, 54 bp, artificial sequence
  Synthetic
ccggcacggcggatcacccccctcccaattacacttgacctaataatcgagagc

SEQ ID NO: 657 - DNA, 54 bp, artificial sequence
  Synthetic
acggagatgtcccctcaacaggcagtatacccattcggccctagaacgagaagc

SEQ ID NO: 658 - DNA, 54 bp, artificial sequence
  Synthetic
acggcacggtcagagacattctcaaagtctccacttggcctagtaatagcggag

SEQ ID NO: 659 - DNA, 54 bp, artificial sequence
  Synthetic
ccgccgtcatgatcccgccccggtaagtctgcacttggcctagtaatagtaagc

SEQ ID NO: 660 - DNA, 54 bp, artificial sequence
  Synthetic
ccgccgtcatgatcccgccccgtaactgaaccatccggccctagaacgagaagc

SEQ ID NO: 661 - DNA, 54 bp, artificial sequence
  Synthetic
ctggagatgcacccccaaccgagaagcctctctttcggcccgaattagagaatc

SEQ ID NO: 662 - DNA, 54 bp, artificial sequence
  Synthetic
aacgccccgggcatcaagtattgtagttaccctttcggcccgagaacgagtagc

SEQ ID NO: 663 - DNA, 54 bp, artificial sequence
  Synthetic
ccggagggctccgagacattcgcaacttacccattcggccctagaacgagaagc

SEQ ID NO: 664 - DNA, 54 bp, artificial sequence
  Synthetic
ctggagctgaacccccaaccgagaatactctcgtttggcctcgtaatagaaatc

SEQ ID NO: 665 - DNA, 54 bp, artificial sequence
  Synthetic
ccgggccggtccgagacattctcaaagtctgcacttggcctagtaatagtaagc

SEQ ID NO: 666 - DNA, 54 bp, artificial sequence
  Synthetic
ccggcacggtccgagacattctcaaagtctgcacttggcctagtaacgagtagc

SEQ ID NO: 667 - DNA, 54 bp, artificial sequence
  Synthetic
ccggcagggtgcgagacccccgtaacttaaccattcggccctagaacgagaagc

SEQ ID NO: 668 - DNA, 54 bp, artificial sequence
  Synthetic
ccgccgtcatgatcccgccccggtcgggactcatttcgcctactaacgagaatc

SEQ ID NO: 669 - DNA, 54 bp, artificial sequence
  Synthetic
ccggcccgtttccctcaacaggcaagcctccctttcggcccgaattagagaatc

SEQ ID NO: 670 - DNA, 54 bp, artificial sequence
  Synthetic
acgggcctacgaggtaagtcccgtctagacggagacgccgaaccatcgagaatc

SEQ ID NO: 671 - DNA, 54 bp, artificial sequence
  Synthetic
cccccccgtttaacccctgctgcctgcctccctttcggcccgaattagagaatc

SEQ ID NO: 672 - DNA, 54 bp, artificial sequence
  Synthetic
ccggcacggtccgagacattctcaaagtcttctttcggcccgaattagagaatc

SEQ ID NO: 673 - DNA, 54 bp, artificial sequence
  Synthetic
ccgccgtcaggatcacccccctcaaagtcagcacttggcctagtaatcgcaagc

SEQ ID NO: 674 - DNA, 54 bp, artificial sequence
  Synthetic
ccgtggagctcgattagcccctcctcttactaacttggcctaggttaaagaatc

SEQ ID NO: 675 - DNA, 54 bp, artificial sequence
  Synthetic
ctggagatgaacccgaatccgagcagtctcccacttggcctagtaatagggagc

SEQ ID NO: 676 - DNA, 54 bp, artificial sequence
  Synthetic
acggcacggtccgagtcattgtcaaagtctgtacttggcctagtaatgacggag

SEQ ID NO: 677 - DNA, 54 bp, artificial sequence
  Synthetic
cccccccgtttatcccgtcccgggcgttacccatacggccctagaacgagaagc

SEQ ID NO: 678 - DNA, 54 bp, artificial sequence
  Synthetic
ctggcacggcacccccaacacagaagcatctcttgcggcccactaactagatac

SEQ ID NO: 679 - DNA, 54 bp, artificial sequence
  Synthetic
ccggctcattccgagtcattctacagctctccacttgtcgtagtaataggaagc

SEQ ID NO: 680 - DNA, 54 bp, artificial sequence
  Synthetic
acggagatgtcccctcaacaggcaagcctccctttcggcccgaattcgagaagc

SEQ ID NO: 681 - DNA, 54 bp, artificial sequence
  Synthetic
cgacctcaatggccgaccagcgtaacttacccattcggccctagaacgagaagc

SEQ ID NO: 682 - DNA, 54 bp, artificial sequence
  Synthetic
ccgatgtcatgatctcaacaggcaagcctccctttcggcccgaattagagaagc

SEQ ID NO: 683 - DNA, 54 bp, artificial sequence
  Synthetic
ccggcacggtccgatcaacaggcaagcctccctttcggcccgaattagagaatc

SEQ ID NO: 684 - DNA, 54 bp, artificial sequence
  Synthetic
cgacctgaatggccgacctgccgccagtcagcacttggcctaggagtagcaagc

SEQ ID NO: 685 - DNA, 54 bp, artificial sequence
  Synthetic
ccggcacggtccgagacattctcaaggtctgcacttggccctagaacgtgaagc

SEQ ID NO: 686 - DNA, 54 bp, artificial sequence
  Synthetic
ccgccgtcatgacctcaacaggcaagcctccctttcggcccgaattagagaatc

SEQ ID NO: 687 - DNA, 54 bp, artificial sequence
  Synthetic
ccggcacggtccgagacccccgtaacttacccattcggccctagaacgagaagc

SEQ ID NO: 688 - DNA, 54 bp, artificial sequence
  Synthetic
cctgcacggtccgagacattctcaaagtctgcacttggcctagtaatagtaagc

SEQ ID NO: 689 - DNA, 54 bp, artificial sequence
  Synthetic
ccgccgtcatgatcccgcccgagaagcctctctttcggcccgaattagagaatc

SEQ ID NO: 690 - DNA, 54 bp, artificial sequence
  Synthetic
acggcacggtccgagacattctcaaactctgcccttggcctagtaatagcggag

SEQ ID NO: 691 - DNA, 54 bp, artificial sequence
  Synthetic
aacgccccgggcatcaagtacgctaagactgcacttggcctagtaatagcggag

SEQ ID NO: 692 - DNA, 54 bp, artificial sequence
  Synthetic
ccggctcggtcgaagaccccctcgggtctcgcacttggcctagtaatagtaagc

SEQ ID NO: 693 - DNA, 54 bp, artificial sequence
  Synthetic
ccgccgtcatgatcccgcccaggtcggtacccatttggccctagaacgagaacc

SEQ ID NO: 694 - DNA, 54 bp, artificial sequence
  Synthetic
ccggcccggtccgagaggttctcaaagtccgcacttggcctggtaatagcaatc

SEQ ID NO: 695 - DNA, 54 bp, artificial sequence
  Synthetic
ccggtgtcgggattactgcccggccgtgacggagtcgccgaatcaagtatcagc

SEQ ID NO: 696 - DNA, 54 bp, artificial sequence
  Synthetic
ccgatgtcatgatcttccccctcggcttacgcattcggccctagaacgagaagc

SEQ ID NO: 697 - DNA, 54 bp, artificial sequence
  Synthetic
aacgccccgggcatcaagtattgtagttacccttacgccgaaccaacgagaatc

SEQ ID NO: 698 - DNA, 54 bp, artificial sequence
  Synthetic
acggagatgtcccctccacaggcaagcctccctttcggcccgaattagagaatc

SEQ ID NO: 699 - DNA, 54 bp, artificial sequence
  Synthetic
ccggcagggtgcgagacattctcaaagtctgcacttaccgaatcaagtgtaatc

SEQ ID NO: 700 - DNA, 54 bp, artificial sequence
  Synthetic
ccggcacggtccgagacccccttccacaacccattcggcctagtaactagaagc

SEQ ID NO: 701 - DNA, 54 bp, artificial sequence
  Synthetic
ccggcacggtcctcacccggcgtccctctcccacttggcctagtaatagtaagc

SEQ ID NO: 702 - DNA, 54 bp, artificial sequence
  Synthetic
ccggcacggcacccccaaccgagaagcctctctttcgtcctaataataagacgc

SEQ ID NO: 703 - DNA, 54 bp, artificial sequence
  Synthetic
ccggagaggcggatgaccgaacctgcatttacattcgacgtagcaagtatcagc

SEQ ID NO: 704 - DNA, 54 bp, artificial sequence
  Synthetic
ccgggccggcacccccaaccgagaagcctctctttcggcccgaattagagaatc

SEQ ID NO: 705 - DNA, 54 bp, artificial sequence
  Synthetic
cccgcagaatgttctcaacaggcaagcctccctttcggcccgaattagagaatc

SEQ ID NO: 706 - DNA, 54 bp, artificial sequence
  Synthetic
ccggcacggtccgagacattctcaaagtaccctttcggcccgaattagagaatc

SEQ ID NO: 707 - DNA, 54 bp, artificial sequence
  Synthetic
ccgccgccatgatcccgccccgttcggtgctcattcagccctagcacgagaagc

SEQ ID NO: 708 - DNA, 54 bp, artificial sequence
  Synthetic
cgggagatgaacccccaaccgagaagcctctctttcggcctagtaatagcggag

SEQ ID NO: 709 - DNA, 54 bp, artificial sequence
  Synthetic
ccgccgtcatgatcccgccccggtcgggacgcacttggcctagtaatagcggag

SEQ ID NO: 710 - DNA, 54 bp, artificial sequence
  Synthetic
cccgaccgtttccctcaacaggcaagcctccctttcggcccgaattagagaatc

SEQ ID NO: 711 - DNA, 54 bp, artificial sequence
  Synthetic
ccgatgtcatgatcttgccccggtaggtctccacttgtcctagtaatagcggag

SEQ ID NO: 712 - DNA, 54 bp, artificial sequence
  Synthetic
cgggagatgaacccccaaccgagaagcctctctttcggcccgaatatgagaagc

SEQ ID NO: 713 - DNA, 54 bp, artificial sequence
  Synthetic
acgaagagatgatcccgccccgtaacttacccattcggccctagaacgagaagc

SEQ ID NO: 714 - DNA, 54 bp, artificial sequence
  Synthetic
ccggcgtcatgatcgcgtcccgggcgttacccatacggccctagaacgagaagc

SEQ ID NO: 715 - DNA, 54 bp, artificial sequence
  Synthetic
ccggcccggcacccccaagcgagaaccctctctttaggcccagtaatacggcgc

SEQ ID NO: 716 - DNA, 54 bp, artificial sequence
  Synthetic
cccggaagatcctcgtccgaagtaacgttcccattcggccctagatcgaatagc

SEQ ID NO: 717 - DNA, 54 bp, artificial sequence
  Synthetic
ccggtgtcgggattactgcccggccgtgatgcacttggcctagtaatagtaagc

SEQ ID NO: 718 - DNA, 54 bp, artificial sequence
  Synthetic
cccggcggatcatcgtccgcagtaacgctctctttcggaccgaggtctagaatc

SEQ ID NO: 719 - DNA, 54 bp, artificial sequence
  Synthetic
acggcacggtccgagacattctcaaagtctgcacttggcctagtaatagcggag

SEQ ID NO: 720 - DNA, 54 bp, artificial sequence
  Synthetic
ccgccgtcttgatcccgccccgcaacttaccaattcggcactagaacgagaagc

SEQ ID NO: 721 - DNA, 54 bp, artificial sequence
  Synthetic
ccggcacggtccgagaccccctcggctctcccacttggcctagcaatagtaagc

SEQ ID NO: 722 - DNA, 54 bp, artificial sequence
  Synthetic
cccggaagatcctcgtccgaagtaacgttcccattcggcctagtaatagcgatc

SEQ ID NO: 723 - DNA, 54 bp, artificial sequence
  Synthetic
ccgggaggactgatgaccccctcggcttacacacttggcctaggaactggacac

SEQ ID NO: 724 - DNA, 54 bp, artificial sequence
  Synthetic
ccggcacggcacccccaaccggtaacttacccattcggccctagaacgagaagc

SEQ ID NO: 725 - DNA, 54 bp, artificial sequence
  Synthetic
acgggccaatccccgctactggcaagcttccctttcggcccgaattagagaatc

SEQ ID NO: 726 - DNA, 54 bp, artificial sequence
  Synthetic
ccgccaaagtgcgagaccctcgtaacatacccattcggccctagaacgtcaagc

SEQ ID NO: 727 - DNA, 54 bp, artificial sequence
  Synthetic
ccgacacggtgatcccgccccggtcgggacggaatcgccgaatcaagtgtgcgc

SEQ ID NO: 728 - DNA, 54 bp, artificial sequence
  Synthetic
cccggaagatcctcgtccgaagtaacgttcccaatcgccgaatcaacgagaatc

SEQ ID NO: 729 - DNA, 54 bp, artificial sequence
  Synthetic
acggcacggtccgagacattctcaaagttccctttcggcccgaattagagaatc

SEQ ID NO: 730 - DNA, 54 bp, artificial sequence
  Synthetic
acgggccgatgcacccttactcacagcctctctttcggcccgaattagagaatc

SEQ ID NO: 731 - DNA, 54 bp, artificial sequence
  Synthetic
acgggacgttgcacccagtctctcccaatctcattcggccctagaacgagaagc

SEQ ID NO: 732 - DNA, 54 bp, artificial sequence
  Synthetic
ccgccgtcatgatcccgccccggtcggtacccatacggccctagaacgagacgc

SEQ ID NO: 733 - DNA, 54 bp, artificial sequence
  Synthetic
ccgccatcatgattacgccccggaaggttcccattcggcccaagttcaagaagc

SEQ ID NO: 734 - DNA, 54 bp, artificial sequence
  Synthetic
ccggcagggtgcgagacattctcaaagtctgcatttcgcctaccaacgagaatc

SEQ ID NO: 735 - DNA, 54 bp, artificial sequence
  Synthetic
ccgccgtcatgatcccgccccggtagcctccctttcggcccgaattagagaatc

SEQ ID NO: 736 - DNA, 54 bp, artificial sequence
  Synthetic
cccggaagatcctcgtccgaagtaacgttcccattcggccctagaatagcgatc

SEQ ID NO: 737 - DNA, 54 bp, artificial sequence
  Synthetic
ccggcagggtgcgagacccccgtaacttacccattcggccctagaacgagaagc

SEQ ID NO: 738 - DNA, 54 bp, artificial sequence
  Synthetic
cccggaagatcctcgtccgaagtaacgttcccattcggcccgaattagagaagc

SEQ ID NO: 739 - DNA, 54 bp, artificial sequence
  Synthetic
ccggagatgaacccccaatctagaatcctctcataccgcccgatatagttaagc

SEQ ID NO: 740 - DNA, 54 bp, artificial sequence
  Synthetic
ccggaacgctacccccgccccgcgtcttacgcacttgtgctagtaacagaccgc

SEQ ID NO: 741 - DNA, 54 bp, artificial sequence
  Synthetic
ccggcacggtcctcacccggaatccacatccctttccggttgagttcgataatc

SEQ ID NO: 742 - DNA, 54 bp, artificial sequence
  Synthetic
cccgcagaagcgtcgtccgaagtaacgttccctttcggcccgaattagagaatc

SEQ ID NO: 743 - DNA, 54 bp, artificial sequence
  Synthetic
ccgggagggtttcccggaccactcagcctcccacttggccaagtaatagggagc

SEQ ID NO: 744 - DNA, 54 bp, artificial sequence
  Synthetic
ccggcctggtcccctgaacaagcaagcctctctttcggcccgaattagagaatc

SEQ ID NO: 745 - DNA, 54 bp, artificial sequence
  Synthetic
ccggcgtcatgatcccgtcccgggccttacccatacggccctagaacgagaagc

SEQ ID NO: 746 - DNA, 54 bp, artificial sequence
  Synthetic
cgacctcaatggccgaccagccggcgttacccatacggccctagaacgagaagc

SEQ ID NO: 747 - DNA, 54 bp, artificial sequence
  Synthetic
ccgccgtcatgatccccattcgcaacttacccattcggccctagaacgagaagc

SEQ ID NO: 748 - DNA, 54 bp, artificial sequence
  Synthetic
ccggcacggcacccccaaccgagaagcctctctttcggcccgacaattgtaagc

SEQ ID NO: 749 - DNA, 54 bp, artificial sequence
  Synthetic
ccggcgagatgatcccgccccgtaacttacccattcggccctagaacgagaagc

SEQ ID NO: 750 - DNA, 54 bp, artificial sequence
  Synthetic
acgaggagctccgagacccccgtaacttacccattcggccctagaacgagaagc

SEQ ID NO: 751 - DNA, 54 bp, artificial sequence
  Synthetic
atggcacaacgagctcaacaggcaagcctccctttcggcccgaattagagaatc

SEQ ID NO: 752 - DNA, 54 bp, artificial sequence
  Synthetic
ccggcacagcaccccgcccccgtggcttacgcccttggcctagtattagaacgc

SEQ ID NO: 753 - DNA, 54 bp, artificial sequence
  Synthetic
ccggctcggcacccccaacagagacgtctctctttcggcccgaatttgagaatc

SEQ ID NO: 754 - DNA, 54 bp, artificial sequence
  Synthetic
ccgccgtggtccgagaccccctcaagcctccctttcggcccgaattagagaagc

SEQ ID NO: 755 - DNA, 54 bp, artificial sequence
  Synthetic
ccgccgtcatgatcccgccccgtaacttacccattcggccctagaacgacaagc

SEQ ID NO: 756 - DNA, 54 bp, artificial sequence
  Synthetic
cccggaagaacctcgtccgaagtaacggtcccaatcggccctagatcgagaagc

SEQ ID NO: 757 - DNA, 54 bp, artificial sequence
  Synthetic
ccggcacggtccgagacattctcaaagtctgcactcgccgaatcaacgagaacc

SEQ ID NO: 758 - DNA, 54 bp, artificial sequence
  Synthetic
cgacctcaatggccgaccagccgccagagcggaatcgcggaatcaacgagaacc

SEQ ID NO: 759 - DNA, 54 bp, artificial sequence
  Synthetic
cccggaagatcctccgccgtagcaagcctccctttcggcccgaattagagaatc

SEQ ID NO: 760 - DNA, 54 bp, artificial sequence
  Synthetic
ccggcacgggccgacacgttctccaagtctgcacttggcgtagtaatcaggagc

SEQ ID NO: 761 - DNA, 54 bp, artificial sequence
  Synthetic
acgaaggtgctgatgaccccctcggcgtacgcattcggccctagaacgagaagc

SEQ ID NO: 762 - DNA, 54 bp, artificial sequence
  Synthetic
ctggagatgaacccccccccctcggcttacgcacttggcctagtaacgagtaac

SEQ ID NO: 763 - DNA, 54 bp, artificial sequence
  Synthetic
ccggcacggtccgagaccccctcggctctcccacttggcccagtaatagtaagc

SEQ ID NO: 764 - DNA, 54 bp, artificial sequence
  Synthetic
cccggatggtcccctcaacaggcaagcctccctttcggcccgaattagagaatc

SEQ ID NO: 765 - DNA, 54 bp, artificial sequence
  Synthetic
ccggcgtaatgatcccgccccgggcgttacccatacggacctagaacgataagc

SEQ ID NO: 766 - DNA, 54 bp, artificial sequence
  Synthetic
ccgccgtcatgatcccgccccgctaagactgcacttggcctagtaatagcggag

SEQ ID NO: 767 - DNA, 54 bp, artificial sequence
  Synthetic
ccggcaccatgatcccgccccggtcgggacggaatcgccgaatcaacgagaatc

SEQ ID NO: 768 - DNA, 54 bp, artificial sequence
  Synthetic
acggcacggtccgagacattctcaaagtctgcacttggcctagtatcgagaatc

SEQ ID NO: 769 - DNA, 54 bp, artificial sequence
  Synthetic
acggcactacgaggtaagtcccgtctagacggagacgccgaaccatcgagaatc

SEQ ID NO: 770 - DNA, 54 bp, artificial sequence
  Synthetic
cgggcacggcaccccgtatcgataagcctctctttcggcgcagtaactaaacac

SEQ ID NO: 771 - DNA, 54 bp, artificial sequence
  Synthetic
ccggcaatgtccgcccgtcccgggcgttacccatacggccctagaacgagaagc

SEQ ID NO: 772 - DNA, 54 bp, artificial sequence
  Synthetic
ccgccgtcatgatcccgccccggtcgggacggaatcgccgaatcaacgataagc

SEQ ID NO: 773 - DNA, 54 bp, artificial sequence
  Synthetic
ctggcgtcacgatcccgtcccgggcgttactcatacggccctagaacgagaagc

SEQ ID NO: 774 - DNA, 54 bp, artificial sequence
  Synthetic
acgccgtcatgatcccgccccggtcgggacggaatcgccgaatcaacgagaatc

SEQ ID NO: 775 - DNA, 54 bp, artificial sequence
  Synthetic
cccccccgtttaacccctgctgtctgcgtcccacttggcctagtaacgtgaaac

SEQ ID NO: 776 - DNA, 54 bp, artificial sequence
  Synthetic
cccgcacggtccgagacattctcaaagtctgcacttggcctagtaatagcggag

SEQ ID NO: 777 - DNA, 54 bp, artificial sequence
  Synthetic
cccggaagatcctcgtccgaagtaacgttcccattcggcctgagatcgataatc

SEQ ID NO: 778 - DNA, 54 bp, artificial sequence
  Synthetic
ccggcacggtccgagacattctcaacgttccctttcggcccgaattagagaatc

SEQ ID NO: 779 - DNA, 54 bp, artificial sequence
  Synthetic
ccgtggagatgatcccgccccgtaacttacccattcggccctatagccactagc

SEQ ID NO: 780 - DNA, 54 bp, artificial sequence
  Synthetic
acgaagagctccgagacattcgcaacttacccattcggccctagaacgagaagc

SEQ ID NO: 781 - DNA, 54 bp, artificial sequence
  Synthetic
ccggcacggtgggagacactctcaaagtctgcacttggcgtaagaacgtgaagc

SEQ ID NO: 782 - DNA, 54 bp, artificial sequence
  Synthetic
ccggcacgctccgaggcaagccgccgtgacccattcggcctagtaactagaagc

SEQ ID NO: 783 - DNA, 54 bp, artificial sequence
  Synthetic
cgacctcaatggccgaccagccgccgtgacccacttagcctactaacgcgaatc

SEQ ID NO: 784 - DNA, 54 bp, artificial sequence
  Synthetic
ccgccgtcatgatcccgccccgtaacttacggaatcgccgaatcaacgagaacc

SEQ ID NO: 785 - DNA, 54 bp, artificial sequence
  Synthetic
ccggcacggcacccccaaccgagaacttacccattcggccctagaacgagaagc

SEQ ID NO: 786 - DNA, 54 bp, artificial sequence
  Synthetic
ccggcacggcacccccaaccgagatgcctatctttcggcccgaatccgagaatc

SEQ ID NO: 787 - DNA, 54 bp, artificial sequence
  Synthetic
acggagatgtccccccaaccgagaagcctctctttcggcccgaattagagaatc

SEQ ID NO: 788 - DNA, 54 bp, artificial sequence
  Synthetic
ccggcgcggctgatgacccccctcccaaccgcacttgcccgaataatagcaagc

SEQ ID NO: 789 - DNA, 54 bp, artificial sequence
  Synthetic
ccgccgtcaagatcccgccccggtcgcgactgaatcgccgaatcaagtagaacc

SEQ ID NO: 790 - DNA, 54 bp, artificial sequence
  Synthetic
acgggccgatcccctcaacaggcaagttaccctttcggcccgaattagagaatc

SEQ ID NO: 791 - DNA, 54 bp, artificial sequence
  Synthetic
ccggcacggcatccccaaccgagaagcttctctatcggcccgaaatagagaatc

SEQ ID NO: 792 - DNA, 54 bp, artificial sequence
  Synthetic
ccgggacggtccgagacattctcaaagtcggtacttggcctaagcacgagtagc

SEQ ID NO: 793 - DNA, 54 bp, artificial sequence
  Synthetic
acggagcggtccgagacattctcaaagtccccattcggccctagaacgagaagc

SEQ ID NO: 794 - DNA, 54 bp, artificial sequence
  Synthetic
ccgggacggtccgagactttctcaatggctgcacttggcctagtaatagtaagc

SEQ ID NO: 795 - DNA, 54 bp, artificial sequence
  Synthetic
acgggacgttgcacccagtctgtaacttacccattcggccctagaacgagaagc

SEQ ID NO: 796 - DNA, 54 bp, artificial sequence
  Synthetic
acgggccgatgcactcaacaggcaagcctccctttcggcccgaattagagaagc

SEQ ID NO: 797 - DNA, 54 bp, artificial sequence
  Synthetic
ccgatgtcgggattactgcccggccgtgacggagtcgtcggaccaagtctaagc

SEQ ID NO: 798 - DNA, 54 bp, artificial sequence
  Synthetic
ccgggtacctccgagacattcgccgcttacgcactgggcctagtaactagaatc

SEQ ID NO: 799 - DNA, 54 bp, artificial sequence
  Synthetic
ccgggcagctccgagacattcgttaatactgcacttagcctagtaatagcggag

SEQ ID NO: 800 - DNA, 54 bp, artificial sequence
  Synthetic
cgacctcaatggccgaccagccgccagagcggaatcgccgaatcaacgataagc

SEQ ID NO: 801 - DNA, 54 bp, artificial sequence
  Synthetic
ccggcacggcacccgacccccgtaacttacccattcggccctagaacgagaagc

SEQ ID NO: 802 - DNA, 54 bp, artificial sequence
  Synthetic
acgagtgctcagtgcgctttatgctcactcccgttcggcccgagatcgagaatc

SEQ ID NO: 803 - DNA, 54 bp, artificial sequence
  Synthetic
ccggcacggcaccagacccccgtaacttacccattcggccctagaacgagaagc

SEQ ID NO: 804 - DNA, 54 bp, artificial sequence
  Synthetic
ccggcacggtctgagacattctcaaagtctgcacttggcctagtaatagtggag

SEQ ID NO: 805 - DNA, 54 bp, artificial sequence
  Synthetic
ccggcacgctccgagacattcgcaacttacccattcggccctagaacgagaagc

SEQ ID NO: 806 - DNA, 54 bp, artificial sequence
  Synthetic
cgacctcaatggccgaccagacgccagagcggaatcgccgaatcaacgagaacc

SEQ ID NO: 807 - DNA, 54 bp, artificial sequence
  Synthetic
acgaagggactgataaccccctcggcttacgcagacgccgaaccaagggtaagc

SEQ ID NO: 808 - DNA, 54 bp, artificial sequence
  Synthetic
ccggcatgacacacccaaccgagaagcctctctttcggcccgaattagagaatc

SEQ ID NO: 809 - DNA, 54 bp, artificial sequence
  Synthetic
cgggagatgaacccccaaccgagaagcctctctttcggcccgaattagagaagc

SEQ ID NO: 810 - DNA, 54 bp, artificial sequence
  Synthetic
cccgggtgctccgagagcccagtggctctcgcatttggccttggaataggaagc

SEQ ID NO: 811 - DNA, 54 bp, artificial sequence
  Synthetic
cccggaagatcctccgccgtagtaacataagcacttagcccagtaatagcggag

SEQ ID NO: 812 - DNA, 54 bp, artificial sequence
  Synthetic
ccggcgtcatgatcccgtcccgggcggtaccaatacggccctagaacgagaagc

SEQ ID NO: 813 - DNA, 54 bp, artificial sequence
  Synthetic
ccgggaagatcgtagtccgaggtagcttacccattcggccgtagaacgagaggc

SEQ ID NO: 814 - DNA, 54 bp, artificial sequence
  Synthetic
cgacctcaatggccgaccagccgccatcacccattccgccctagatcgagaagc

SEQ ID NO: 815 - DNA, 54 bp, artificial sequence
  Synthetic
ccgccgtcatgatcccggcccggtcgggacggaatcgccgaatcaagtgtaatc

SEQ ID NO: 816 - DNA, 54 bp, artificial sequence
  Synthetic
ccggcacggctcagggtactctttacagacggagatgccgaaccaattgtaagc

SEQ ID NO: 817 - DNA, 54 bp, artificial sequence
  Synthetic
ccggcacggtccgagacattctcaaagagcggaatcgccgaatcaacgagaacc

SEQ ID NO: 818 - DNA, 54 bp, artificial sequence
  Synthetic
cccgtgagggccgtgagatcagctaagatcccattcggccctacaacgagaagc

SEQ ID NO: 819 - DNA, 54 bp, artificial sequence
  Synthetic
ccggcacggtccgagacattctcaaagtctgcacttggcctagtaatagcaatc

SEQ ID NO: 820 - DNA, 54 bp, artificial sequence
  Synthetic
ccggcccggtccgagacattctcaaactctgcacttggccgaggaataaccagc

SEQ ID NO: 821 - DNA, 54 bp, artificial sequence
  Synthetic
cgacctcaatggccgaccagcggccgtggcccattcggcctagtaactagaagc

SEQ ID NO: 822 - DNA, 54 bp, artificial sequence
  Synthetic
ccggcccggtccgagacattctcaaactctgcacttggcctagtaatagtaagc

SEQ ID NO: 823 - DNA, 54 bp, artificial sequence
  Synthetic
actgcgcgattcgggacattctcaaagtcagcacttggcctaggaataccggag

SEQ ID NO: 824 - DNA, 54 bp, artificial sequence
  Synthetic
cgacctcaatggccgaccagccgccagagcggaatcgccgaatcaacgagaacc

SEQ ID NO: 825 - DNA, 54 bp, artificial sequence
  Synthetic
cccgcccgtttccctcaacaggcaagcctccctttcggcccgaattcgagaagc

SEQ ID NO: 826 - DNA, 54 bp, artificial sequence
  Synthetic
cgacctcaatggccgaccagccgccgtgccccattcggcctagtagctagaagc

SEQ ID NO: 827 - DNA, 54 bp, artificial sequence
  Synthetic
accaaagggctcatgaccccctcggctttcgcacatggcctactaatatccagc

SEQ ID NO: 828 - DNA, 54 bp, artificial sequence
  Synthetic
ccgccgtcatgatcccgccccgtaacttgcgcattctgcccgagaacgtgaagc

SEQ ID NO: 829 - DNA, 54 bp, artificial sequence
  Synthetic
ccgcggtcaggatctcccccctcacagtctgcacttggcctaggtatagcaagc

SEQ ID NO: 830 - DNA, 54 bp, artificial sequence
  Synthetic
ctgtcacggtccgagacattctcaaagtcacgacttggcctagcaatagtaagc

SEQ ID NO: 831 - DNA, 54 bp, artificial sequence
  Synthetic
ccgccgtcatgatcccgccccggtcggtacacatttggcccgagaacgaggagc

SEQ ID NO: 832 - DNA, 54 bp, artificial sequence
  Synthetic
cccggaagatcctcccccggcgtccacctcccattcggcctgagatcgataatc

SEQ ID NO: 833 - DNA, 54 bp, artificial sequence
  Synthetic
cccggaagatcctcgtcccaagtaagcctccctttcggcccgaattagagaagc

SEQ ID NO: 834 - DNA, 54 bp, artificial sequence
  Synthetic
ccggcacggtccgagactgtctcaaagtctccacttggcctcgtaatagcggag

SEQ ID NO: 835 - DNA, 54 bp, artificial sequence
  Synthetic
ccggcacggcacccccaacccgctaagatcccattcggccctagaacgagaagc

SEQ ID NO: 836 - DNA, 54 bp, artificial sequence
  Synthetic
acgggacgttgcacgacattcgctaagatcccattcggccctagaacgagaagc

SEQ ID NO: 837 - DNA, 54 bp, artificial sequence
  Synthetic
ccgccgtcatgatcccgccccggtcggtacccattcggccctagaacgagaatc

SEQ ID NO: 838 - DNA, 54 bp, artificial sequence
  Synthetic
cgacctcaatggccgaccagccgccgtgacccattcggcccagtacctagaaac

SEQ ID NO: 839 - DNA, 54 bp, artificial sequence
  Synthetic
cgggcgtcatgatcccgtcccgggcgttacccagacggacctagaacgagaagc

SEQ ID NO: 840 - DNA, 54 bp, artificial sequence
  Synthetic
ccggcatggtcccctcaacaggcaagcctccctttcggcccgaattagagaagc

SEQ ID NO: 841 - DNA, 54 bp, artificial sequence
  Synthetic
acggagcggctgatgaccccctcgctttacgcattcggccatagtacgagaaac

SEQ ID NO: 842 - DNA, 54 bp, artificial sequence
  Synthetic
ccgccgtcatgatcccgccccgtaacttgcccattcggcgcaagaacgagaagc

SEQ ID NO: 843 - DNA, 54 bp, artificial sequence
  Synthetic
cgaaggaccgcttcacccggccgccagagcggaatcgccgaatcaacgagaacc

SEQ ID NO: 844 - DNA, 54 bp, artificial sequence
  Synthetic
cccgcagaagcgtcgtcctaagtaacgttcccattcggcactagatcgagaagc

SEQ ID NO: 845 - DNA, 54 bp, artificial sequence
  Synthetic
ccggcacggtccgagacattcgctaagactgcacttggcctagtaatagcggag

SEQ ID NO: 846 - DNA, 54 bp, artificial sequence
  Synthetic
ccggcacggttcgagacattctcaaagtctgcacttggcctagtaatagcgatc

SEQ ID NO: 847 - DNA, 54 bp, artificial sequence
  Synthetic
ccgccgtcatgatcccgccccggtcgggacggaatcgccgaaccaacgagaatc

SEQ ID NO: 848 - DNA, 54 bp, artificial sequence
  Synthetic
cgggagatgaacccccaaccgagaagcctctctttcggcgagccaagtctaagc

SEQ ID NO: 849 - DNA, 54 bp, artificial sequence
  Synthetic
ccggcacgctccgagacccccgtaacttacccattcggccctagaacgagaagc

SEQ ID NO: 850 - DNA, 54 bp, artificial sequence
  Synthetic
cccggaagatcatcccgccccggtcgggacggaatcgccgaatcaacgagaatc

SEQ ID NO: 851 - DNA, 54 bp, artificial sequence
  Synthetic
cgggagatgaacccccaaccgagaagcctctcattccgccctataatagcaagc

SEQ ID NO: 852 - DNA, 54 bp, artificial sequence
  Synthetic
ccggcacggcacccccaattcgcaacttacccattcggccctagaacgagaagc

SEQ ID NO: 853 - DNA, 54 bp, artificial sequence
  Synthetic
acggcacggtgcgagacattctcaaagtctgcacttggcctagtaataggaacc

SEQ ID NO: 854 - DNA, 54 bp, artificial sequence
  Synthetic
ccggcacggtcggagaccccctcggctctctcacttggcctggtaacagtaagc

SEQ ID NO: 855 - DNA, 54 bp, artificial sequence
  Synthetic
ccgccgtcatgaccccgccccggtcgggacggaatcgccgaatcaagtgtaatc

SEQ ID NO: 856 - DNA, 54 bp, artificial sequence
  Synthetic
ccggcacgctccgagacatactcaaagtacccattcggccctagaacgagaagc

SEQ ID NO: 857 - DNA, 54 bp, artificial sequence
  Synthetic
ccggcacggtccgagaccccctcgtgtctcccattcggcccgagctagagaaac

SEQ ID NO: 858 - DNA, 54 bp, artificial sequence
  Synthetic
caggagatgaacctcccccccggaaagtctgcacttggcctagtaactaggatc

SEQ ID NO: 859 - DNA, 54 bp, artificial sequence
  Synthetic
cgacctcaatggccgaccagccgcaagactgcacttggcctagtaatagcaatc

SEQ ID NO: 860 - DNA, 54 bp, artificial sequence
  Synthetic
ccggagagctccgagacattcgctaagactgcacttggactagtaatagcggag

SEQ ID NO: 861 - DNA, 54 bp, artificial sequence
  Synthetic
ccggcacggcaccgcccctccccgggttacgtacttgtcgcagtaattaaacgc

SEQ ID NO: 862 - DNA, 54 bp, artificial sequence
  Synthetic
cccggaagatcctcgtccgaagtaacgttcccattcgcccctagatcgagaagc

SEQ ID NO: 863 - DNA, 54 bp, artificial sequence
  Synthetic
ccgccgtcatgatcccgccccggtcgggacggaatcggccaatcaacgagaacc

SEQ ID NO: 864 - DNA, 54 bp, artificial sequence
  Synthetic
ccggcacggtccgagacattctcaaagtctgcacttggcctaggattagcggag

SEQ ID NO: 865 - DNA, 54 bp, artificial sequence
  Synthetic
ccggcacggtccgagacacaggcaagcctccctttcggcccgaattagagaatc

SEQ ID NO: 866 - DNA, 54 bp, artificial sequence
  Synthetic
acggagatgtcccctcacagccgccagagcggaatcgccgaatcaacgagaacc

SEQ ID NO: 867 - DNA, 54 bp, artificial sequence
  Synthetic
ccggcagcgtacgagacacccgtaacttacccattcggccctagaacgagaagc

SEQ ID NO: 868 - DNA, 54 bp, artificial sequence
  Synthetic
ccggcagggtgcgaaacccccgtaacttacgcattcggtcctggtacgagaagc

SEQ ID NO: 869 - DNA, 54 bp, artificial sequence
  Synthetic
ccgatgagctccgagacattcgctaagatcccattcggccctagaacgagaagc

SEQ ID NO: 870 - DNA, 54 bp, artificial sequence
  Synthetic
ccggcagactgctagacccccgtaacttacccagtcggccgtagaacgagaagc

SEQ ID NO: 871 - DNA, 54 bp, artificial sequence
  Synthetic
ccgccgtcatgatcccgccccggtcgggacggaatcggcctgagttcgagaatc

SEQ ID NO: 872 - DNA, 54 bp, artificial sequence
  Synthetic
acgaaggtgccgatgacaccctctgctttctcactcggccctagaacgacaagc

SEQ ID NO: 873 - DNA, 54 bp, artificial sequence
  Synthetic
ccggcacggtccgagacattctcaaagtctgtacgtggcctagtaatcagaagc

SEQ ID NO: 874 - DNA, 54 bp, artificial sequence
  Synthetic
ccggcacggcatccccgctcgagacactgctctttcggcctagtaacacgaagc

SEQ ID NO: 875 - DNA, 54 bp, artificial sequence
  Synthetic
cccgcccgttgccctcaacaggcaagcctcactttcggcccgaattagagaatc

SEQ ID NO: 876 - DNA, 54 bp, artificial sequence
  Synthetic
ccggcacggctaatgaccccctcgccttacgcactcggcctactaatagaagac

SEQ ID NO: 877 - DNA, 54 bp, artificial sequence
  Synthetic
acggagaggcagatgacccacagtgcctattcattacgcctactaacgagcatc

SEQ ID NO: 878 - DNA, 54 bp, artificial sequence
  Synthetic
cccggaagatcctcgtcccacactgcctattcatttcgcctactaacgagaatc

SEQ ID NO: 879 - DNA, 54 bp, artificial sequence
  Synthetic
ccggtatgctccgagacatccgcaacttacacattcggccctagaacgagaggc

SEQ ID NO: 880 - DNA, 54 bp, artificial sequence
  Synthetic
cgaccacaatggccgagcagacgccgtgacccattcggccctatatcgagaagc

SEQ ID NO: 881 - DNA, 54 bp, artificial sequence
  Synthetic
cccgcagaagaggattaagagggccgcctccctttcggcccgaattagagaatc

SEQ ID NO: 882 - DNA, 54 bp, artificial sequence
  Synthetic
ccggcacggctgatgtccccctcgccagtcgcactaggccaagtaactcgaagc

SEQ ID NO: 883 - DNA, 54 bp, artificial sequence
  Synthetic
cccgccagctccgagacattcgctaagactgcacttggcctagtaatagcggag

SEQ ID NO: 884 - DNA, 54 bp, artificial sequence
  Synthetic
ccgccgtcatgatcccgccccggtcgggacggaatcggccctagatcgagaagc

SEQ ID NO: 885 - DNA, 54 bp, artificial sequence
  Synthetic
cgggagagctccgagacccccgtaacttacccattcggccctagaacgagaagc

SEQ ID NO: 886 - DNA, 54 bp, artificial sequence
  Synthetic
cccgccccgggcatcaagtattgtagttacccactcggcccgagaacgagtagc

SEQ ID NO: 887 - DNA, 54 bp, artificial sequence
  Synthetic
ccggcacggtccgagacattctcaaagtctgcacttggcctaggttaggcaagc

SEQ ID NO: 888 - DNA, 54 bp, artificial sequence
  Synthetic
ccggcacggtccgagaccccctcgacttacccattcggccctagaacgagaagc

SEQ ID NO: 889 - DNA, 54 bp, artificial sequence
  Synthetic
ccggcacggtccgagactcgctcggctctcccacttggcctagtaatagtaagc

SEQ ID NO: 890 - DNA, 54 bp, artificial sequence
  Synthetic
ccgccgtcaggattactccccggccggctcccgttcggcccgagatcgagaatc

SEQ ID NO: 891 - DNA, 54 bp, artificial sequence
  Synthetic
acgaggagctccgagacattcgctaagatcccattcggccctagaacgagaagc

SEQ ID NO: 892 - DNA, 54 bp, artificial sequence
  Synthetic
cccggaagatcctccgccgtagtaacataagcactcgtcccgagatcgagaagc

SEQ ID NO: 893 - DNA, 54 bp, artificial sequence
  Synthetic
ccggcagggtgcgagacattcccaaagtctgcacttacactaggaatagcggag

SEQ ID NO: 894 - DNA, 54 bp, artificial sequence
  Synthetic
ccggcacggtccgagacattctaaaagactgcactgggcctaggaaacgcaagc

SEQ ID NO: 895 - DNA, 54 bp, artificial sequence
  Synthetic
ccggcagggtgccctcaacaggcaagcctccctttcggcccgaattagagaatc

SEQ ID NO: 896 - DNA, 54 bp, artificial sequence
  Synthetic
ccggcacggtccgagacattctcaagcctccctttcggcccgaattagagaagc

SEQ ID NO: 897 - DNA, 54 bp, artificial sequence
  Synthetic
ccggcacaatgttctcaacaggcaagcctccctttcggcccgaattagagaatc

SEQ ID NO: 898 - DNA, 54 bp, artificial sequence
  Synthetic
ccggcgcggtccaagatttcctcggccctaccacttggcctagtaatagttagc

SEQ ID NO: 899 - DNA, 54 bp, artificial sequence
  Synthetic
cccgggagctccgagaccctctcggctttcgcacttgtcctgataatagcgagc

SEQ ID NO: 900 - DNA, 54 bp, artificial sequence
  Synthetic
cccggaagatcctccgcagagggccgtgacggagacgccgaaccatcgagaatc

SEQ ID NO: 901 - DNA, 54 bp, artificial sequence
  Synthetic
ccgccgtcatgatcccgccgcggttgggacggaatcgccgcatcaacgagaacc

SEQ ID NO: 902 - DNA, 54 bp, artificial sequence
  Synthetic
ccggcccggtccgagacattctcaaactctgcacgtggcctagtaatagcaagc

SEQ ID NO: 903 - DNA, 54 bp, artificial sequence
  Synthetic
ccgccgtcatgatcccgccccgtaacttacccattcggccctagaacgagaatc

SEQ ID NO: 904 - DNA, 54 bp, artificial sequence
  Synthetic
cctgggagctccgagaccctcgggccttacacactcggcctatccacgataaac

SEQ ID NO: 905 - DNA, 54 bp, artificial sequence
  Synthetic
ccggcacggtccgagacaccgagaagcctctctttcggcccgaattagagaatc

SEQ ID NO: 906 - DNA, 54 bp, artificial sequence
  Synthetic
ccgccgtcatcatcccgacccggtcgggacggaatcgccgaatcaagtgtaatc

SEQ ID NO: 907 - DNA, 54 bp, artificial sequence
  Synthetic
ccggagtcttgatcccgtcccgggcgttacgcatacggatctacaacgagaagc

SEQ ID NO: 908 - DNA, 54 bp, artificial sequence
  Synthetic
ccggcgtcatgatcccgtcccgggcggtacccatgcggccctagaacgagaagc

SEQ ID NO: 909 - DNA, 54 bp, artificial sequence
  Synthetic
ggactcgccacagaccgccccgtaacgttcccattcggcccgaattagagaatc

SEQ ID NO: 910 - DNA, 54 bp, artificial sequence
  Synthetic
cgggagatgaacccccaacccgtaacttacccattcggccctagaacgagaagc

SEQ ID NO: 911 - DNA, 54 bp, artificial sequence
  Synthetic
cgacctcaagggccgatcagccgcaagagcggaatggccgaatctacgagaacc

SEQ ID NO: 912 - DNA, 54 bp, artificial sequence
  Synthetic
ccggcacggtccaagaccccctcaagcctccctttcggcccgaattagagaagc

SEQ ID NO: 913 - DNA, 54 bp, artificial sequence
  Synthetic
ccggcagggtgcgagacccccgtaacttacccattcggcgaatcaagtattagc

SEQ ID NO: 914 - DNA, 54 bp, artificial sequence
  Synthetic
ccggcacggtccgtgacattctcaaagtctgcacttggcctagtaatagtaagc

SEQ ID NO: 915 - DNA, 54 bp, artificial sequence
  Synthetic
ccgccgtcatgatcccgccccgttcgggacggaatcgccgaatcaacgagaatc

SEQ ID NO: 916 - DNA, 54 bp, artificial sequence
  Synthetic
ccggcacggcacccccaacccgtaacttacccattcggccctagaacgagaagc

SEQ ID NO: 917 - DNA, 54 bp, artificial sequence
  Synthetic
cccgcagaagaggattaagagggccgtgtcccacttggcctagtaatagcggag

SEQ ID NO: 918 - DNA, 54 bp, artificial sequence
  Synthetic
ccggcacggtccgagaagtattgtagttacccactcggcccgagaacgagtagc

SEQ ID NO: 919 - DNA, 54 bp, artificial sequence
  Synthetic
ccgccgtcctccgagacattcgctaagactgcacttggcctagtaatagcggag

SEQ ID NO: 920 - DNA, 54 bp, artificial sequence
  Synthetic
ccggcacggtccgagacattctcaaagtctgcacttggcctacgaacgagtagc

SEQ ID NO: 921 - DNA, 54 bp, artificial sequence
  Synthetic
ccggtgtcgggattactgcccggccgtgacggagtcgccgaacattagagaatc

SEQ ID NO: 922 - DNA, 54 bp, artificial sequence
  Synthetic
acgggccgatgatcccgcccgagaagcctctctttcggcccgaattagagaatc

SEQ ID NO: 923 - DNA, 54 bp, artificial sequence
  Synthetic
ccggcacggtccgagatcctcagctccgacggagacgccgaaccatcgagaatc

SEQ ID NO: 924 - DNA, 54 bp, artificial sequence
  Synthetic
ccgccgtcatgatcccgccccgtaacttacccattcggccctagttcgataatc

SEQ ID NO: 925 - DNA, 54 bp, artificial sequence
  Synthetic
ccggcacggtccgagacattctcaaagtctgcacttggcctagtaatctgaagc

SEQ ID NO: 926 - DNA, 54 bp, artificial sequence
  Synthetic
ccggcacgatccgagacattctcaaagtctgcacttggcctattaacgagaatc

SEQ ID NO: 927 - DNA, 54 bp, artificial sequence
  Synthetic
ccgggcagctccgagacattcgctaagactgctcttggcctagcaatagcggag

SEQ ID NO: 928 - DNA, 54 bp, artificial sequence
  Synthetic
ccggcacggtgcatcaagtattgtagttacccactcggcccgagaacgagtagc

SEQ ID NO: 929 - DNA, 54 bp, artificial sequence
  Synthetic
ccgagaaacgccgagacccctccggcttacgcacttgccctagtaatcgatcac

SEQ ID NO: 930 - DNA, 54 bp, artificial sequence
  Synthetic
cccgcagaacagtggtacggtcgttaactcccattcggccgtagatcgagaaac

SEQ ID NO: 931 - DNA, 54 bp, artificial sequence
  Synthetic
ccgccggcatgatcccgccccggtcgggacggaatcgccgaatcaacgagaatc

SEQ ID NO: 932 - DNA, 54 bp, artificial sequence
  Synthetic
ccggcacggtccgagacaccctcaagcctccctttcggcccgaattagagaagc

SEQ ID NO: 933 - DNA, 54 bp, artificial sequence
  Synthetic
cccggaagatcctccgcctattgtagttaccctttcggcccgaattagagaatc

SEQ ID NO: 934 - DNA, 54 bp, artificial sequence
  Synthetic
ggactcgccacagaccgccccgcaagcctccctttcggcccgaattagagaatc

SEQ ID NO: 935 - DNA, 54 bp, artificial sequence
  Synthetic
ccgccgtcatgatccccattctcaaagtctgcacttggcctagtaatagcaatc

SEQ ID NO: 936 - DNA, 54 bp, artificial sequence
  Synthetic
ccggtcgggctgatgaccccctggactttcgcagtcgtcctatcagtagcagac

SEQ ID NO: 937 - DNA, 54 bp, artificial sequence
  Synthetic
ccggcacggcaccccaccccctcggcttacgcattcggccctagaacgagaagc

SEQ ID NO: 938 - DNA, 54 bp, artificial sequence
  Synthetic
ccggcagggtgcgagacccccgtaacttaccgattcggccctagaacgagaagc

SEQ ID NO: 939 - DNA, 54 bp, artificial sequence
  Synthetic
acggagagctccgagacattcgctaagactgcacttggcctagtaatagcaatc

SEQ ID NO: 940 - DNA, 54 bp, artificial sequence
  Synthetic
cggcagatgaacccccaaccgagaagcctctcatgccgccctagttatataagc

SEQ ID NO: 941 - DNA, 54 bp, artificial sequence
  Synthetic
accgccccgggcatcaagtattgtagttacccactcggcccgagaacgagtagc

SEQ ID NO: 942 - DNA, 54 bp, artificial sequence
  Synthetic
acgaaggcgctgatgaccccctcggcttacgcatacgccgaaccatagggaagc

SEQ ID NO: 943 - DNA, 54 bp, artificial sequence
  Synthetic
cgcggaagatcctcgtccgaagtaacgttcccattcggccctacatcgagaagc

SEQ ID NO: 944 - DNA, 54 bp, artificial sequence
  Synthetic
aacgccccgggcatcaagtattgtagttacccactcggcccgagaacgagaatc

SEQ ID NO: 945 - DNA, 54 bp, artificial sequence
  Synthetic
ccggcaccgaccgagaccctctcaaagtctgcacttggcctagtaacagccatc

SEQ ID NO: 946 - DNA, 54 bp, artificial sequence
  Synthetic
cgggagagctccgagacattcgctaagatcccattcggccctagaacgagaagc

SEQ ID NO: 947 - DNA, 54 bp, artificial sequence
  Synthetic
ccggtaagatggccgaccagccgccagagcggaatcgccgaatcaacgagaacc

SEQ ID NO: 948 - DNA, 54 bp, artificial sequence
  Synthetic
ccggagcggcacacccaaccgagaagcctctctctcggcgcgaattatcgaatc

SEQ ID NO: 949 - DNA, 54 bp, artificial sequence
  Synthetic
ccggcacggcgatcccgccccgtaacttacccattcggccctagaacgagaagc

SEQ ID NO: 950 - DNA, 54 bp, artificial sequence
  Synthetic
ccggcaaggtccgagacattctcaatgtctgcacttggccgtagaacgagaagc

SEQ ID NO: 951 - DNA, 54 bp, artificial sequence
  Synthetic
ccggcagggtgcgagacattctcaaggtctccacttgtcctagtaatagcggag

SEQ ID NO: 952 - DNA, 54 bp, artificial sequence
  Synthetic
ccgggagcggtttcctcccccccggcttacgcacttggtctagtaatagccaac

SEQ ID NO: 953 - DNA, 54 bp, artificial sequence
  Synthetic
cgagctcaatggccgaccagccgccgtaatccattcggacgtagatcgagaagc

SEQ ID NO: 954 - DNA, 54 bp, artificial sequence
  Synthetic
ccggcacggtccgagacattctcaaagtctgcacttggcccgagatcgagaatc

SEQ ID NO: 955 - DNA, 54 bp, artificial sequence
  Synthetic
ccgccgtcatgatcccgccccgtaacttacccattcggcctagtaactagaagc

SEQ ID NO: 956 - DNA, 54 bp, artificial sequence
  Synthetic
ccgccgtcatgatcccgccccggtcgggacggaatcggcccgagaacgagtagc

SEQ ID NO: 957 - DNA, 54 bp, artificial sequence
  Synthetic
ccggcacggtccgagacccccgggcgttacccatacggccctagaacgagaagc

SEQ ID NO: 958 - DNA, 54 bp, artificial sequence
  Synthetic
ccggcacggtcctcaaccggcgcatatatacctgttggtctaagttcgataacc

SEQ ID NO: 959 - DNA, 54 bp, artificial sequence
  Synthetic
ccgccgtcatgatcccccggcgtcccttacgcattcggccctagaacgagaagc

SEQ ID NO: 960 - DNA, 54 bp, artificial sequence
  Synthetic
ccggaacggtccgagacattctcaaagtctgcacttggcctagtcatagtaagc

SEQ ID NO: 961 - DNA, 54 bp, artificial sequence
  Synthetic
cgaccttaatgggcgaccagccgccagagcggaatcgccggatcaacgacaacc

SEQ ID NO: 962 - DNA, 54 bp, artificial sequence
  Synthetic
ccggagggtcagattactccctcggcttacccacgtggcctagtacctagagac

SEQ ID NO: 963 - DNA, 54 bp, artificial sequence
  Synthetic
cccggaagatcctccgccgtagtaacataagcacttagcccgagatcgagaagc

SEQ ID NO: 964 - DNA, 54 bp, artificial sequence
  Synthetic
ccggcacggtccgagaccccctcggctgacggagacgcccctagatcgagaagc

SEQ ID NO: 965 - DNA, 54 bp, artificial sequence
  Synthetic
cgggagatgaagccccaaccgagaagcctctctttccgcgaaccaagtctaagc

SEQ ID NO: 966 - DNA, 54 bp, artificial sequence
  Synthetic
ccggtgtcgggattactgcccggccgtgacggagtcgccgaaccaacgagaagc

SEQ ID NO: 967 - DNA, 54 bp, artificial sequence
  Synthetic
cgaaggaccgcttcacccggcgtccacatcggaatcgccgaatcaagtgtaatc

SEQ ID NO: 968 - DNA, 54 bp, artificial sequence
  Synthetic
ccggcacggtccgagaccccctcaagcctccctttcggcccgaattagagaacc

SEQ ID NO: 969 - DNA, 54 bp, artificial sequence
  Synthetic
ccggcagggtgcgagacattctcaaagtctgcacttaccctagtaatagcggag

SEQ ID NO: 970 - DNA, 54 bp, artificial sequence
  Synthetic
ccggcaagatcctagtccgatgtaacgtacccattcggcccgagaacgtgaagc

SEQ ID NO: 971 - DNA, 54 bp, artificial sequence
  Synthetic
ccggcatggaaccctcaaccgagaagcctctctttcgccccagtacctagaaac

SEQ ID NO: 972 - DNA, 54 bp, artificial sequence
  Synthetic
ccggcacgatccgagacattctcaaagtccgcacttggcctagtaatagcgagc

SEQ ID NO: 973 - DNA, 54 bp, artificial sequence
  Synthetic
ccgccgtcatgatcccgccccggtccggacggaatcgccgaatcaacgagaatc

SEQ ID NO: 974 - DNA, 54 bp, artificial sequence
  Synthetic
ccgggacggtccgagaccccctcaagcctccctttcggcccgaattagagaagc

SEQ ID NO: 975 - DNA, 54 bp, artificial sequence
  Synthetic
ccggagaggctgatgaccctctcggcttacgcactatgcctttcacgtattcgc

SEQ ID NO: 976 - DNA, 54 bp, artificial sequence
  Synthetic
ccgccgtcatgatcgacattctcaaagtctgcacttggcctagtaatagcgatc

SEQ ID NO: 977 - DNA, 54 bp, artificial sequence
  Synthetic
ccggcacggtccgagaccccctctcgggacggaatcgccgaatcaacgagaatc

SEQ ID NO: 978 - DNA, 54 bp, artificial sequence
  Synthetic
ccggcacggtccgagaccccctcggctctcccattcggcccggtaatagaaatc

SEQ ID NO: 979 - DNA, 54 bp, artificial sequence
  Synthetic
cccgcacggtccgagacattctcaaagtctccacttggcccaataatagcgtgc

SEQ ID NO: 980 - DNA, 54 bp, artificial sequence
  Synthetic
cccggaagatcctccgccgtagtaacataagcacttagcctagtaatagcgatc

SEQ ID NO: 981 - DNA, 54 bp, artificial sequence
  Synthetic
ccggcacggtccgagacattctcaaagtctgcacttggcctagtaatagtaagc

SEQ ID NO: 982 - DNA, 54 bp, artificial sequence
  Synthetic
cccgcccgtttccctcaacaggcaagccgccctttcggcccgaatttgagaatc

SEQ ID NO: 983 - DNA, 54 bp, artificial sequence
  Synthetic
ccggcacggctgatgacccccctcacaatctcacttgtcctaataatcgcaagc

SEQ ID NO: 984 - DNA, 54 bp, artificial sequence
  Synthetic
agcagtgcgatgacggcgaccgctacgctcccgttcggcccgagatcgagaatc

SEQ ID NO: 985 - DNA, 54 bp, artificial sequence
  Synthetic
ccggcatggtcttctcaacacgcaggcctccctttcggcccgaattagagaatc

SEQ ID NO: 986 - DNA, 54 bp, artificial sequence
  Synthetic
ccggcacggtccgagacattctcaaagtctccacttgtcctagtaatagcggag

SEQ ID NO: 987 - DNA, 54 bp, artificial sequence
  Synthetic
ccgccgtcatgatcccgccccgtaacttacccattcggccctagaattgtaagc

SEQ ID NO: 988 - DNA, 54 bp, artificial sequence
  Synthetic
cgccctcaatggccgaccagccgccgcgacccattcggcctagtaactagaagc

SEQ ID NO: 989 - DNA, 54 bp, artificial sequence
  Synthetic
ccggcgtcatgatcccgtcccgggcgttacccattcggccctagaacgagaagc

SEQ ID NO: 990 - DNA, 54 bp, artificial sequence
  Synthetic
cgacctcaatggccgagaagccgtcctgacccacttgtcctagtaacgagaacc

SEQ ID NO: 991 - DNA, 54 bp, artificial sequence
  Synthetic
ccgccgtcatgatcccgccccggtcgggaccctttcggcccgaattagagaatc

SEQ ID NO: 992 - DNA, 54 bp, artificial sequence
  Synthetic
ccggcgtgatgatcccgtcccgggcgttacccatacggccctagaacgagaagc

SEQ ID NO: 993 - DNA, 54 bp, artificial sequence
  Synthetic
ccaccccgtttaacccatgcttcctgactgccacttggcgtagtaacgagaaac

SEQ ID NO: 994 - DNA, 54 bp, artificial sequence
  Synthetic
ccggcacggtccgagacaacactcagcctcccacttggcctagtaatagcggag

SEQ ID NO: 995 - DNA, 54 bp, artificial sequence
  Synthetic
cgacctcaatggccgaccagccgccgtgacccattcgtcgctacatcgagaagc

SEQ ID NO: 996 - DNA, 54 bp, artificial sequence
  Synthetic
ccggcgtcatgatcccgtcccgggcgttccccatacggccctagaacgagaagc

SEQ ID NO: 997 - DNA, 54 bp, artificial sequence
  Synthetic
acggcacggtccgagacattctgcaagactgcagttggcctcgtaatcgaaatc

SEQ ID NO: 998 - DNA, 54 bp, artificial sequence
  Synthetic
cccgcagggtgggagacccacgtaagttacccattcggccctaatacgagaagc

SEQ ID NO: 999 - DNA, 54 bp, artificial sequence
  Synthetic
ccggccgggtccgagaaattgttaaactctgcacttggcctagtaatacgcagc

SEQ ID NO: 1000 - DNA, 54 bp, artificial sequence
  Synthetic
acgagcgactctactgacatccggtgtctcccgttcggcccgagatcgagaatc

SEQ ID NO: 1001 - DNA, 54 bp, artificial sequence
  Synthetic
acaggccgatgatcccgccccggtcgggacggaatcgccgaatcaagtgtgcgc

SEQ ID NO: 1002 - DNA, 54 bp, artificial sequence
  Synthetic
ccggcacggtccgagacattctcaaagtctgcacttggcctagcaattgtaagc

SEQ ID NO: 1003 - DNA, 54 bp, artificial sequence
  Synthetic
ccgggaagatcctgctccgaggtaacttacccattcggccctagaacgaggagc

SEQ ID NO: 1004 - DNA, 54 bp, artificial sequence
  Synthetic
ccggcacattccgagacattcactgcctattcatttcgcctaccaacgagaatc

SEQ ID NO: 1005 - DNA, 54 bp, artificial sequence
  Synthetic
ccgccgtcatgatcccgccccgtaacttacccattcggccctagaacgagaagc

SEQ ID NO: 1006 - DNA, 54 bp, artificial sequence
  Synthetic
cccggacgctgagaccgcccctcggcttacgcagacgccgaaccaagggtaagc

SEQ ID NO: 1007 - DNA, 54 bp, artificial sequence
  Synthetic
acaggacgatgcaactttcgtcatccagacggagacgccgaaccaatggtcagc

SEQ ID NO: 1008 - DNA, 54 bp, artificial sequence
  Synthetic
ccagcgcgctccgagacccccgctacttacccattcggccctagatcgagaacc

SEQ ID NO: 1009 - DNA, 54 bp, artificial sequence
  Synthetic
atggcacaacgaggtaacgaagtaacgttcccattcggcccgaattagagaagc

SEQ ID NO: 1010 - DNA, 54 bp, artificial sequence
  Synthetic
cccgggagctccgagacattcgcaacttacccattcggccctagaacgagaagc

SEQ ID NO: 1011 - DNA, 54 bp, artificial sequence
  Synthetic
ccgggaagatcctagtccgaggtaacttacccattcggcccgagatatagaaac

SEQ ID NO: 1012 - DNA, 54 bp, artificial sequence
  Synthetic
ccgatgtcatgatcttcccccggtagcgacccattcggccctagaacgagaagc

SEQ ID NO: 1013 - DNA, 54 bp, artificial sequence
  Synthetic
ccgccgtcatcatcccgccccggtcgggacggaatcgccgaatcaagtgtaatc

SEQ ID NO: 1014 - DNA, 54 bp, artificial sequence
  Synthetic
ccggcacggtgcgagacccccgtaacttacccattcggccctagaacgagaagc

SEQ ID NO: 1015 - DNA, 54 bp, artificial sequence
  Synthetic
cccggaagatcctccgcattctcaaagtctgcacttggcctagtaatagcggag

SEQ ID NO: 1016 - DNA, 54 bp, artificial sequence
  Synthetic
ccggcacggctcagggtactctcggcttacgcagacgccgaaccaagggtaagc

SEQ ID NO: 1017 - DNA, 54 bp, artificial sequence
  Synthetic
ccggcagggtgcgagacccccgggcgttacccatacggccctagaacgagaagc

SEQ ID NO: 1018 - DNA, 54 bp, artificial sequence
  Synthetic
ccggcacgatccgagacattcacaaactctgcacttggcctagtaatagcgagc

SEQ ID NO: 1019 - DNA, 54 bp, artificial sequence
  Synthetic
ccggcacggtccgagaccccctcaagcctccctttcggcccgaattaggagagc

SEQ ID NO: 1020 - DNA, 54 bp, artificial sequence
  Synthetic
ccggcacggtccgagacattctcaaagtctgcactcggcccgaattagagaatc

SEQ ID NO: 1021 - DNA, 54 bp, artificial sequence
  Synthetic
ccggcacgatccgagacattctcaaagtctccacttggactagtaatagcgagc

SEQ ID NO: 1022 - DNA, 54 bp, artificial sequence
  Synthetic
cccgggatctccgagacattcgctaagactgcagttggcctagtaacgagaatc

SEQ ID NO: 1023 - DNA, 54 bp, artificial sequence
  Synthetic
ccggcgtcatgttcccgtccccgccgttacccatacggccctggcactagaagc

SEQ ID NO: 1024 - DNA, 54 bp, artificial sequence
  Synthetic
ccggcgtcatgatcccgtcccgggcgttacccatacggccctagaagtatcagc

SEQ ID NO: 1025 - DNA, 54 bp, artificial sequence
  Synthetic
ccggcacggtccgatcaacaggcaagcctccctttcggcccgaattagagaagc

SEQ ID NO: 1026 - DNA, 54 bp, artificial sequence
  Synthetic
ccggcacagcaccccaaaccgaggagcctccctttcggcctaagaacgcgaagc

SEQ ID NO: 1027 - DNA, 54 bp, artificial sequence
  Synthetic
ccggcgtcatgatcccgtcacgggcgttacccatacggccctagaacgagaagc

SEQ ID NO: 1028 - DNA, 54 bp, artificial sequence
  Synthetic
ccgggaggctcgatgagccaccctgcctatacattcgacgaatcaagtagcggc

SEQ ID NO: 1029 - DNA, 54 bp, artificial sequence
  Synthetic
ccgggacgctccgagaccctctcaaagtctgcacttggcctagtcatagccagc

SEQ ID NO: 1030 - DNA, 54 bp, artificial sequence
  Synthetic
ccagcaaggtccactccatccgaagtttactcaaagtcccaatcaactgtaagc

SEQ ID NO: 1031 - DNA, 54 bp, artificial sequence
  Synthetic
ccggcagggtgcgagaccaccgtaactgacccattcggccctagaacgagtcgc

SEQ ID NO: 1032 - DNA, 54 bp, artificial sequence
  Synthetic
cgacctcaatggccgaccagccgccagagcggaatcgccgtgagttcgataatc

SEQ ID NO: 1033 - DNA, 54 bp, artificial sequence
  Synthetic
ccggagagctccgagacattcgcaacttacccattcggccatagaacgtgaagc

SEQ ID NO: 1034 - DNA, 54 bp, artificial sequence
  Synthetic
cccgcagaagaggattaagagggccgtgacccattcggccctagatcagcaagc

SEQ ID NO: 1035 - DNA, 54 bp, artificial sequence
  Synthetic
ggcgagctcttggtagtacccgctcccctcccgttcggcccgagatcgagaatc

SEQ ID NO: 1036 - DNA, 54 bp, artificial sequence
  Synthetic
acggcgacatccgagacattcgctaagactgcacttggcctagtaatagcggag

SEQ ID NO: 1037 - DNA, 54 bp, artificial sequence
  Synthetic
atgacactacgaggtaagtcccgtctactcccgttcggcccgagatcgagaatc

SEQ ID NO: 1038 - DNA, 54 bp, artificial sequence
  Synthetic
ccgccgtcatgatcccgctccggtcaggacggaatcgtcgaatgaacgtgaagc

SEQ ID NO: 1039 - DNA, 54 bp, artificial sequence
  Synthetic
ccggcacggccggcccagatgagaaacctctcaattgtcctagcaataggatgc

SEQ ID NO: 1040 - DNA, 54 bp, artificial sequence
  Synthetic
acgggccgatgatcccgccccggtcgggacggaatcgccgaatcaacgagaaac

SEQ ID NO: 1041 - DNA, 54 bp, artificial sequence
  Synthetic
cgacctcaatggccgaccagccgccagagcggaatcggcctagcaatagcaagc

SEQ ID NO: 1042 - DNA, 54 bp, artificial sequence
  Synthetic
cccgcccctataaggacatactgagttgacgcagagtcccaaccaattgtcagc

SEQ ID NO: 1043 - DNA, 54 bp, artificial sequence
  Synthetic
cctgcgtcatgatcccgccccgatcgggacggaatcgccgaatgaacgagaacc

SEQ ID NO: 1044 - DNA, 54 bp, artificial sequence
  Synthetic
acggcacggtccgagacattcgcaaagtctgcacttggactagtaatagcgcag

SEQ ID NO: 1045 - DNA, 54 bp, artificial sequence
  Synthetic
ccggcaaggaccgtgaccccccccgctcgcccatttggcgtggtaataaaaaac

SEQ ID NO: 1046 - DNA, 54 bp, artificial sequence
  Synthetic
ccggcacgatccgagacaagccgccagagcggaatcgccgaatcaacgagaacc

SEQ ID NO: 1047 - DNA, 54 bp, artificial sequence
  Synthetic
aacgccccgggcatcaagtattgtagttacccactcggcccgagaacgagaagc

SEQ ID NO: 1048 - DNA, 54 bp, artificial sequence
  Synthetic
ccgccgtcatgatcccgccccggtcgggacggaatcgccgaaagatcgagaatc

SEQ ID NO: 1049 - DNA, 54 bp, artificial sequence
  Synthetic
cgcggaagatcctcgtccgaagtaacgttcccattcggcccgaattagagaatc

SEQ ID NO: 1050 - DNA, 54 bp, artificial sequence
  Synthetic
acgaatcaatggccgaccagccgccagagcggaatcgccgaatcaacgagaacc

SEQ ID NO: 1051 - DNA, 54 bp, artificial sequence
  Synthetic
ccgccgtcatgatcccgccccggtcgggacggaatcggcctaagatcgagaatc

SEQ ID NO: 1052 - DNA, 54 bp, artificial sequence
  Synthetic
ccggcccggtccgagacattgtcaaagtctgtacttggcctggcaacgacaaac

SEQ ID NO: 1053 - DNA, 54 bp, artificial sequence
  Synthetic
ccggcacggtcctcacccggcgtccacatcccattcggccctagaacgagaagc

SEQ ID NO: 1054 - DNA, 6 bp, artificial sequence
  Synthetic
cacttg

SEQ ID NO: 1055 - DNA, 11 bp, artificial sequence
  Synthetic
cctagtaatag

SEQ ID NO: 1056 - DNA, 4 bp, artificial sequence
  Synthetic
ccgg

SEQ ID NO: 1057 - DNA, 4 bp, artificial sequence
  Synthetic
tttt

SEQ ID NO: 1058 - DNA, 7 bp, artificial sequence
  Synthetic
  misc_feature (1)..(2): n is a, c, g, or t
nnagaaw

SEQ ID NO: 1059 - DNA, 8 bp, artificial sequence
  Synthetic
  misc_feature (1)..(4): n is a, c, g, or t
nnnngatt

SEQ ID NO: 1060 - DNA, 6 bp, artificial sequence
  Synthetic
  misc_feature (1)..(2): n is a, c, g, or t
nnagaa

SEQ ID NO: 1061 - DNA, 6 bp, artificial sequence
  Synthetic
  misc_feature (1)..(1): n is a, c, g, or t
naaaac

SEQ ID NO: 1062 - DNA, 45 bp, artificial sequence
  Synthetic
catacgatgttccagattacgcttgaattcgtgggaattggctcc

SEQ ID NO: 1063 - DNA, 20 bp, artificial sequence
  Synthetic
agacaaaggcttggccatgg

SEQ ID NO: 1064 - DNA, 45 bp, artificial sequence
  Synthetic
ccggccaggcaaaaaagaaaaagtgaattcgtgggaattggctcc

SEQ ID NO: 1065 - DNA, 39 bp, artificial sequence
  Synthetic
acttttgtcttatacttggatcaccggtgccaccatggc

SEQ ID NO: 1066 - DNA, 49 bp, artificial sequence
  Synthetic
ccggagccaattcccacgaattcagcgtaatctggaacatcgtatgggt

SEQ ID NO: 1067 - DNA, 86 bp, artificial sequence
  Synthetic
cttttgtcttatacttggatcaccggtgccaccatggccccaaagaagaagcggaaggtcggcagcacacagttcgagggctttac

SEQ ID NO: 1068 - DNA, 46 bp, artificial sequence
  Synthetic
ccggagccaattcccacgaattcagcgtaatctggaacatcgtatg

SEQ ID NO: 1069 - DNA, 38 bp, artificial sequence
  Synthetic
acttttgtcttatacttggatcaccggtgccaccatgg

SEQ ID NO: 1070 - DNA, 45 bp, artificial sequence
  Synthetic
cggagccaattcccacgaattcctttttcttttttgcctggccgg

SEQ ID NO: 1071 - DNA, 54 bp, artificial sequence
  Synthetic
tttggactgcacgacagtcgacgatggtttcgacacgactcgcgcatacgatgg

SEQ ID NO: 1072 - DNA, 54 bp, artificial sequence
  Synthetic
tttggactacagtcgctacgacgatggtttcgcgagcgctatgagcgactatgg

SEQ ID NO: 1073 - DNA, 54 bp, artificial sequence
  Synthetic
tttggatacgatacgcgcacgctatggtttcgagagcgcgctcgtcgactatgg

SEQ ID NO: 1074 - DNA, 53 bp, artificial sequence
  Synthetic
tcgtcggcagcgtcagatgtgtataagagacagtatgtgtgggagggctaaac

SEQ ID NO: 1075 - DNA, 57 bp, artificial sequence
  Synthetic
gtctcgtgggctcggagatgtgtataagagacagtgtaatccagaggttgattgtcg

SEQ ID NO: 1076 - DNA, 20 bp, artificial sequence
  Synthetic
gactatcatatgcttaccgt

SEQ ID NO: 1077 - DNA, 20 bp, artificial sequence
  Synthetic
tgcttacgatggagccagag

SEQ ID NO: 1078 - DNA, 23 bp, artificial sequence
  Synthetic
cttacgatggagccagagaggat

SEQ ID NO: 1079 - DNA, 20 bp, artificial sequence
  Synthetic
tagagctactgcaattattc

SEQ ID NO: 1080 - DNA, 23 bp, artificial sequence
  Synthetic
gcctgaataattgcagtagctct

SEQ ID NO: 1081 - DNA, 20 bp, artificial sequence
  Synthetic
tttcccttcagctaaaataa

SEQ ID NO: 1082 - DNA, 23 bp, artificial sequence
  Synthetic
tttcccttcagctaaaataaagg

SEQ ID NO: 1083 - DNA, 43 bp, artificial sequence
  Synthetic
ccatctcatccctgcgtgtctccgtgttcagtctccgtgaacg

SEQ ID NO: 1084 - DNA, 44 bp, artificial sequence
  Synthetic
cctctctatgggcagtcggtgatgaagtcactctggggaacacg

SEQ ID NO: 1085 - DNA, 43 bp, artificial sequence
  Synthetic
ccatctcatccctgcgtgtctccggtgacacacccccatttcc

SEQ ID NO: 1086 - DNA, 44 bp, artificial sequence
  Synthetic
cctctctatgggcagtcggtgatgactgagaaccgggcaggtca

SEQ ID NO: 1087 - DNA, 43 bp, artificial sequence
  Synthetic
ccatctcatccctgcgtgtctcctctcttctgggctccctaca

SEQ ID NO: 1088 - DNA, 44 bp, artificial sequence
  Synthetic
cctctctatgggcagtcggtgatgagcagtgcgtcatcccaaga

SEQ ID NO: 1089 - DNA, 22 bp, artificial sequence
  Synthetic
cgtaatttctactcttgtagat

SEQ ID NO: 1090 - DNA, 23 bp, artificial sequence
  Synthetic
atccagtttggttaatttaatta

SEQ ID NO: 1091 - DNA, 67 bp, artificial sequence
  Synthetic
atcttgtggaaaggacgaaacaccgtaatttctactcttgtagatggagacggttgtaaatgagcac

SEQ ID NO: 1092 - DNA, 43 bp, artificial sequence
  Synthetic
tacgccttaattaaaaaagagacgtacaaaaaagagcaagaag

SEQ ID NO: 1093 - DNA, 43 bp, artificial sequence
  Synthetic
gtctcttttttaattaaggcgtaactagatcttgagacaaatg

SEQ ID NO: 1094 - DNA, 20 bp, artificial sequence
  Synthetic
ttatccatctttgcacccgg

SEQ ID NO: 1095 - DNA, 45 bp, artificial sequence
  Synthetic
ccatctcatccctgcgtgtctcccgtaatttctactcttgtagat

SEQ ID NO: 1096 - DNA, 47 bp, artificial sequence
  Synthetic
cctctctatgggcagtcggtgatgatccagtttggttaatttaatta

SEQ ID NO: 1097 - DNA, 26 bp, artificial sequence
  Synthetic
tcgtcggcagcgtcagatgtgtataa

SEQ ID NO: 1098 - DNA, 55 bp, artificial sequence
  Synthetic
gtctcgtgggctcggagatgtgtataagagacagtttctcctctcggagatttgc

SEQ ID NO: 1099 - DNA, 21 bp, artificial sequence
  Synthetic
ggtcatgcccggttcaccgaa

SEQ ID NO: 1100 - DNA, 21 bp, artificial sequence
  Synthetic
ctagctctagagaagtcctac

SEQ ID NO: 1101 - DNA, 16 bp, artificial sequence
  Synthetic
ggtcatgagcaccgaa

SEQ ID NO: 1102 - DNA, 13 bp, artificial sequence
  Synthetic
ctagtaggactac

SEQ ID NO: 1103 - DNA, 27 bp, artificial sequence
  Synthetic
gagtaacagacatggaccatcaggaaa

SEQ ID NO: 1104 - DNA, 27 bp, artificial sequence
  Synthetic
tttggcctgaataattgcagtagctct

SEQ ID NO: 1105 - DNA, 27 bp, artificial sequence
  Synthetic
atcctctctggctccatcgtaagcaaa

SEQ ID NO: 1106 - DNA, 11 bp, artificial sequence
  Synthetic
tttacctgaaa